(19) United States
(12) Patent Application Publication
(10) Pub. No.: US 2021/0380655 A1
(43) Pub. Date: Dec. 9, 2021

(54) PRODUCTION OF SOLUBLE RELAXIN AND RELAXIN ANALOGS

(71) Applicant: TRUSTEES OF BOSTON UNIVERSITY, Boston, MA (US)

(72) Inventors: Mark W. GRINSTAFF, Brookline, MA (US); Jack KIRSCH, Boston, MA (US); William BLESSING, Boston, MA (US)

(73) Assignee: TRUSTEES OF BOSTON UNIVERSITY, Boston, MA (US)

(21) Appl. No.: 17/335,357

(22) Filed: Jun. 1, 2021

Related U.S. Application Data
(60) Provisional application No. 63/033,318, filed on Jun. 2, 2020.

(51) Int. Cl. C07K 14/64; C12P 21/02

(52) U.S. Cl. CPC C07K 14/64; C07K 2319/50; C07K 2319/20; C12P 21/02

(57) ABSTRACT

Provided herein is a method to produce relaxin or relaxin analogues or variants and their use in methods of treatment, e.g., in the treatment of a stiffened fibrotic joint.

### CROSS-REFERENCE TO RELATED APPLICATIONS

This application claims benefit under 35 U.S.C. § 119(e) of U.S. Provisional Application No. 63/033,318 filed Jun. 2, 2020, the contents of which are incorporated herein by reference in its entirety.

### SEQUENCE LISTING

The instant application contains a Sequence Listing which has been submitted electronically in ASCII format and is hereby incorporated by reference in its entirety. Said ASCII copy, created on Jul. 26, 2021, is named 701586-097790USPT_SL.txt and is 38,173 bytes in size.

### TECHNICAL FIELD

The technology described herein relates to methods for production of soluble relaxin and relaxin analogs, compositions comprising same and their use in treating fibrotic diseases.

### BACKGROUND

Joint stiffness is a significant public health issue with current treatment options providing varied and limited outcomes. Joint stiffness can affect any joint in the body, such as a shoulder joint, an elbow joint, a wrist joint, a finger joint, a hip joint, a knee joint and an ankle joint. A shoulder joint is often affected by joint stiffness, which is also termed a shoulder contracture, and is also known as “frozen shoulder”.

Shoulder contracture affects approximately 2% of the U.S. population, or approximately six million individuals. While women are more often affected than men, there is no known genetic or racial predilection (Robinson C. M. et al, J. Bone Joint Surg. Br. 2012, 94(1):1-9; Ewald A., Am. Fam. Physician 2011, 83(4):417-22). Shoulder contracture recovery is arduous and protracted with a significant number of patients never regaining full joint function. The condition affects both quality of life and productivity. Its predominant feature is painful, gradual loss of both active and passive glenohumeral motion resulting from progressive fibrosis of the glenohumeral joint capsule. The contracted capsule causes pain, especially when it is stretched suddenly, and produces a mechanical restraint to motion. The disease course of primary (idiopathic) shoulder contracture begins with the slow onset (over 2 to 9 months) of pain and stiffness that progressively restricts both passive and active range of motion (ROM) in the glenohumeral joint (Sharma S., Annals of the Royal College of Surgeons of England 2011 93(5):343-4; discussion 5-6). The pain may sharpen at night, leaving patients unable to sleep on the affected side. Subsequently, the pain generally abates over a period of 4 to 12 months, but stiffness severely restricts ROM, particularly in the external rotational plane. There is a slow improvement in ROM over a period of 2 to 4 years. Secondary shoulder contracture has a similar presentation and progression but results from a known intrinsic or extrinsic cause (Sheridan M. A. and Hannafin J. A., Orthop. Clin. North Am. 2006, 37(4):531-9). Secondary shoulder contracture following trauma or surgery has a 100% incidence to varying degrees after these events and requires prolonged physical therapy, with original motion not always restored.

Shoulder contracture pathology is a thickened glenohumeral joint capsule with adhesions obliterating the axillary fold. The fibrotic capsule adheres to itself and the anatomic neck of the humerus, intraarticular volume is diminished, and synovial fluid in the joint is significantly decreased (Hand G. C. et al., J. Bone Joint Surg. Br. 2007, 89(7):928-32). Biopsy of the capsule shows a chronic inflammatory infiltrate, an absence of synovial lining, and subsynovial fibrosis (Ozaki J. et al., J. Bone Joint Surg. Am. 1989, 71(10):1511-5; Wiley A. M., Arthroscopy 1991, 7(2):138-43; Rodeo S. A. et al., J. Orthop. Res. 1997, 15(3):427-36). Patient biopsy samples confirm the presence of T-cells, B-cells, synovial cells, fibroblasts and transforming myofibroblasts, along with type-I and type-III collagen (Rodeo S. A. et al., J. Orthop. Res. 1997, 15(3):427-36; Bunker T. D. et al., J. Bone Joint Surg. Br. 2000, 82(5):768-73). Gene and protein expression assays have found products related to fibrosis, inflammation, and chondrogenesis (Hagiwara Y. et al., Osteoarthritis Cartilage 2012, 20(3):241-9), including increased COL1A1 and COL1A3, interleukin-6, platelet-derived growth factor (PDGF), fibroblast growth factors (FGF) and inhibitors of the matrix metalloproteinases (TIMPs), as well as decreased activity of matrix metalloproteinases (MMPs). These data indicate that inflammatory changes initiate the recruitment of fibroblasts and immune cells, precipitating the fibrotic process and inappropriate deposition of collagen. Alternatively, fibrotic changes may occur first, followed by inflammation. In this case, fibrosis may result from an underlying disease process, in which cell signaling pathways governing collagen remodeling may be defective (Bunker T. D. et al., J. Bone Joint Surg. Br. 2000, 82(5):768-73). For example, patients treated with marimastat, a synthetic TIMP, developed shoulder contractures, and when the marimastat was stopped, the disease regressed (Hutchinson J. W. et al., J. Bone Joint Surg. Br. 1998, 80(5):9078).

Shoulder contracture is considered a self-limiting disease, but recovery is protracted and arduous, with a significant number of patients never regaining full ROM. The reported outcomes of conservative therapy (i.e., physical therapy) vary considerably and are highly dependent on how they are measured (Neviaser A. S. and Neviaser R. J., J. Am. Acad. Orthop. Surg. 2011, 19(9):536-42). Results tend to be more favorable with subjective outcome measures than with objective outcome measures. In one study, 90% of patients treated with minimal therapy reported satisfaction with their shoulder function (Griggs S. M. et al., J. Bone Joint Surg. Am. 2000, 82-A(10):1398-407). However, another that used objective outcomes reported residual pain in 50% of patients and motion deficit in 60% (Shaffer B. et al., J. Bone Joint Surg. Am. 1992; 74(5):738-46). Mild to moderate symptoms can persist after 4.4 years following symptom onset of shoulder contracture. For those experiencing severe disease, such functional impairment interferes with daily activities and work-related responsibilities (Hand C. et al., Journal of Shoulder and Elbow Surgery 2008, 17(2):231-6). When patients do not respond to conservative management, other treatment options are available. Operative intervention in the form of manipulation under anesthesia may restore motion and decrease pain, but it has been associated with complications such as fracture, tendon rupture, and neurologic injury (Castellarin G. et al., Archives of Physical Medicine and Rehabilitation 2004, 85(8):1236-40; Hsu S. Y. and Chan K. M., International Orthopaedics, 1991, 15(2):79-83; Parker R. D. et al., Orthopedics, 1989, 12(7):989-90). There are reports that manipulation or capsular release do not offer reliable and consistent results (Shaffer B. et al., J. Bone Joint Surg. Am. 1992, 74(5):738-46; Ryans I. et al., Rheumatology 2005, 44(4):529-35). Accordingly, a more effective and consistent therapy for joint stiffness is needed. See also the Dissertation defense of William Blessing (Boston University) dated Aug. 26, 2019; the contents of which is incorporated herein by reference in its entirety.

Human Relaxin-2 is a naturally occurring peptide hormone in the insulin superfamily. Among many of the potential therapeutic indications for relaxin-2 is tissue fibrosis. It is an approximately 6.3 kDa protein comprised of two separate chains. The active form of the peptide hormone is made up of an A chain, with one intrachain disulfide bond, and a B chain, bound to the A chain through two interchain disulfide bonds. Each chain contains a-helical secondary structures, which are then linked via the previously mentioned disulfide bonds. In the human body relaxin-2 is produced as a pre-prohormone requiring post-translational processing by prohormone convertase to gain biological activity. Prohormone convertase cleaves the pre-prohormone, excises a peptide linking the A and B chain, and allows for refolding of the two chains.

Relaxin-2 has been studied as a therapeutic for over forty years, and during that time a variety of different production schema have been carried out. Relaxin-2 has been harvested from ex vivo tissue, expressed recombinantly, and chemically synthesized. Relaxin-2 has been isolated from the reproductive tissues of several vertebrate animals including but not limited to rats (Sherwood, O. D., Endocrinology 1979, 104(4):886-892.). Relaxin-2 has also been expressed and purified recombinantly as is described in U.S. Pat. No. 5,759,807 which describes a method to purify an insoluble relaxin requiring extensive chromatography and time-demanding denaturing and re-folding procedures.

There remains a need in the art for methods and compositions, which bypasses the need for primary tissues, that produces biologically active relaxin-2 in a simplified manner. Further, the ability to post-translationally functionalize relaxin-2 in a facile manner is desired for expanding clinical applications. The present invention addresses some of these needs.

### SUMMARY

The present invention provides methods to produce relaxin or relaxin analogs for use in the treatment of a stiffened fibrotic joint. The relaxin compositions include relaxin-2, a relaxin-2 variant, relaxin-2 chemically conjugated to a targeting agent, including a single-domain camelid antibody fragment, a peptide sequence, polynucleotide, synthetic polymer, or a small molecule. The invention describes the production and purification of an insulin family peptide, relaxin-2 or its analogs using recombinant methodology. The relaxin-2 or analog produced using this method is then administered intravenously, intramuscularly, subcutaneously, intradermally, intranasally, orally, transcutaneously, mucosally, or intraarticularly with or without a carrier or depot for the treatment of a stiffened fibrotic Joint.

Without wishing to be bound by a theory, exemplary methods described herein produce relaxin-2 or its analogs using a strain of E. coli and affords milligrams of soluble protein, sidestepping a previously published requirement to purify relaxin-2 from denaturing and refolding inclusion bodies.

Accordingly, in one aspect, the invention provides a method for recombinant production where the protein, e.g., a fusion protein comprising relaxin is secreted from cells.

In some embodiments, the relaxin-2 or its analogs is purified using one or more affinity tags that are subsequently cleaved and removed from relaxin prior to use. The affinity tags enable purification of the relaxin via the use of affinity resins and/or column chromatography.

In some aspects, the relaxin-2 or its analog has been recombinantly produced in a bacterial, mammalian or yeast host cell.

In other aspects the relaxin-2 or its analogs have been prepared via combining a partially chemically synthesized entity and a recombinantly produced entity.

In some aspects, the relaxin-2 or its analog is greater than 50, 55, 60, 65, 70, 75, 80, 85, 90, or 95% pure by chromatography.

In some aspects, the relaxin-2 or its analog is dissolved at a known concentration in PBS or saline and then used in the treatment of a stiffened fibrotic joint. In another aspect the relaxin-2 or its analog is freeze-dried and stored at 4-37° C. prior to reconstitution in PBS or saline and used in the treatment of a stiffened fibrotic joint. Relaxin is stable in solution over a wide range from 4 to 40+° C.

In some embodiments, the stiffened joint is selected from the group consisting of a shoulder joint, an elbow joint, a wrist joint, a finger joint, a hip joint, a knee joint, or an ankle joint. In one embodiment, the stiffened joint is a shoulder joint.

In some embodiments, the stiffened joint results from an injury, a medical procedure, an inflammation of the joint, prolonged immobility, a disease, or idiopathically.

In some aspects, the relaxin-2 or its analog is administered during a medical procedure, e.g., during surgery. In one embodiment, the relaxin 2 or its analog is loaded into a depot as a pellet form and is administered through a cannula or an incision. In another embodiment, the relaxin-2 or its analog loaded depot is administered during an outpatient fluoroscopic or ultrasound guided procedure.

In some embodiments, the relaxin-2 or its analog loaded depot is administered transcutaneously, e.g., using iontophoresis or electrophoresis. In one aspect, the relaxin 2 or its analog loaded depot is administered as a gel, a cream, an ointment, a lotion, a drop, a suppository, a spray, a liquid or a powder composition. In one aspect, the relaxin 2 or its analog loaded depot is administered intraarticularly.

In one aspect, provided herein is a method for producing recombinant relaxin or a variant or analogue thereof. Generally, the method comprises recombinantly expressing a fusion protein in a host cell. In some embodiments, the fusion protein comprises: a first affinity tag; a solubility domain; a protease cleavable domain; a relaxin domain; a self-cleaving domain; and a second affinity tag, optionally, wherein the first and second affinity tags are different. The expressed fusion protein is released from the host cell. The protease cleavable domain of the released is cleaved to release the solubility domain from the fusion protein. The self-cleaving domain is cleaved to release the relaxin domain. The released relaxin domain can optionally be cleaved to produce the A and B chains. The cleaved relaxin domain is subjected to oxidation-reduction conditions to produce soluble relaxin.

In another aspect, the invention provides relaxin produced by a method described herein.

In yet another aspect, the invention provides composition and kits comprising relaxin or a variant or produced by a method described herein.

In still another aspect, the invention provides a fusion protein comprising a relaxin domain and at least one of a solubility domain or a self-cleaving domain. Generally, the solubility domain is linked to the relaxin domain via a protease cleavable linker. In some embodiments, the fusion protein comprises a relaxin domain and a self-cleaving domain, and wherein the fusion protein optionally further comprises a solubility domain linked to the relaxin domain and/or at least one affinity tag, and wherein the solubility domain is linked to the relaxin domain via protease cleavable linker. In some embodiments, the fusion protein comprises a relaxin domain and a solubility domain linked to the relaxin domain, and wherein the fusion protein optionally further comprises a self-cleaving domain and/or at least one affinity tag, and wherein the solubility domain is linked to the relaxin domain via protease cleavable linker.

Compositions, kits and/or cells comprising a fusion protein described herein are also provided.

In yet still another aspect, the invention provides a polynucleotide encoding a fusion protein described herein. Compositions, kits and/or cells comprising a polynucleotide described herein are also provided.

### BRIEF DESCRIPTION OF THE DRAWINGS

FIG. 1 is a schematic representation of a fusion protein according to an exemplary embodiment. FIG. 1 discloses “6× Histidine” as SEQ ID NO: 51.

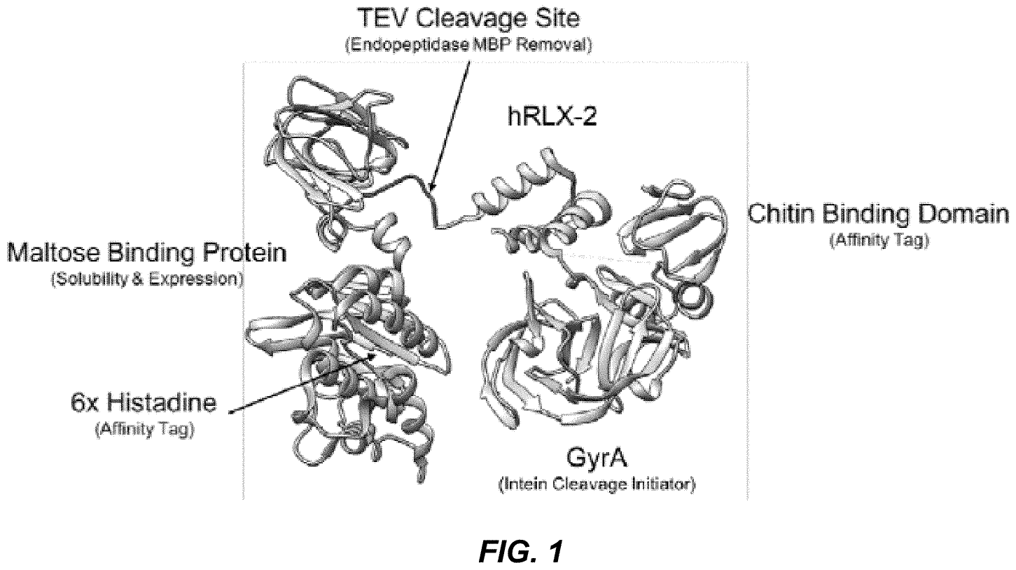

FIG. 2 shows purification of full relaxin protein from E. coli. 12% SDS PAGE showing the centrifugal fractionation of the full fusion protein. Lanes: 1) Protein Plus kaleidoscope ladder 2) whole cell lysate 3) insoluble fraction 4) soluble fraction 5) cobalt resin flow-through 6-8 cobalt resin wash 1-3.

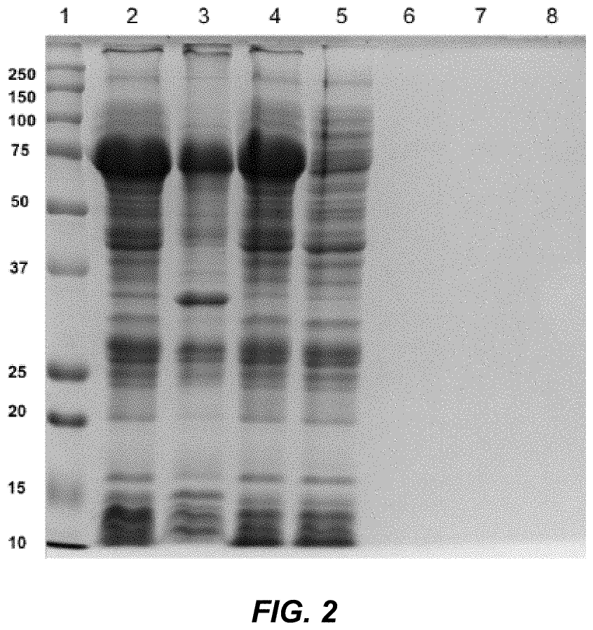

FIG. 3 shows un-pooled cobalt resin elution fractions from the relaxin production. 12% SDS PAGE. Lanes: 1) Protein plus kaleidoscope ladder 2-15) elution fraction, every other (7.5 ml fraction) Pooled fractions: [2-10, 21-25] show relaxin is prepared and clean.

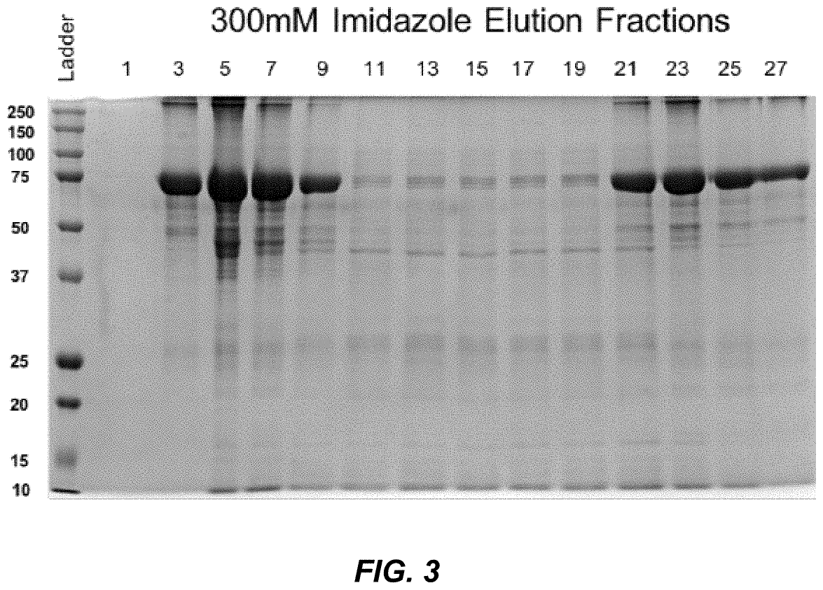

FIG. 4 shows removal of the solubility and expression domain. 16% SDS PAGE of TEV digest of the pooled cobalt elution fractions. Lanes: 1) Low range unstained protein ladder 2) TEV protease 3-6) 4, 24, 48, 72 hr TEV Digest 7) Post-chitin resin binding flow-through 8-10) chitin resin wash 1-3 11) MESNA wash 12) hRLX-2 control.

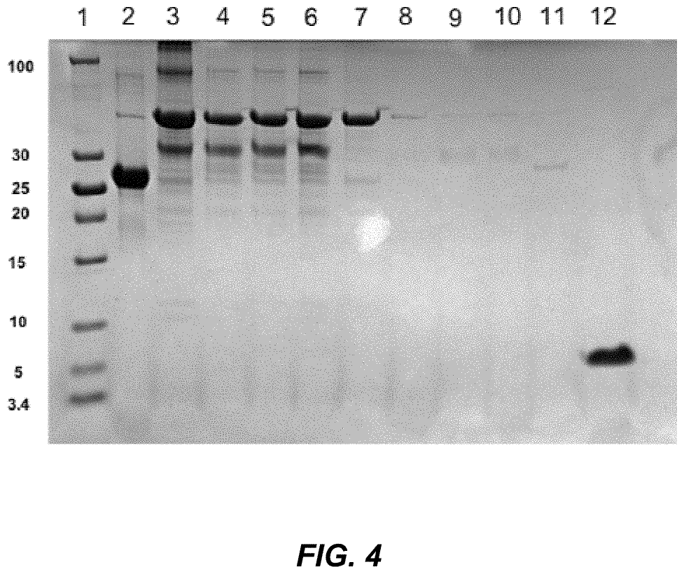

FIG. 5 shows digestion and refolding of rRLX-2. 16% SDS PAGE of recombinant RLX-2 after refolding under redox conditions (10 mM/2 mM Ox/Red glutathione) Lanes: 1) low range unstained protein ladder 2) Intein cleavage product—linearized relaxin 3) digested and refolded relaxin 4) digested and refolded relaxin+5% βME.

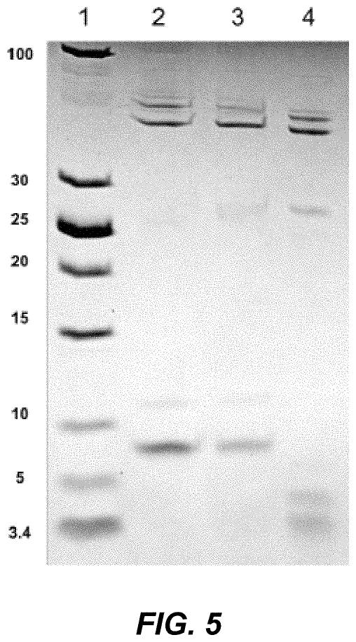

FIG. 6 shows confirmation of ELISA sensitivity to recombinantly produced refolded relaxin as determined by ELISA utilizing antibody matured against biologically active hRLX-2. Traditional sandwich ELISA.

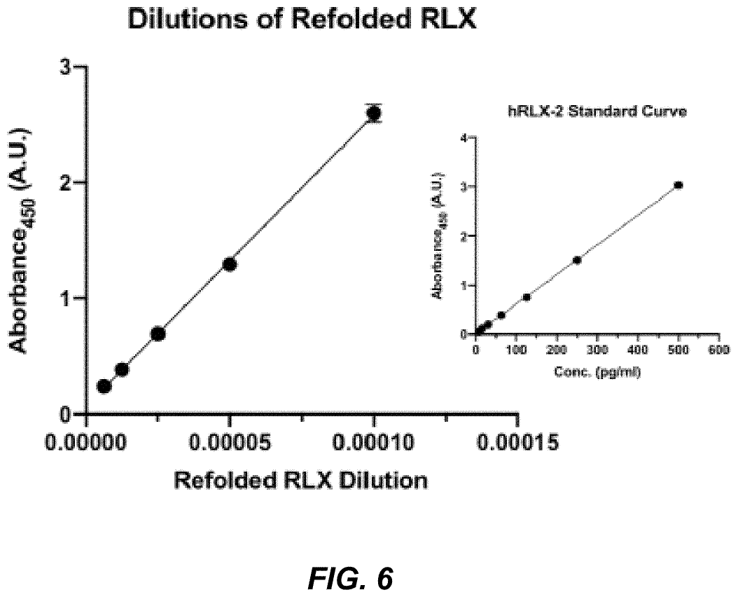

FIG. 7 shows in vitro activity of recombinantly produced Refolded-Relaxin. The in vitro activity of the recombinantly produced relaxin compared to hRLX-2 against collagen I production by human fibroblast like synoviocytes after 5 days of treatment.

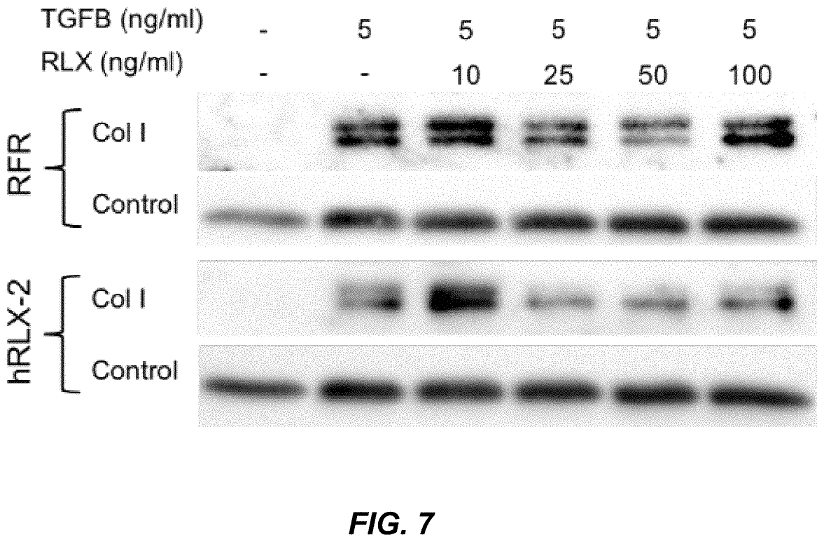

FIG. 8 shows in vitro activity of recombinantly produced Refolded-Relaxin. The in vitro activity of the recombinantly produced relaxin compared to hRLX-2 against collagen III production by human fibroblast like synoviocytes after 5 days of treatment.

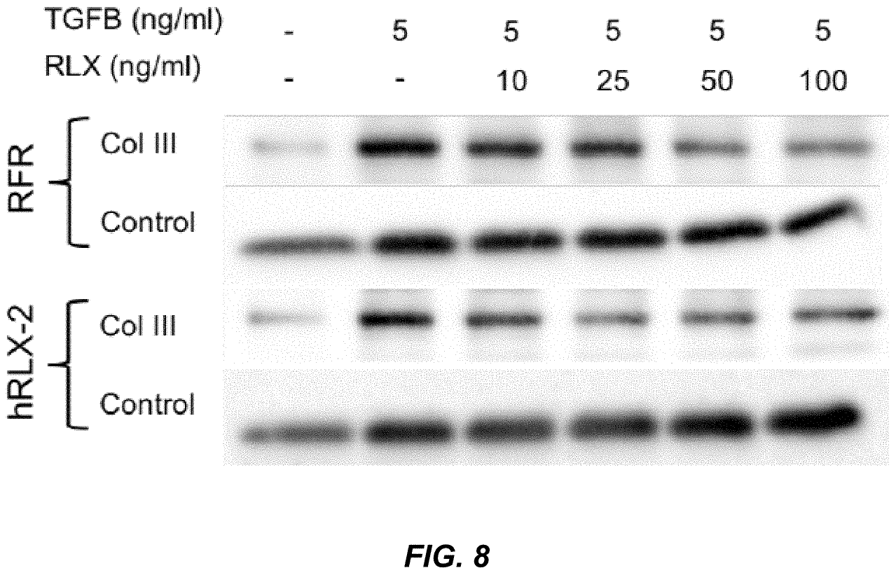

FIG. 9 shows in vitro activity of recombinantly produced Refolded-Relaxin. The in vitro activity of the recombinantly produced relaxin compared to hRLX-2. Total AKT cellular content human fibroblast like synoviocytes after 5 days of treatment.

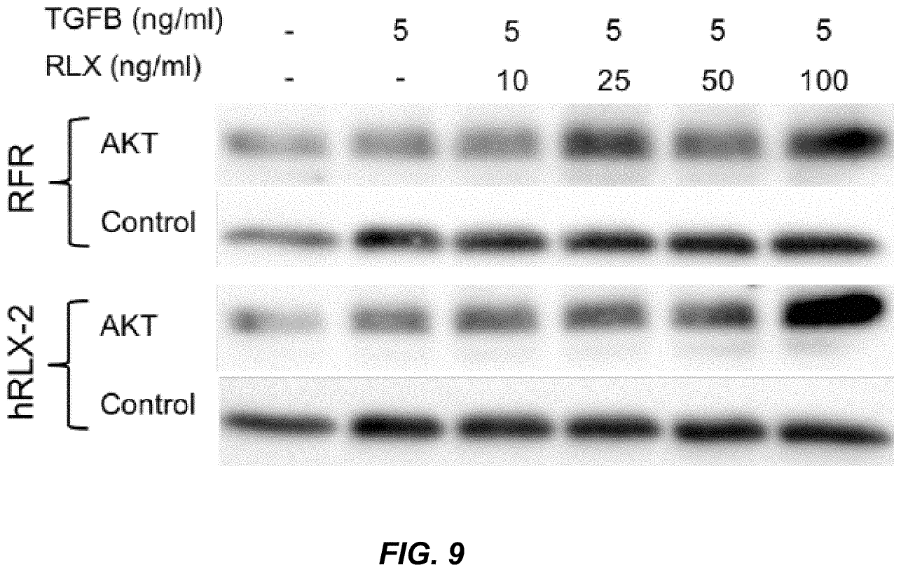

FIG. 10 shows in vitro activity of recombinantly produced Refolded-Relaxin. The in vitro activity of the recombinantly produced relaxin compared to hRLX-2. Total pERK cellular content human fibroblast like synoviocytes after 5 days of treatment.

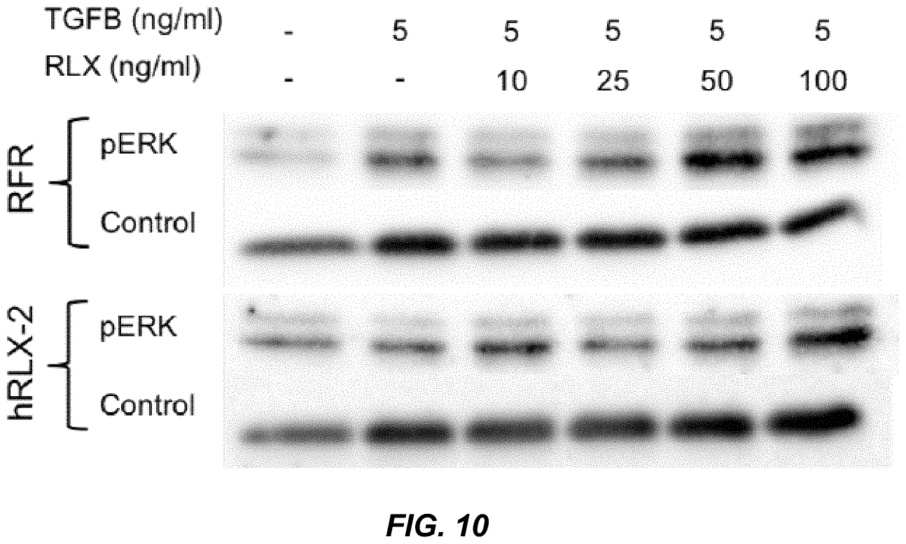

### DETAILED DESCRIPTION

The present invention provides methods for the production of recombinantly expressed, soluble relaxin or an analog or variant thereof. This method allows for the post-translational modification of the relaxin so chemical conjugation can be made between the relaxin and a targeting agent, including without limitation a single-domain camelid antibody fragment, a peptide sequence, polynucleotide, synthetic polymer, or a small molecule. The recombinant expression of relaxin or a variant thereof can be accomplished in eukaryotic or prokaryotic expression cell lines. Eukaryotic cells can include, but are not limited to mammalian, plant, insect, or yeast expression host. Prokaryotic cells can include Escherichia coli or bacteria of the Bacillus genus. In a specific embodiment of this method, the expression host is a E. coli cell line that is compatible with T7 polymerase.

The recombinant expression of relaxin or a variant thereof is expressed intracellularly through the use of a plasmid containing the coding sequence for the relaxin. The plasmid is transformed into the expression host using any variety of techniques familiar to a person skilled in the art including but not limited to heat shock, electroporation, calcium ion shock, viral infection, or microinjection. The plasmid can confer antibiotic or nutrient selection to the expression host, which allows for selective growth of only expression host cells containing the proper construct. The plasmid can contain an inducible promoter region or internal ribosomal entry site upstream of the gene of interest. The plasmid can internally contain an origin of replication.

Antibiotic agents allowing for the selective growth of expression hosts for recombinant expression can include, but are not limited to the bacterial antibiotic selection agents Actinomycin D, Ampicillin, Carbenicillin, Chloramphenicol, Gentamycin, Kanamycin, Puromycin, Streptomycin, Tetracycline or any combination thereof. Eukaryotic antibiotic selection agents can include, but are not limited to Blasticidin, Geneticin, Hygromycin, Puromycin, Zeocin, or any combination thereof. Eukaryotic selection markers can include HIS3, URA3, LYS2, LEU2, TRP1, MET15, ura4+, leu1+, ade6+.

Expression host cells are grown in a manner familiar to person skilled in the art, dependent on the nature of the expression host. In a specific embodiment involving the expression of the relaxin from a prokaryotic expression host, the expression of the relaxin is induced through the addition of Isopropyl β-D-1-thiogalactopyranoside (forward: IPTG) during the log phase of E. coli growth. Induction concentration can range from 0.01 mM to 10 mM IPTG. Bacterial cell density at the point of induction yields an optical density (600 nm) of 0.4-0.8. Induction of the relaxin can be carried out at between 4° C. and 37° C. with preferred temperatures of 4° C., 16° C., 20° C., 25° C., 30° C., and 37° C. Induction of the relaxin can be carried out for 1-24 hours.

The crude recombinant protein is separated from the expression host. In one embodiment, this is accomplished by harvesting the cell culture supernatant, which contains an excreted relaxin. In another embodiment, an osmotic shock procedure familiar to a person skilled in the art is carried out to isolate the relaxin expressed in the periplasmic space of a bacterial expression host. In another embodiment, the expression host is lysed in a manner familiar to a person skilled in the art. The method of lysis can include mechanical, chemical, thermal, enzymatic, or a combination thereof. In some specific embodiments of this method, the soluble, crude recombinant protein is separated from cellular material via centrifugation.

The present methods incorporate protein structure and/or size modification through the use of site specific proteases. The protease cleavage sequence that can be used include but are not limited to, Glu-Xaa-Xaa-Tyr-Xaa-Gln-Ser (SEQ ID NO: 58), Glu-Xaa-Xaa-Tyr-Phe-Xaa-Gly (SEQ ID NO: 59), Asp-Asp-Asp-Asp-Lys (SEQ ID NO: 3), or Arg-Gly-Yaa-Zaa (SEQ ID NO: 4), where Xaa denotes any amino acid, Yaa denotes any negatively charged amino acid. Zaa describes a cationic or neutral amino acid.

The methods of the invention utilize single or multiple rounds of successive affinity chromatography for the purification, clarification, or modification of the relaxin from the crude, semi-crude, or purified recombinant protein. These purification steps include, but are not limited to the use of anion exchange chromatography, cation exchange chromatography, boronate affinity chromatography, lectin affinity chromatography, immunoaffinity chromatography, immobilized metal affinity chromatography, protein affinity chromatography, and size exclusion chromatography.

In a specific embodiment, crude, soluble cell lysate is incubated with a crosslinked agarose resin containing immobilized metal ions. In this same embodiment the relaxin is eluted from the resin and then processed with a protease to separate a solubility domain from the domain containing relaxin. The fraction containing the solubility domain is discarded. The fraction containing the relaxin is then incubated with chitin resin, or similar. On-column cleavage is accomplished through the use of an intein domain and any amino acid or peptide with available C-terminal thiol and amine functional groups.

Linearized relaxin is proteolytically processed to separate the A and B chain of the peptide hormone. The mixture is then subjected to oxidation-reduction conditions. In one embodiment, the A and B chain of relaxin are incubated with a mixture of oxidized and reduced glutathione for 1-48 hours at 4° C., 16° C., 20° C., 25° C., 30° C., or 37° C. In one embodiment, the A and B chain of relaxin are incubated with a mixture of cysteine and for 1-48 hours at 4° C., 16° C., 20° C., 25° C., 30° C., or 37° C.

In one aspect, the invention provides a method for producing recombinant relaxin or a variant or analogue thereof. Generally, the method comprises recombinantly expressing a fusion protein described herein in a host cell. It is noted that the host cell can be a prokaryotic or eukaryotic cell. Exemplary host cells include, but are not limited to, bacterial cells, yeast cells, plant cell, animal (including insect) or human cells. Thus, a fusion protein described herein can be expressed in a bacterial, mammalian or yeast host cell. In some preferred embodiments, the fusion protein is expressed in an E. coli cell.

Some embodiments of the various aspects described herein include a cell, e.g., a host cell. As used herein, the term “cell” refers to a single cell as well as to a population of (i.e., more than one) cells. As used herein, the cell can be a prokaryotic or eukaryotic cell. Exemplary cells include, but are not limited to, bacterial cells, yeast cells, plant cell, animal (including insect) or human cells.

In some embodiments of any one of the aspect, the cell is a host cell. The host cells can be employed in a method of producing a fusion protein described herein. Generally, the method comprises: culturing the host cell comprising a polynucleotide encoding a fusion protein described herein or a plasmid or vector comprising the polynucleotide under conditions such that the fusion protein is expressed; and optionally recovering the fusion protein from the culture medium. The fusion protein can be concentrated and purified by a variety of biochemical and chromatographic methods, including methods utilizing differences in size, charge, hydrophobicity, solubility, specific affinity, etc. between the fusion protein and other substances in the cell culture medium. In some embodiments of the various aspects described herein, the fusion protein is secreted from the host cells.

The fusion protein described herein can be produced as recombinant molecules in prokaryotic or eukaryotic host cells, such as bacteria, yeast, plant, animal (including insect) or human cell lines or in transgenic animals. Recombinant methods of producing a polypeptide through the introduction of a vector including nucleic acid encoding the polypeptide into a suitable host cell is well known in the art, such as is described in Sambrook et al., Molecular Cloning: A Laboratory Manual, 2d Ed, Vols 1 to 8, Cold Spring Harbor, N.Y. (1989); M. W. Pennington and B. M. Dunn, Methods in Molecular Biology: Peptide Synthesis Protocols, Vol 35, Humana Press, Totawa, N.J. (1994), contents of both of which are herein incorporated by reference.

The production of fusion proteins at high levels in suitable host cells requires the assembly of the polynucleotides encoding such fusion proteins into efficient transcriptional units together with suitable regulatory elements in a recombinant expression vector that can be propagated in various expression systems according to methods known to those skilled in the art. Efficient transcriptional regulatory elements could be derived from viruses having animal cells as their natural hosts or from the chromosomal DNA of animal cells. For example, promoter-enhancer combinations derived from the Simian Virus 40, adenovirus, BK polyoma virus, human cytomegalovirus, or the long terminal repeat of Rous sarcoma virus, or promoter-enhancer combinations including strongly constitutively transcribed genes in animal cells like beta-actin or GRP78 can be used. In order to achieve stable high levels of mRNA, the transcriptional unit should contain in its 3′-proximal part a DNA region encoding a transcriptional termination-polyadenylation sequence. Generally, this sequence can be derived from the Simian Virus 40 early transcriptional region, the rabbit beta-globin gene, or the human tissue plasminogen activator gene.

The vector is transfected into a suitable host cell line for expression of the fusion protein. Examples of cell lines that can be used to prepare the fusion protein described herein include, but are not limited to monkey COS-cells, mouse L-cells, mouse C127-cells, hamster BHK-21 cells, human embryonic kidney 293 cells, and hamster CHO-cells.

The expression vector encoding the fusion protein can be introduced in several different ways. For instance, the expression vectors can be created from vectors based on different animal viruses. Examples of these are vectors based on baculovirus, vaccinia virus, adenovirus, and preferably bovine papilloma virus

The transcription units encoding the corresponding DNAs can also be introduced into animal cells together with another recombinant gene, which may function as a dominant selectable marker in these cells in order to facilitate the isolation of specific cell clones, which have integrated the recombinant DNA into their genome. Examples of this type of dominant selectable marker genes are Tn5 amino glycoside phosphotransferase, conferring resistance to geneticin (G418), hygromycin phosphotransferase, conferring resistance to hygromycin, and puromycin acetyl transferase, conferring resistance to puromycin. The recombinant expression vector encoding such a selectable marker can reside either on the same vector as the one encoding the cDNA of the desired protein, or it can be encoded on a separate vector which is simultaneously introduced and integrated to the genome of the host cell, frequently resulting in a tight physical linkage between the different transcription units

Other types of selectable marker genes, which can be used together with the cDNA of the desired protein are based on various transcription units encoding dihydrofolate reductase (dhfr). After introduction of this type of gene into cells lacking endogenous dhfr-activity, preferentially CHO-cells (DUKX-B11, DG-44) it will enable these to grow in media lacking nucleosides. An example of such a medium is Ham's F12 without hypoxanthine, thymidin, and glycine. These dhfr-genes can be introduced together with the Kazal-type serine protease inhibitors' cDNA transcriptional units into CHO-cells of the above type, either linked on the same vector or on different vectors, thus creating dhfr-positive cell lines producing recombinant protein.

If the above cell lines are grown in the presence of the cytotoxic dhfr-inhibitor methotrexate, new cell lines resistant to methotrexate will emerge. These cell lines may produce recombinant protein at an increased rate due to the amplified number of linked dhfr and the desired protein's transcriptional units. When propagating these cell lines in increasing concentrations of methotrexate (1-10000 nM), new cell lines can be obtained which produce the desired protein at a very high rate.

The above cell lines producing the desired protein can be grown on a large scale, either in suspension culture or on various solid supports. Examples of these supports are micro carriers based on dextran or collagen matrices, or solid supports in the form of hollow fibers or various ceramic materials. When grown in cell suspension culture or on micro carriers the culture of the above cell lines can be performed either as a batch culture or as a perfusion culture with continuous production of conditioned medium over extended periods of time.

An example of such purification is the adsorption of the fusion protein to a monoclonal antibody or a binding peptide, which is immobilized on a solid support. After desorption, the protein can be further purified by a variety of chromatographic techniques based on the above properties.

Exemplary genera of yeast contemplated to be useful in the production of the fusion protein described herein as hosts are Pichia (formerly classified as Hansenula), Saccharomyces, Kluyveromyces, Aspergillus, Candida, Torulopsis, Torulaspora, Schizosaccharomyces, Citeromyces, Pachysolen, Zygosaccharomyces, Debaromyces, Trichoderma, Cephalosporium, Humicola, Mucor, Neurospora, Yarrowia, Metschunikowia, Rhodosporidium, Leucosporidium, Botryoascus, Sporidiobolus, Endomycopsis, and the like. Genera include those selected from the group consisting of Saccharomyces, Schizosaccharomyces, Kluyveromyces, Pichia and Torulaspora. Examples of Saccharomyces spp. are S. cerevisiae, S. italicus and S. rouxii.

Suitable promoters for S. cerevisiae include those associated with the PGKI gene, GAL1 or GAL10 genes, CYCI, PHO5, TRPI, ADHI, ADH2, the genes for glyceral-dehyde-3-phosphate dehydrogenase, hexokinase, pyruvate decarboxylase, phos-phofructokinase, triose phosphate isomerase, phosphoglucose isomerase, glucokinase, alpha-mating factor pheromone, the PRBI, the GUT2, the GPDI promoter, and hybrid promoters involving hybrids of parts of 5′ regulatory regions with parts of 5′ regulatory regions of other promoters or with upstream activation sites (e.g. the promoter of EP-A-258 067).

Convenient regulatable promoters for use in Schizosaccharomyces pombe are the thiamine-repressible promoter from the nmt gene as described by Maundrell (Maundrell K. 1990. Nmtl of fission yeast. A highly transcribed gene completely repressed by thiamine. J. Biol. Chem. 265:10857-10864) and the glucose repressible jbpl gene promoter as described by Hoffman and Winston (Hoffman C S and Winston F. 1990. Isolation and characterization of mutants constitutive for expression of the fbpl gene of Schizosaccharomyces pombe. Genetics 124:807-816).

The transcription termination signal may be the 3′ flanking sequence of a eukaryotic gene which contains proper signals for transcription termination and polyadenylation. Suitable 3′ flanking sequences may, for example, be those of the gene naturally linked to the expression control sequence used, i.e. may correspond to the promoter. Alternatively, they may be different in which case the termination signal of the S. cerevisiae ADHI gene is optionally used.

Exemplary expression systems for the production of the fusion protein described herein in bacteria include Bacillus subtilis, Bacillus brevis, Bacillus megaterium, Caulobacter crescentus, Escherichia coli BL21 and E. coli K12 and their derivatives. Convenient promoters include but are not limited to trc promoter, tac promoter, lac promoter, lambda phage promoter p_{L}, the L-arabinose inducible araBAD promoter, the L-rhamnose inducible rhaP promoter, and the anhydrotetracycline-inducible tetA promoter/operator.

In some embodiment, a polynucleotide encoding the fusion protein described herein can be fused to signal sequences which will direct the localization of a protein of the invention to particular compartments of a prokaryotic cell and/or direct the secretion of a protein of the invention from a prokaryotic cell. For example, in E. coli, one may wish to direct the expression of the protein to the periplasmic space. Examples of signal sequences or proteins (or fragments thereof) to which the proteins of the invention may be fused in order to direct the expression of the polypeptide to the periplasmic space of bacteria include, but are not limited to, the pelB signal sequence, the maltose binding protein signal sequence, the ompA signal sequence, the signal sequence of the periplasmic E. coli heat-labile enterotoxin B-subunit, and the signal sequence of alkaline phosphatase. Several vectors are commercially available for the construction of fusion proteins which will direct the localization of a protein, such as the pMAL series of vectors (New England Biolabs).

Exemplary plant systems for expression of the fusion protein described herein include tobacco, potato, rice, maize, soybean, alfalfa, tomato, lettuce and legume (summarized by Ma J K C et al. 2003. The production of recombinant pharmaceutical proteins in plants. Nat. Rev. Genet. 4:794-805). Expression of recombinant proteins in plant systems may be directed by suitable regulatory elements to specific organs or tissues such as fruits, seeds, leaves or tubers. Alternatively, proteins may be secreted from the roots. Within the cell, proteins may be targeted to particular compartments, e.g. the endoplasmic reticulum, protein bodies or plastids. There the product may accumulate to higher levels or undergo particular forms of posttranslational modification.

Exemplary examples for large-scale transgenic expression systems (for review see Pollock D P. 1999. Transgenic milk as a method for the production of recombinant antibodies. J Immunol Methods 231:147-157) include rabbit (Chrenek P et al. 2007. Expression of recombinant human factor VIII in milk of several generations of transgenic rabbits. Transgenic Res. 2007 Jan. 31), goat (Lazaris A et al. 2006. Transgenesis using nuclear transfer in goats. Methods Mol Biol. 348:213-26), pig and cattle.

In some embodiments, the method comprises separating the expressed crude fusion protein from the host cell. When the host cell excretes fusion protein, the fusion protein can be separated from the host cell by harvesting the cell culture supernatant, which contains the excreted fusion protein. When the fusion protein is expressed in the periplasmic space of a host cell, e.g., a bacterial host cell, the cell can be lysed. Methods for lysing host cells are well known in the art. Exemplary methods of lysis include, but are not limited to, mechanical, chemical, thermal, enzymatic, or a combination thereof. In some embodiments, an osmotic shock procedure can be carried out to isolate the fusion protein expressed in the periplasmic space of a bacterial expression host.

One exemplary chemical method of lysis comprises adding a non-ionic surfactant to the cell culture or cell culture supernatant comprising the host cell. The non-ionic surfactant is added to a final concentration of at least about 0.05% (w/v, w/w or v/v) or higher and allowed to mix with the cell culture or cell culture supernatant for a sufficient period of time to lyse host cells present in the cell culture or cell culture supernatant. For example, the non-ionic surfactant is mixed with the cell culture or cell culture supernatant for a period of from about 15 minutes to about 2 hours. The mixing can be at ambient temperature or an elevated temperature. For example, the mixing with the non-ionic surfactant can be at a temperature from about 15° C. to about 37° C.

Exemplary non-ionic surfactants and classes of non-ionic surfactants for lysing host cells can include polyarylphenol polyethoxy ethers; polyalkylphenol polyethoxy ethers; polyglycol ether derivatives of saturated fatty acids; polyglycol ether derivatives of unsaturated fatty acids; polyglycol ether derivatives of aliphatic alcohols; polyglycol ether derivatives of cycloaliphatic alcohols; fatty acid esters of polyoxyethylene sorbitan; alkoxylated vegetable oils; alkoxylated acetylenic dials; polyalkoxylated alkylphenols; fatty acid alkoxylates; sorbitan alkoxylates; sorbitol esters; C₈ to C22 alkyl or alkenyl polyglycosides; polyalkoxy styrylaryl ethers; alkylamine oxides; block copolymer ethers; polyalkoxylated fatty glyceride; polyalkylene glycol ethers; linear aliphatic or aromatic polyesters; organo silicones; polyaryl phenols; sorbitol ester alkoxylates; and mono- and diesters of ethylene glycol and mixtures thereof; ethoxylated tristyrylphenol; ethoxylated fatty alcohol; ethoxylated lauryl alcohol; ethoxylated castor oil; and ethoxylated nonylphenol; alkoxylated alcohols, amines or acids.

### 1^{st} Affinity Purification

The cell culture or cell culture supernatant may comprise impurities, e.g., cellular material. Therefore, the method can comprise a post-lysis step of removing or reducing amount of impurities from the cell culture or cell culture supernatant. For example, the crude fusion protein can be further separated from cellular material, for example, via centrifugation or affinity purification.

Accordingly, in some embodiments, the method comprises a step of isolating the released fusion protein prior to cleaving the protease cleavable domain. For example, the step comprises affinity chromatography using an affinity tag present in the fusion protein prior to cleaving the protease cleavable domain. In some embodiments, the step of isolating the released fusion protein prior to cleaving the protease cleavable domain comprises affinity purification using the first affinity tag of the fusion protein.

Generally, the step comprises contacting a preparation the crude fusion protein with an affinity chromatography media under conditions that allow binding of fusion protein to the affinity chromatography media via one of the affinity tags. The bound fusion protein is eluted from the affinity chromatography media using an elution buffer and recovering an eluate, comprising the fusion protein. In some embodiments, immobilized metal ion affinity chromatography (IMAC) is employed for this step.

Immobilized metal ion affinity chromatography is a versatile separation procedure that exploits differences in the affinities exhibited by many biopolymers for metal ions. The technique involves the chelation of a suitable metal ion onto a solid support matrix whose surface has previously been chemically modified with a polydentate ligand. The resulting immobilized metal ion chelating complex then has the potential to coordinate with one or more electron donor groups resident on the surface of the interacting protein (Sulkowski, E., Trends in Biotechnology, 3 (1985) 1-6; Porath, J., Carlsson, I., Olsson, I. and Belfrage, G., Nature, 258 (1975) 598-599; Kagedal, L., in “Protein Purification” (Ed., J. C. Janson, and L. Ryden), VCH Publishers (1989) pp. 227-251; Zachariou, M. and Hearn, M. T. W., Biochemistry, 35 (1996) 202-211. Separation selectivity is then achieved on the basis of differences in the thermodynamic stabilities of the adsorbed protein/immobilized metal ion complexes. Proteins whose adsorption complexes are the least stable will be eluted first, whilst proteins that form more stable complexes will be eluted later. The greater the difference in the equilibrium association constants, i.e. the larger the differences in the dissociation constants (K_{D}) of the respective protein/immobilized metal ion coordination complexes, the higher the resolution obtained. Consequently, the amino acid composition, surface distribution of particular amino acid residues, as well as the conformation of the protein all play important roles in determining the affinity of a protein for a particular IMAC system. As a result, proteins with very similar properties with respect to charge, molecular size and amino acid composition, but with differences in their tertiary structures, may be resolved.

In some embodiments, the affinity chromatography media for the metal ion affinity chromatography comprises cobalt ions bound to cross-linked agarose.

### Cleavage of the Cleavable Domain

Some embodiments of the method described herein comprises a step of cleaving the protease cleavable domain of the fusion protein, e.g., to remove the solubility domain. A skilled artisan would appreciate that a protease cleavable domain described herein encompasses an amino acid sequence of a cleavage site for a protease. Cleavage site sequence for proteases are well known in the art and amenable to the present invention. Some exemplary protease cleavage site sequences include but are not limited to, QXXYXES (SEQ ID NO: 1), QXXYFXG (SEQ ID NO: 2), DDDDK (SEQ ID NO: 3) OR RGYZ (SEQ ID NO: 4), where X is any amino acids, Y is a negatively charged amino acid and Z is a cationic or neutral amino acid.

In some embodiments, the protease cleavable domain comprises a cleavage site of a protease selected from the group consisting of potyvirus Ma proteases, potyvirus HC proteases, potyvirus P1 (P35) proteases, byovirus Ma proteases, byovirus RNA-2-encoded proteases, aphthovirus L proteases, enterovirus 2A proteases, rhinovirus 2A proteases, picorna 3C proteases, comovirus 24K proteases, nepovirus 24K proteases, rice tungro spherical virus (RTSV) 3C-like protease, parsnip yellow fleck virus (PYVF) 3C-like protease, heparin, thrombin, factor Xa, PreScission protease, subtilisins (including PC2, PC1/PC3, PACE4, PC4, PC5/PC6, LPC/PC7IPC8/SPC7 and SKI-1), chemotrypsin and enterokinase. Exemplary sequences for cleavage site of a protease include, but are not limited to, Tobacco etch virus (TEV) protease, EXXYXQ-(G/S) (SEQ ID NO: 5); tobacco vein mottling virus (TVMV) protease, GTVRFQ-(G/S) (SEQ ID NO: 6); furin, RXR/K-R (SEQ ID NO: 7); VP4 of IPNV, S/TXA-S/AG (SEQ ID NO: 8); 3C protease of rhinovirus, LEVLFQ-GP (SEQ ID NO: 9); enterokinase, DDDDK-X (SEQ ID NO: 10) and (D/E)R-M (SEQ ID NO: 11); thrombin, LVPR-GS (SEQ ID NO: 12); MMP, PLGLAG (SEQ ID NO: 13); Factor Xa protease, IE/DGR-X (SEQ ID NO: 14); genenase I, PGAAH-Y (SEQ ID NO: 15); KEX2 protease, MYKR-EAD (SEQ ID NO: 16); Granzyme B, IEPD-X (SEQ ID NO: 17); and Caspase-3, DEVD-X (SEQ ID NO: 18), where X is any amino acid. In some embodiments, the protease cleavable domain comprises the amino acid sequence GPLGMLSQ (SEQ ID NO: 19), GPLGLWAQ (SEQ ID NO: 20), GPLGLAG (SEQ ID NO: 21), KKNPAELIGPVD (SEQ ID NO: 22), KKQPAANLVAPED (SEQ ID NO: 23), ENLYFQG (SEQ ID NO: 24) or ENLYFQS (SEQ ID NO: 25).

In some preferred embodiments, the protease cleavable domain comprises a cleavage site of TEV, e.g., EXXYXQ(G/S) (SEQ ID NO: 5), wherein X represents any amino acid (cleavage by TEV occurs between Q and G or Q and S). In a specific preferred embodiment, the protease cleavable domain comprises a cleavage site with amino acid sequence ENLYFQG (SEQ ID NO: 24).

Generally, the protease cleavable domain is positioned between the relaxin domain and the solubility domain. Without limitations, the protease cleavable domain can be positioned at the N-terminus or C-terminus of the relaxin domain. In some preferred embodiments, the protease cleavable domain is at the N-terminus of the relaxin domain.

Generally, the step of cleaving the protease cleavable domain comprises contacting the fusion protein with a protease under conditions appropriate for cleavage to take place.

### Solubility Domain

In some embodiments of the various aspects described herein, the fusion protein comprises a solubility domain. As used herein, “solubility domain” refers to an amino sequence that, upon fusion with a target polypeptide, can facilitate the folding of the target polypeptide and increase the solubility of the fusion protein. Various “solubility domains” are well known in the art. Suitable solubility-domains that can be used in the invention include, but not limit to, maltose binding protein (MBP), glutathione 5-transferase (GST), thioredoxin (TRX), NusA, SUMO, DsbC, Z, GB1, MBP and T7PK. In a preferred embodiment, the solubility domain is MBP. In some preferred embodiments, the solubility domain comprises the amino acid sequence SEQ ID NO: 26

Without limitations, the solubility domain can be linked to the N-terminus or C-terminus of the relaxin domain. In some preferred embodiments, the solubility domain is linked to the N-terminus of the relaxin domain. For example, the solubility domain is linked to the N-terminus of the relaxin domain via the protease cleavable domain.

### Self-Cleaving Domain

Some embodiments of the method described herein comprises a step of cleaving the self-cleaving domain of the cleaved fusion protein portion comprising the relaxin domain. A skilled artisan would appreciate that a self-cleaving domain described herein encompasses an amino acid sequence that can be induced to auto-lyse under the appropriate conditions. In some embodiments of any one of the aspects, the self-cleaving domain comprises an intein.

As used herein, “intein” refers to an auto-catalytic domain of the Hog/INTein (Hint) superfamily that splices itself out of a polypeptide by forming a peptide bond between two flanking polypeptides. As used herein, “intein” includes naturally-occurring inteins, or functional mutants or variants thereof, including engineered and synthetic inteins. Exemplary inteins include, but are not limited to DnaB helicase (dnaB) inteins, DNA polymerase III a subunit (dnaE) inteins, DNA polymerase III i subunit (dnaX) inteins, RecA inteins, DNA gyrase subunit A inteins (gyrA), and DNA gyrase subunit B inteins (gyrB), including functional and mutants and modifications thereof, such as mini-inteins, N-terminal and/or C-terminal mutants, and the like. While inteins are known to vary in length and sequence, a feature characteristic of many inteins is a Ser (S) or Cys (C) on the N terminus, and a C terminal motif of either His-Asn-Cys (HNC) or His-Asn-Ser (HNS), and some of these N and C terminal motifs have been shown to function in splicing and/or cleavage activity of the intein. One or more amino acid residues near the N terminus or C terminus of the intein can be mutated to reduce or eliminate splicing activity. For example, one or more amino acid residues near the N terminus or C terminus of the intein can be modified such that N terminal cleavage, and/or C terminal cleavage is increased. Modifying the most C-terminal residue of an intein, eliminates splicing activity of the intein and C terminal cleavage, while preserving the intein's N-terminal cleavage activity. Mutating the most N-terminal residue of an intein eliminates splicing activity and N terminal cleavage while preserving the intein's C terminal cleavage. Intein autolysis can be induced by adjusting the temperature, pH, salt concentration and/or mercapto group concentration.

In some embodiments of any one of the aspects, the intein is selected from the group consisting of Mxe GyrA intein, Ssp DnaB mini-intein, Mth RIR1 intein and Sce VMA1 intein. In some preferred embodiments, the intein is Mxe GyrA intein. In some preferred embodiments, the intein domain comprises the amino acid sequence SEQ ID NO: 27.

It is noted that an intein can be a C-terminal cleavable inteins of target proteins such as Mxe GyrA intein and Mth RIR1 intein. Alternatively, the intein can be an N-terminal cleavable intein of the target protein, such as Ssp DnaB mini intein. C-terminal cleavable inteins are sensitive to temperature and pH values. The intein can be effectively degraded when the temperature rises to 25° C. or the pH decreases from 8.5 to 6.0. Since the change in pH value is small, the occurrence of protein denaturation caused by pH change can be avoided. In some embodiments, N-terminal cleavable inteins are preferred because mercapto compounds require inducers for degradation and can therefore be effectively controlled.

Inteins particularly suitable for the present invention include N-terminal cleavable Ssp DnaB mini-intein, which can autolyze at pH 6.0-7.0 and/or 18-25° C.; C-terminal cleavable Mth RIR1 intein and Mxe GyrA intein, which can autolyze at pH 8.5 in the presence of 40 mM mercapto groups. Details for other inteins can be found in the Intein database of NEB at the website neb. com/neb/inteins.html.

The lysis solution to be added for inducing the autolysis of Interin may be water, a buffer solution, or an aqueous solution containing mercapto compounds. A skilled artisan may reasonably select a lysis solution based on the Intein to be used. For example, for Ssp DnaB mini-Intein, a preferred lysis solution is 20 mM Tris-Cl, 500 mM NaCl and 1 mM EDTA, pH 6.0-7.0. For Mth RIR1 Intein, a preferred lysis solution is 20 mM Tris-HCl, 500 mM NaCl, 1 mM EDTA and 40 mM DTT, pH8.0-8.5.

In some embodiments, the rearrangement and resulting cleavage of the self-cleavable domain is precipitated by the addition of a conjugate-ligand. In some specific embodiments, the product after rearrangement results in the creation of a fusion construct containing the relaxin domain and the conjugate-ligand linked by a peptide bond.

Generally, the self-cleavable domain is positioned between the relaxin domain and one of the affinity tags. Without limitations, the self-cleavable domain can be positioned at the N-terminus or C-terminus of the relaxin domain. In some preferred embodiments, the self-cleavable domain is at the C-terminus of the relaxin domain.

In some embodiments, the self-cleaving domain, e.g., intein comprising fusion protein is contacted with an affinity chromatography media under conditions that allow binding of fusion protein to the affinity chromatography media via one of the affinity tags, e.g., the affinity tag closest to the self-cleaving domain. Intein autolysis induced by adjusting the temperature, pH, salt concentration and/or conjugate-ligand concentration. Adsorption of the affinity tag is maintained and thus purification of the cleaved portion of the fusion protein, i.e., the fragment comprising the relaxin domain, with or without the conjugate ligand, is accomplished.

### Relaxin Domain

Embodiments of the various aspects described herein include a relaxin domain. As used herein, “relaxin domain” refers to a domain comprising relaxin or variants or analogues thereof. The terms “relaxin” or “relaxin-2” as used herein, refer to a polypeptide belonging to the relaxin family (e.g., relaxin-2), a relaxin analog (e.g., a polypeptide that binds to a relaxin receptor), or a fragment (e.g., a bioactive fragment) or variant of any of the foregoing and/or any agent that is an agonist of an agent that binds the relaxin receptor family of proteins (RXFP1, RXFP2, RXFP3, RXFP4).

Relaxin is an approximately 6-kDa protein belonging to the insulin superfamily (Sherwood O.D., Endocr. Rev. 2004, 25(2):205-34). Like insulin, relaxin is processed from a prepro-form to the mature hormone, containing A and B peptide chains connected by two interchain disulfide bridges and one intrachain disulfide within the A chain (Chan L. J. et al., Protein Pept. Lett. 2011, 18(3):220-9). Relaxin readily decreases collagen secretion and increases collagen degradation by increasing the expression of MMPs and decreasing the expression of TIMPs (Samuel C. S. et al., Cell Mol. Life Sci. 2007, 64(12):1539-57). This hormone is involved in reproduction, where it inhibits uterine contraction and induces growth and softening of the cervix to assist child delivery (Parry L. J. et al., Adv. Exp. Med. Biol. 2007, 612:34-48). Recently, a highly purified recombinant form of H2 relaxin, or human relaxin-2, has been tested in a number of in vitro and in vivo systems to evaluate both its ability to modify connective tissue and its potential antifibrotic properties. Several studies report that relaxin-2 acts at multiple levels to inhibit fibrogenesis and collagen overexpression associated with fibrosis and is able to prevent and treat pulmonary, renal, cardiac, and hepatic fibrosis (Bennett R. G., Transl. Res. 2009, 154(1):1-6). Relaxin treatment of human fibroblasts caused a reduction in levels of collagen types I and III and fibronectin (Unemori E. N. et al., The Journal of Clinical Investigation 1996, 98(12):2739-45). In vivo, relaxin-2 decreased collagen build-up in the lung induced by bleomycin and improved the overall amount of fibrosis (Unemori E. N. et al., The Journal of Clinical Investigation 1996, 98(12):2739-45). In cultured renal fibroblasts, epithelial cells and mesangial cells, relaxin-2 decreased TGF-β-induced fibronectin levels and increased fibronectin degradation (McDonald G. A. et al., American Journal of Physiology Renal Physiology 2003, 285(1):F59-67). Unless specified to the contrary, the terms “relaxin” or “relaxin-2” as used herein encompasses a relaxin or an analog, a fragment (e.g., a bioactive fragment) or a variant thereof. The term “relaxin or an analog, a fragment or a variant thereof” encompasses any member of the relaxin-like peptide family which belongs to the insulin superfamily. The relaxin-like peptide family includes relaxin-like (RLN) peptides, e.g., relaxin-1 (RLN1), relaxin-2 (RLN2) and relaxin-3 (RLN3), and the insulin-like (INSL) peptides, e.g., INSL3, INSL4, INSL5 and INSL6. Representative sequences of human RLN1 are listed herein as SEQ ID NOS: 28-31; representative sequences of human RLN2 are listed herein as SEQ ID NOS: 32-34; representative sequences of human RLN3 are listed herein as SEQ ID NOS: 35-37; a representative sequence of human INSL3 is listed herein as SEQ ID NO: 38; representative sequences of human INSL4 are listed herein as SEQ ID NOS: 39-40; representative sequences of human INSL5 are listed herein as SEQ ID NOS: 41-42; and a representative sequence of human INSL6 is listed herein as SEQ ID NO: 43. In some embodiments, the term “relaxin or an analog, a fragment or a variant thereof may encompass any polypeptide having at least 70%, e.g., at least 75%, at least 80%, at least 85%, at least 90%, at least 95%, 96%, 97%, 98% or at least 99% sequence identity with any of SEQ ID NOS: 28-43, as well as any polypeptide sequence that comprises any of SEQ ID NOS: 28-43. In one embodiment of the invention, the relaxin includes RLN1, RLN2 or RLN3. In one embodiment, the relaxin is relaxin-1. In another embodiment, the relaxin is relaxin-3. In a preferred embodiment, the relaxin is relaxin-2. In another embodiment of the invention, the relaxin includes INSL3, INSL4, INSL5 or INSL6. In one embodiment, the relaxin is INSL3. In one embodiment, the relaxin is INSL4. In one embodiment, the relaxin is INSL5. In one embodiment, the relaxin is INSL6.

In some embodiments, the relaxin is recombinantly produced, for example in a bacterial, mammalian or yeast host cell. In other aspects the relaxin has been fully or partially chemically synthesized.

In some embodiments, the term relaxin encompasses any natural, synthetic, or semi-synthetic composition that is capable of interacting with a relaxin family protein receptors (RXFP1, RXFP2, RXFP3, RXPF4) that impacts the form, function, or activity of the receptor. These compounds include but are not limited to native relaxin-2, relaxin-2 variants, polypeptides, DNA or RNA polynucleotides, small molecules, as well as any of the previously listed compounds conjugated to, or associated with, the relaxin-2 protein.

The term “relaxin or an analog, a fragment or a variant thereof” may also encompass any member of the relaxin-like peptide family, e.g., relaxin-1 (RLN1), relaxin-2 (RLN2) and relaxin-3 (RLN3), and the insulin-like (INSL) peptides, e.g., INSL3, INSL4, INSL5 and INSL6. Representative sequences of human RLN1 are listed herein as SEQ ID NOS: 28-31; representative sequences of human RLN2 are listed herein as SEQ ID NOS: 32-34; representative sequences of human RLN3 are listed herein as SEQ ID NOS: 35-37; representative sequence of human INSL3 is listed herein as SEQ ID NO: 38; representative sequences of human INSL4 are listed herein as SEQ ID NOS: 39-40 representative sequences of human INSL5 are listed herein as SEQ ID NOS. 41-42; and representative sequence of human INSL6 is listed herein as SEQ ID NO: 43. The term “relaxin or an analog SEQ ID NOS: 39-40, a fragment or a variant thereof” also in some embodiments encompasses any polypeptide having at least 70%, e.g., at least 75%, at least 80%, at least 85%, at least 90%, at least 95% or at least 99% sequence identity with any of SEQ ID NOS: 28-43, as well as any polypeptide sequence that comprises any of SEQ ID NOS: 28-43. In one embodiment of the formulation, the relaxin includes RLN1, RLN2 or RLN3. In one embodiment, the relaxin is relaxin-2. In another embodiment, the relaxin includes INSL3, INSL4, NSL5 or INSL6.

The term “relaxin or an analog, a fragment or a variant thereof” also in some embodiments may encompass any mutant member of the relaxin-like peptide family. Such mutant may be, e.g., a RLN1, RLN2, RLN3, INSL3, INSL4, INSL5 or INSL6 comprising one or more mutations, e.g., substitutions, additions or deletions of one or more amino acids (native or non-native) in the known sequence of RLN1, RLN2, RLN3, INSL3, INSL4, INSL5 or INSL6. For example, a mutant member of the relaxin-like peptide family may comprise any naturally occurring or artificially produced variants of RLN1, RLN2, RLN3, INSL3, INSL4, INSL5 or INSL6.

The term “relaxin fragment” or “a fragment of relaxin” as used herein encompasses a fragment of a relaxin, i.e., a partial sequence of any member of the relaxin-like peptide family, that retains its ability to treat stiffened joints through interaction with the relaxin family receptors. Examples include those sequences described in European Patent Office Application No. EP1641824B1 (Relaxin superfamily peptide analogues), the entire contents of which are incorporated herein by reference.

The term “relaxin analog” or an “analog of relaxin” includes any non-relaxin polypeptide sequence that possesses the biological activity of relaxin, i.e., the ability to interact with the relaxin family receptors. In one embodiment, such polypeptide sequence may comprise prolactin or an analog, a fragment or a variant thereof. In another embodiment, such sequence may comprise the truncated B-chain analogue of relaxin known as B7-33, described in ACS Appl. Mater. Interfaces 2019, 11, 49, 45511-45519.

In some embodiments, the term agent or “relaxin analog” also includes a relaxin receptor agonist, e.g., any agent, such as a small molecule, a polypeptide, a polynucleotide or a polysaccharide, that can bind to and activate a relaxin receptor, e.g., one or more of RXFP1, RXFP2, RXFP3 and RXFP4. For example, a relaxin receptor agonist may be a polypeptide comprising the receptor binding site of relaxin. A relaxin receptor agonist may also be a polypeptide comprising any other sequence capable of binding to and activating the relaxin receptor, e.g., RXFP1, RXFP2, RXFP3 and RXFP4. Other examples include those agonists described in US Patent Application No. US20130237481A1 (Modified relaxin polypeptides and their uses), US Patent Application No. US20180222960A1 (Modified relaxin polypeptides comprising a pharmacokinetic enhancer and uses thereof), U.S. Pat. No. 8,445,635B2 (Modified H2 relaxin for tumor suppression), European Patent Office Application No. EP3067365A1 (Human relaxin analogue, pharmaceutical composition of same, and pharmaceutical application of same), and US Patent Application No. US20180222960A1 (Modified relaxin polypeptides comprising a pharmacokinetic enhancer and uses thereof) the entire contents of which are incorporated herein by reference.

The term “relaxin or an analog, a fragment or a variant thereof” includes any recombinantly produced relaxin, such as, e.g., Serelaxin (RLX030) developed by Novartis. Methods for producing recombinant relaxin, e.g., relaxin-2, are described, .e.g., in U.S. Pat. No. 5,464,756, the entire contents of which are incorporated herein by reference. The recombinantly produced relaxin or analog, fragment or variant thereof may comprise a relaxin sequence, e.g., RLN1, RLN2, RLN3, INSL3, INSL4, INSL5 or INSL6, and a histidine (His) tag to aid in the purification of the relaxin after being recombinantly produced.

The relaxin or analog, fragment or variant thereof may also comprise one or more chemical modifications, e.g., chemical groups covalently attached to the relaxin or an analog, a fragment or a variant thereof. Such chemical groups may include, e.g., carbohydrates or other polymers, e.g., polyethylene glycol (PEG), e.g., polypeptide, e.g. one or more lipids (Design and Synthesis of Potent, Long-Acting Lipidated Relaxin-2 Analogs, Bioconjugate Chem. 2019, 30, 1, 83-89). Other examples include fragments or variants described in US Patent Application No. US2018/0326079 (NOVELFATTYACIDS AND THEIR USE IN CONJUGATION TO BIOMOLECULES), U.S. Pat. No. 9,931,372B2 (SYNTHETIC APELIN FATTYACID CONJUGATES WITH IMPROVED HALF-LIFE), the entire contents of which are incorporated herein by reference.

In some embodiments, the term relaxin includes relaxin attached, e.g., covalently attached, to an immunoglobulin or a fragment of an immunoglobulin, e.g., an antibody or a fragment of an antibody, for example, the immunoglobulin fusion proteins described in WO 2017/100540. In other embodiments, the term relaxin does not include relaxin attached, e.g., covalently attached, to an immunoglobulin or a fragment of an immunoglobulin.

In some embodiments, the relaxin domain released after autolysis of the self-cleaving domain can be cleaved to separate the A and B chains. Generally, the step of cleaving the relaxin domain to separate the A and B chains comprises incubating the relaxin domain comprising fragment of the fusion protein with a protease. For example, the step of cleaving the released relaxin domain comprises incubating the released relaxin domain with transmembrane serine protease, e.g., a type II transmembrane serine protease (TTSP). In some embodiments of any one of the aspects, the TTSP is an enteropeptidase (enterokinase). In some embodiments, the cleavage of the A and B chains is accomplished through use of bovine enterokinase (light chain).

The released A and B chains can be combined to produce biologically active relaxin or a variant or analogue thereof. Generally, the step of combining the released A and B chains to produce biologically active relaxin or a variant or analogue thereof comprises exposing the A and B chains to oxidation-reduction conditions. For example, the step of combining the released A and B chains comprises reducing the A and B chains at a pH of from about 7.0 to 12 under exposure to oxygen, under conditions whereby the B-chain, but not the relaxin product, is denatured. In some embodiments, the step of combining the released A and B chains is carried out in an aqueous medium. The pH of the medium ranges from about 7.0 to about 12. For example, the pH will be from about 7.5 to about 11.0. Preferably, the pH is maintained within the range of from about 8.0 to about 9.5. This step can be performed at a temperature from about 4° C. to about 37° C. For example, this step can be performed at a temperature of 4° C., 16° C., 20° C., 25° C., 30° C., or 37° C. The incubation can be from about 1 hour to 48 hours.

In some embodiments of any one of the aspects, the released A and B chains are incubated with a mixture of oxidized and reduced glutathione for a period of about 1 hour to about 48 hours at a temperature from about 4° C. to about 37° C. In some embodiments, the released A and B chains are incubated cysteine for a period of about 1 hour to about 48 hours at a temperature from about 4° C. to about 37° C.

In other embodiments, the released relaxin domain is a single polypeptide chain, wherein the A and B chains are linked together by a polypeptide linker which does not contain a protease cleavage site. In some embodiments, the linker linking the A and B chains comprises the amino acid sequence selected from SEQ ID NOS: 44-50.

The produced relaxin or a variant or analogue thereof can be further isolated or purified by any of a wide variety of methods known in the art for protein isolation/purification. Exemplary techniques for protein isolation/purification include chromatographic techniques. These can include gel filtration, ion-exchange chromatography, affinity chromatography, reverse phase HPLC, etc . . .

In some embodiments of any one of the aspects, the relaxin or a variant or analogue thereof produced by the method described herein at least 50%, 55%, 60%, 65%, 70%, 75%, 80%, 85%, 90%, or 95% pure by chromatography.

### Affinity Tags

in some embodiments of the various aspects described herein, the fusion protein comprises one or more epitopes or affinity tags. Without wishing to be bound a theory, the epi tope of affinity tag can provide a convenient means for isolating or purifying the fusion protein. When present, the epitope or affinity tag can be located anywhere in the fusion protein. For example, the epitope or affinity tag can be at the N-terminal, C-terminal or at an internal position of the polypeptide. In some embodiments, an epitope or affinity tag is at a position N-terminal of the solubility domain. In some embodiments, an epitope or affinity tag is at a position C-terminal of the self-cleaving domain.

A number of epitope or affinity tags are known in the art. These are usually divided into 3 classes according to their size: small tags have a maximum of 12 amino acids, medium-sized ones have a maximum of 60 and large ones have more than 60. The small tags include the Arg-tag, the His-tag, the avidin biotin, or streptavidin (Strep)-tag, the Flag-tag, the T7-tag, the V5-peptide-tag and the c-Myc-tag, the medium-sized ones include the S-tag, the HAT-tag, the calmodulin-binding peptide, the chitin-binding domain (CBD) and some cellulose-binding domains. The latter can contain up to 189 amino acids and are then regarded, like the glutathione-S-transferase (GST)-and maltose binding protein (MBP)-tag, as large affinity tags.

Some exemplary affinity tag sequences include, but are not limited to, 6-HIS tag (HHHHHH (SEQ ID NO: 51), HA tag (YPYDVPDYA, SEQ ID NO: 52), ac-Myc epitope (EQKLISEEDL, SEQ ID NO: 53), AU1 tag (DTYRYI, SEQ ID NO: 54), Flag-tag (DYKDDDDK, SEQ ID NO: 55). In some preferred embodiments, the chitin binding domain comprises the amino acid sequence of SEQ ID NO: 56.

The methodology described herein takes advantages of multiple isolation/purification steps. For example, a first affinity tag can be used for a first isolation/purification step and a second affinity tag can be used for a second isolation/purification step. Accordingly, in some embodiments, the fusion protein comprises two affinity tags, which can be the same or different. For example, the fusion protein can comprise two different affinity tags. In some embodiments, the fusion protein comprises a first affinity tag at N-terminal of the fusion protein and a second affinity tag at C-terminal of the fusion protein. In some embodiments, a first epitope or affinity tag is at a position N-terminal of the solubility domain and a second epitope or affinity tag is at a position C-terminal of the self-cleaving domain.

In some embodiments, the fusion protein comprises a HIS-tag and a CBD domain. For example, the fusion protein comprises a HIS-tag at N-terminal of the fusion protein and a CBD domain at C-terminal of the fusion protein. In some embodiments, the HIS-tag is at a position N-terminal of the solubility domain and the CBD tag is at a position C-terminal of the self-cleaving domain.

### Linkers

In some embodiments of any of the aspects, the fusion protein can comprise a linker between two domains of the fusion protein. In other embodiments of any of the aspets, the fusion protein can comprise a linker inside of any of the fusion protein domains. In a specific embodiment, a flexible, non-cleavable linker exists between the A chain and B chain of the relaxin domain. The linker can be a chemical linker, a single peptide bond (e.g., linked directly to each other) or a peptide linker containing one or more amino acid residues (e.g. with an intervening amino acid or amino acid sequence between the first and second domains).

In some embodiments of any of the aspects, the linker used to link two domains is a flexible linker. As used herein, a “flexible linker” is a linker which does not have a fixed structure (secondary or tertiary structure) in solution and is therefore free to adopt a variety of conformations. Generally, a flexible linker has a plurality of freely rotating bonds along its backbone. In contrast, a rigid linker is a linker which adopts a relatively well-defined conformation when in solution. Rigid linkers are therefore those which have a particular secondary and/or tertiary structure in solution.

In some embodiments of the various aspects described herein, the linker is a peptide linker. The term “peptide linker” as used herein denotes a peptide with amino acid sequences, which is in some embodiments of synthetic origin. It is noted that peptide linkers may affect folding of a given fusion protein, and may also react/bind with other proteins, and these properties can be screened for by known techniques. A peptide linker can comprise 1 amino acid or more, 5 amino acids or more, 10 amino acids or more, 15 amino acids or more, 20 amino acids or more, 25 amino acids or more, 30 amino acids or more, 35 amino acids or more, 40 amino acids or more, 45 amino acids or more, 50 amino acids or more and beyond. A peptide linker can comprise about 7 amino acids, about 8 amino acids, about 9 amino acids, about 10 amino acids, about 11 amino acids, about 15 amino acids, or about 16 amino acids. Conversely, a peptide linker can comprise less than 50 amino acids, less than 45 amino acids, less than 40 amino acids, less than 35 amino acids, less than 30 amino acids, less than 30 amino acids, less than 25 amino acids, less than 20 amino acids, less than 15 amino acids or less than 10 amino acids. In some embodiments of the various aspects described herein, the peptide linker comprises from about 5 amino acids to about 40 amino acids. For example, the peptide linker can comprise from about 5 amino acids to about 35 amino acids, from about 10 amino acids to 30 amino acids, or from about 10 amino acids to about 25 amino acids.

In some embodiments, the relaxin domain contains a peptide linker, wherein the A chain and B chains are linked together by a non-cleavable linker resulting in a single polypeptide chain. In preferred embodiments, the linker linking the A chain and B chain comprises the amino acid sequence selected from SEQ ID NOS: 44-50.

In some embodiments of the various aspects described herein, the linker comprises 10, 11, 12, 13, 14, 15, 16, 17, 18, 19, 20, 21, 22, 23, 24 or 25 amino acids. For example, the linker comprises 10, 11, 12, 13, 14, 15, 16, 17, 18, 19 or 20 amino acids. Preferably, the linker comprises 12, 13, 14, 15, 16, 17 or 18 amino acids. More preferably, the linker comprises 14, 15 or 16 amino acids. In some embodiments of the various aspects described herein, the linker comprises 15 amino acids.

Some exemplary peptide linkers include those that consist of glycine and serine residues, the so-called Gly-Ser polypeptide linkers. As used herein, the term “Gly-Ser polypeptide linker” refers to a peptide that consists of glycine and serine residues. In some embodiments of the various aspects described herein, the peptide linker comprises the amino acid sequence (GlyₓSer)ₙ where x is 2, 3, 4, 5 or 6, and n is 1, 2, 3, 4, 5, 6, 7, 8, 9 or 10 (SEQ ID NO: 57). In some preferred embodiments, x is 4. In some preferred embodiments, n is 2. In some embodiments of the various aspects described herein, x is 3 and n is 3, 4, 5 or 6. In some embodiments of the various aspects described herein, x is 3 and n is 4 or 5. In some embodiments of the various aspects described herein, x is 4 and n is 3, 4, 5 or 6. In some embodiments of the various aspects described herein, x is 4 and n is 4 or 5. In some embodiments of the various aspects described herein, x is 3 and n is 2. In some embodiments of the various aspects described herein, x is 3 or 4 and n is 1.

More exemplary linkers, in addition to those described herein, include a string of histidine residues, e.g., His6 (HHHHH, SEQ ID NO: 51); sequences made up of Ala and Pro, varying the number of Ala-Pro pairs to modulate the flexibility of the linker; and sequences made up of charged amino acid residues e.g., mixing Glu and Lys. Flexibility can be controlled by the types and numbers of residues in the linker. See, e.g., Perham, 30 Biochem. 8501 (1991); Wriggers et al., 80 Biopolymers 736 (2005).

In some embodiments of the various aspects described herein, the linker can be a chemical linker. Chemical linkers can comprise a direct bond or an atom such as oxygen or sulfur, a unit such as NH, C(O), C(O)NH, SO, SO₂, SO₂NH, or a chain of atoms, such as substituted or unsubstituted C₁-C₆ alkyl, substituted or unsubstituted C₂-C₆ alkenyl, substituted or unsubstituted C₂-C₆ alkynyl, substituted or unsubstituted C₆-C₁₂ aryl, substituted or unsubstituted C₅-C₁₂ heteroaryl, substituted or unsubstituted C₅-C₁₂ heterocyclyl, substituted or unsubstituted C₃-C₁₂ cycloalkyl, where one or more methylenes can be interrupted or terminated by O, S, S(O), SO₂, NH, or C(O).

In some embodiments of any one of the aspects, a linker used to link two domains can be a cleavable linker. For example, the linker comprises a cleavable group. A cleavable group is one which is sufficiently stable under a first set of conditions and can be cleaved to release the two parts the cleavable group is holding together. In a preferred embodiment, the cleavable group is cleaved at least 10 times or more, preferably at least 100 times faster under a first reference condition than under a second reference condition. Cleavable groups are susceptible to cleavage agents, e.g., pH, redox potential or the presence of degradative molecules. In some embodiments, the linker comprises a peptide based cleavable group comprising two or more amino acids. In some embodiments, the peptide-based cleavable group comprises the amino acid sequence that is the substrate for a peptidase or a protease.

### Methods for Treating Fibrotic Diseases

Also provided herein is a method for treating a fibrotic disease. Generally, the method comprises administering to a subject in need thereof a recombinantly produced relaxin, e.g., relaxin-2 or a variant or analogue described herein.

In some embodiments of any one of the aspects, the fibrotic disease is selected from the group consisting of idiopathic pulmonary fibrosis, cystic fibrosis, hypertension, hepatitis B or C, non-alcoholic steatohepatitis, non-alcoholic fatty liver disease, Cholestasis, autoimmune hepatitis cirrhosis, chronic kidney disease, end-stage renal disease, renal interstitial fibrosis, heart failure, myocardial infarction, aortic stenosis, hypertrophic cardiomyopathy, Crohn's disease, inflammatory bowel disease, enteropathies, other intestinal fibrosis, scleroderma, keloids, hypertrophic scars, cellulite, Peyronie's disease, uterine fibroids, Congenital Fibrosis of the Extraocular Muscles, subretinal fibrosis, epiretinal fibrosis, and corneal fibrosis.

In some embodiments of anyone of the aspect described herein, the fibrotic disease is selected from the group consisting of Duchenne Muscular Dystrophy, Becker Muscular Dystrophy, Spinal Muscular Atrophy (Type I, II, III, or IV), Cerebral Palsy, Stroke, Traumatic Brain Injury, peripheral nerve injury, Arthrogryposis Multiplex Congenita, fibrosis of the humeroradial joint, fibrosis of the humeroulnar joint, fibrosis of the glenohumeral joint, fibrosis of the tibiofemoral joint, fibrosis of the acetabulofemoral joint, fibrosis of the talocrural joint, fibrosis of the temporomandibular joint, fibrosis of the metacarpophalangeal joint, fibrosis of the metatarsophalangeal joint, fibrosis of the peri-articular musculature, cellulite and interstitial lung disease.

In some preferred embodiments, the fibrotic disease is arthrofibrosis or a stiffened fibrotic joint.

In some embodiments, recombinant relaxin or a variant or analogue described herein is used to treat an organ or location on the body of a subject, a disease or indication in a subject and or using an administration route as described in Table 1 and/or Table 2.

TABLE 1

| | | |
|---|---|---|
| Administration | Sites (non-joints) | Lung, kidney, liver, heart skin, eye; tendons, |
| routes and | | osteotendinous junctions, tendon-bone interfaces, |
| targets | | entheses, or muscle-tendon insertions, mentioning a slew |
| | | of tendons throughout the body |
| | Sites (joints) | Jaw, spine, shoulder, elbow, wrist, hand, finger, hip, |
| | | knee, ankle, foot, toe; or any other synovial or non- |
| | | synovial joint |
| | Routes of | JOINT INJECTIONS (JI): Intraarticular, periarticular, |
| | administration | intracapsular, pericapsular |
| | | NON-JOINT DENSE CONNECTIVE TISSUE |
| | | INJECTIONS (NJDCTI): intraligamentous, |
| | | periligamentous, intratendinous, peritendinous, |
| | | intraosteotendinous, or periosteotendinous; |
| | | intramusculotendinous, perimusculotendinous, |
| | | perimuscularly, OTHER, NON-ORTHOPEDIC: |
| | | intravenously, intramuscularly, subcutaneously, |
| | | intradermally, intranasally, orally, transcutaneously |
| | | (ionto/electrophoresis), mucosally, gel, cream, ointment, |
| | | lotion, drop, suppository, spray, liquid, powder, |
| | | pulmonary inhalation, ocular. |
| Indications | When to | During or just after a medical procedure; for patients with |
| | administer | stiffened joint or at risk for stiffened joint (treatment or |
| | treatment | prophylactic) |

TABLE 2

| | |
|---|---|
| General causes of fibrosis | Idiopathic, injury (trauma, medical procedure e.g. surgery), immobility |
| | for whatever reason, inflammation, or disease/medical condition |
| Diseases/conditions: | adhesive capsulitis (injury, idiopathic, post-surgical, post-implant) |
| joints (admin via joint | |
| injection) | |
| Diseases/conditions: lung | idiopathic pulmonary fibrosis, cystic fibrosis, hypertension |
| (admin via inhalation) | |
| Diseases/conditions: | hepatitis B or C, long-term alcohol abuse, non-alcoholic steatohepatitis, |
| liver | non-alcoholic fatty liver disease, Cholestasis, autoimmune hepatitis |
| | cirrhosis |
| Diseases/conditions: | chronic kidney disease, end-stage renal disease, renal interstitial fibrosis |
| kidney | |
| Diseases/conditions: heart | heart failure, myocardial infarction, aortic stenosis, hypertrophic |
| | cardiomyopathy |
| Diseases/conditions: | Crohn’s disease, inflammatory bowel disease, enteropathies |
| intestine | |
| Diseases/conditions: skin | scleroderma, keloids, hypertrophic scars, cellulite |
| (admin via intradermal | |
| injection, or transdermal) | |
| Diseases/conditions: | Peyronie’s disease, uterine fibroids, urethral strictures |
| urogenital/gynecological | |
| Diseases/conditions: | Congenital Fibrosis of the Extraocular Muscles, subretinal fibrosis, |
| ocular | epiretinal fibrosis, corneal fibrosis |
| Diseases/conditions: | Dupuytren’s disease, capsular contracture of breast, Plantar |
| connective tissue, fascia | fibromatosis, |
| Diseases/conditions: | Duchenne, Becker, congenital, and other muscular dystrophies, SMA, |
| neuromuscular (admin via | Charcot-Marie-Tooth, arthrogryposis, ALS, club foot, post-polio, CP. |
| joint or peri-joint | |
| injection) | |

In some embodiments, a method is provided in which the method involves identifying a subject diagnosed with one or more diseases selected from the group of diseases listed in Table 1 or Table 2 and administering a formulation of the invention to the subject.

### Methods for Treating a Stiffened Joint

Also provided herein is a method for treating or preventing a stiffened joint in a subject in need thereof with the recombinantly produced relaxin, e.g., relaxin-2 or a variant or analogue described herein. Generally, the method comprises administering to the subject an effective amount of relaxin, e.g., relaxin-2 or a variant or analogue described herein such that the stiffened joint or surrounding tissue area in the subject is treated.

The current methods for treating a stiffened joint include physical therapy or surgical procedures, such as manipulations and releases, which do not offer reliable or consistent results (Diercks R. L. et al., J. Shoulder Elbow Surg. 2004, 13(5):499-502). Physical therapy involves prolonged manipulation by a physical therapist and surgical procedures involves invasive surgical release by a surgeon, followed again by prolonged therapy.

The methods of treatment described herein are advantageous as compared to the currently available methods because they can be used to reliably and effectively treat a stiffened joint or tissue area, while also using a minimally invasive procedure, e.g., an intraarticular injection, which may be performed in an outpatient setting or an office. Thus, the methods of treatment described herein constitute a paradigm shift in the management of a stiffened joint, e.g., a shoulder joint, that may result from fibrosis. The methods of treatment described herein involve minimally invasive procedures, e.g., an intraarticular injection of relaxin-2, e.g., relaxin-2 encapsulated in a sustained release formulation. The intraarticular injection may be repeated as needed until the stiffened joint is successfully treated, e.g., until motion in the joint is restored and pain during motion is eliminated. Successful treatment of a stiffened joint when using the methods of treatment described herein may be accomplished significantly faster and more effectively than when using the currently available methods.

The term “stiffened joint” refers to a joint that may be characterized by a loss of motion, loss of a range of motion or pain during movement. A stiffened joint may be caused by a disease or a medical condition, such as osteoarthritis or inflammation of the joint. A stiffened joint may alternatively be caused by an injury to the joint. A stiffened joint may also result from a medical procedure, e.g., an operation, or from a prolonged immobility of the joint. The term “stiffened joint” includes any joint in a subject, e.g., a human subject, and may include, without limitation, a shoulder joint, an elbow joint, a finger joint, a hip joint, a knee joint or an ankle joint. In a specific embodiment, the stiffened joint is a shoulder joint. The term “stiffened joint” may also be referred herein as “arthrofibrosis”, “capsular fibrosis”, or “fibrosis associated with capsular contracture”.

Pathology of a stiffened joint, e.g., a shoulder joint, includes a thickened glenohumeral joint capsule with adhesions obliterating the axillary fold. The fibrotic capsule adheres to itself and the anatomic neck of the humerus, intraarticular volume is diminished, and synovial fluid in the joint is significantly decreased. Biopsy of the capsule shows a chronic inflammatory infiltrate, with the presence of fibroblasts and transforming myofibroblasts, along with type-I and type-III collagen. Gene and protein expression assays have found components related to fibrosis, inflammation, and chondrogenesis, including increased COL1A1 and COL1A3, interleukin-6 (IL-6), platelet-derived growth factor (PDGF), fibroblast growth factors (FGF) and TMPs, as well as decreased MMP activity. This evidence points to inflammatory changes initiating the recruitment of fibroblasts and immune cells, precipitating the fibrotic process and inappropriate deposition of excess collagen. Alternatively, it is also possible that fibrosis occurs first, followed by inflammation; fibrosis being secondary to defective cell-signaling pathways governing collagen remodeling.

Without wishing to be bound by a specific theory, it is believed that the recombinantly produced relaxin-2 or its analog, when delivered to a joint, e.g., via a solution, hydrogel or particle, intraarticular injection, sustained release formulation, promotes collagen degradation, thereby altering the homeostasis of the extracellular matrix (ECM) in the synovium. This administration results in decreased joint stiffness and increased range of motion of the joint.

The methods of treatment described herein comprise administering relaxin, e.g., relaxin-2 or a variant or analogue described herein to a subject in need thereof.

In some embodiments, methods of treatment described herein result in a treatment of the stiffened joint, such that pain on movement of the joint is reduced, e.g., by at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or more, and is preferably down to a level accepted within the range of normal for an individual who is not affected by a stiffened joint.

In some embodiments, methods of treatment described herein result in restoration of the movement, or a range of the movement, of a joint affected by joint stiffness. For example, treatment of the stiffened joint according to the methods of the invention may result in restoration of the movement, or a range of movement, of a joint affected by joint stiffness, to levels that are at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or 100% of the levels accepted within the range of normal for an individual not affected by a stiffened joint.

In some embodiments, prevention or treatment of a stiffened joint in a subject provided by the methods of treatment described herein is accomplished without significant adverse events, without significant damage to collagenous structures or tissues in the subject, e.g., collagenous structures or tissues of the joint, such as articular cartilage of the joint. For example, methods of treatment described herein provide prevention and treatment of stiffened joint that do not disrupt architecture of the joint. Damage to collagenous structures in the body, e.g., collagenous structures of a joint, may be assessed by methods known in the art, e.g., by measuring levels of various markers in the synovial fluid, such as Cartilage Oligomeric Matrix Protein (COMP), aggrecans, collagen II, proteoglycans, MMPs and inflammatory mediators and cytokines. Imaging techniques such as MRI can also be used to visualize the joint and the cartilage architecture.

### Compositions

For administering to a subject, the recombinantly produced relaxin, e.g., relaxin-2 or a variant or analogue described herein can be comprised in composition, e.g., a pharmaceutical composition. Without limitations, the relaxin, e.g., relaxin-2 or a variant or analogue described herein can be formulated for intravenous, intramuscular, subcutaneous, intradermal, intranasal, oral, transcutaneous, mucosal or intraarticular administration to a subject.

In some embodiments, the relaxin, e.g., relaxin-2 or a variant or analogue described herein can be formulated as a gel, a cream, an ointment, a lotion, a drop, a suppository, a spray, a liquid or a powder composition.

As used herein, the term “pharmaceutical composition” can include any material or substance that, when combined with an active ingredient (e.g., an antifibrotic agent, such as relaxin), allows the ingredient to retain biological activity and is non-reactive with the subject's immune system. Examples include, but are not limited to, any of the standard pharmaceutical carriers such as a phosphate buffered saline solution, emulsions such as oil/water emulsion, and various types of wetting agents. The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material, involved in carrying or transporting the subject agents from one organ, or portion of the body, to another organ, or portion of the body. The term “pharmaceutically acceptable carrier” excludes tissue culture media. Each carrier must be “acceptable” in the sense of being compatible with the other ingredients of the formulation, for example the carrier does not decrease the impact of the agent on the treatment. In other words, a carrier is pharmaceutically inert. The terms “physiologically tolerable carriers” and “biocompatible delivery vehicles” are used interchangeably. Non-limiting examples of pharmaceutical carriers include particle or polymer-based vehicles such as nanoparticles, microparticles, polymer microspheres, or polymer-drug conjugates.

In some embodiments, the pharmaceutical composition is a liquid dosage form or solid dosage form. Liquid dosage forms for oral administration include, but are not limited to, pharmaceutically acceptable emulsions, microemulsions, solutions, suspensions, syrups and elixirs. In addition, the liquid dosage forms can contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

In some embodiments, the liquid dosage form is prepared at or near the point of care by reconstituting or resuspending a provided lyophilisate or lyophilized powder of a formulation disclosed herein using a diluent solution.

Solid dosage forms for oral administration include capsules, tablets, pills, powders, and granules. In such solid dosage forms, the agents described herein are mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form can also comprise buffering agents.

Solid compositions of a similar type can also be employed as fillers in soft and hard-filled gelatin capsules using such excipients as lactose or milk sugar as well as high molecular weight polyethylene glycols, and the like. The solid dosage forms of tablets, dragées, capsules, pills, and granules can be prepared with coatings and shells such as enteric coatings and other coatings well known in the pharmaceutical formulating art. They can optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type can also be employed as fillers in soft and hard-filled gelatin capsules using such excipients as lactose or milk sugar as well as high molecular weight polyethylene glycols, and the like.

In some embodiments, the solid dosage form is a lyophilized powder. In some dosage forms, the lyophilized powder solid dosage form is intended to be resuspended or reconstituted with a diluent.

The recombinant relaxin or a variant or analogue thereof can also be in micro-encapsulated form with one or more excipients as noted above. The solid dosage forms of tablets, dragées, capsules, pills, and granules can be prepared with coatings and shells such as enteric coatings, release controlling coatings and other coatings well known in the pharmaceutical formulating art. In such solid dosage forms, the agent can be admixed with at least one inert diluent such as sucrose, lactose and starch. Such dosage forms can also comprise, as in normal practice, additional substances other than inert diluents, e.g., tableting lubricants and other tableting aids such as magnesium stearate and microcrystalline cellulose. In the case of capsules, tablets and pills, the dosage forms can also comprise buffering agents. They can optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes.

Pharmaceutical compositions include formulations suitable for oral administration may be provided as discrete units, such as tablets, capsules, cachets, syrups, elixirs, prepared food items, microemulsions, solutions, suspensions, lozenges, or gel-coated ampules, each containing a predetermined amount of the active compound; as powders or granules; as solutions or suspensions in aqueous or non-aqueous liquids; or as oil-in-water or water-in-oil emulsions.

Accordingly, formulations suitable for rectal administration include gels, creams, lotions, aqueous or oily suspensions, dispersible powders or granules, emulsions, dissolvable solid materials, douches, and the like can be used. The formulations are preferably provided as unit-dose suppositories comprising the active ingredient in one or more solid carriers forming the suppository base, for example, cocoa butter. Suitable carriers for such formulations include petroleum jelly, lanolin, polyethyleneglycols, alcohols, and combinations thereof. Alternatively, colonic washes with the rapid recolonization deployment agent of the present invention can be formulated for colonic or rectal administration.

In some embodiments, the recombinant relaxin or a variant or analogue thereof can be formulated in a sustained release formulation. The sustained release formulations of the invention consist of a hydrogel, microparticle or some matrix encapsulation of the recombinant relaxin or a variant or analogue thereof. In some embodiments, the sustained release formulation comprises the relaxin or a variant or analogue thereof encapsulated by or chemically bound to the depot support material via a linker. The linker may, comprise a polymer, a non-cleavable linker, or a cleavable linker, either through chemical or enzymatic means. The depot may be formed in situ following mixing of the recombinant relaxin or a variant or analogue thereof with the material. The depot may be formed prior to mixing of the recombinant relaxin or a variant or analogue thereof with the material.

The sustained release formulation comprising the recombinant relaxin or a variant or analogue thereof may be in the form of a hydrogel or microparticle which comprises one or more polymers. Without limitations, the polymer can be an aliphatic polyester or a vinyl polymer.

As used herein the term “aliphatic polyester” refers to the following without limitation, poly(lactide), poly(glycolide), poly(lactide-co-glycolide), poly(γ-valerolactone), polyethylene glycol (PEG), alginate, agarose, poly(hydroxyvalerate), poly(hydroxybutyrate), poly(3-hydroxybutyrate-co-4-hydroxybutyrate), poly(hydroxyhexanoate), poly(butylene succinate), poly(alkylene alkanoate), poly(propylene succinate), poly(ethylene succinate), poly(ε-caprolactone), poly(ethylene glycol dimethacrylate), gelatin, collagen, agarose, pectin, poly(lysine), bolaamphiphiles, glycosyl-nucleosides, and fluorocarbon chains. It is noted that an aliphatic polyester for use in a formulation described herein can be of molecular weight 10,000-200,000 daltons; 10,000-150,000 daltons; or 25,000-125,000 daltons; or 40,00-100,000 daltons; 10,000-30,000 daltons; 30,000-50,000 daltons; 50,000-70,000 daltons; 70,000-90,000 daltons; 90,000-120,000 daltons; or 120,000-150,000 daltons.

As used herein the term “vinyl polymer” refers to molecules including without limitation, poly(vinyl alcohols) poly(vinyl chlorides), poly(ethylene), poly(propylene), poly(styrene), poly(styrene sulfonate), poly(vinyl chloride), poly(vinyl alcohol), poly(vinyl acetate), poly(vinyl cyanide), poly(vinyl fluoride), poly(vinyl nitrate), poly(vinyl toluene), poly(vinylpyrrolidone), poly(vinylpolypyrrolidone), pluronic polyol, polyoxamer, poly(uronic acid), poly(anhydride), polyNIPAM, poly(acrylates, poly(acrylamides), poly(betaines), tween (20, 40, 60, 80), decyl glucoside glycerol monostearate, glycerol monolaurate, sorbitan monolaurate, sorbitan monostearate, triton x-100, carboxylmethylcellulose, hypromellose, and pluronic F-127. In some embodiments, the listed molecules may be utilized for their emulsification and stabilization properties.

Some preferred polymers for use in a sustained release relaxin formulation include, but are not limited to, poly-lactide-co-glycolide, polycaprolactone, poly-epsilon-caprolactone, polyethylene glycol (PEG), alginate, agarose, poly(ethylene glycol dimethacrylate), polylactic acid, polyglycolic acid, gelatin, collagen, agarose, pectin, poly(lysine), polyhydroxybutyrate, polyphosphazines, poly(vinyl alcohol), poly(alkylene oxide), poly(ethylene oxide), poly(allylamine), poly(acrylate), poly(4-aminomethylstyrene), pluronic polyol, polyoxamer, poly(uronic acid), poly(anhydride), poly(vinylpyrrolidone), bolaamphiphiles, glycosyl-nucleosides, and fluorocarbon chains.

In some embodiments of any one of the aspects described herein, the sustained release formulation comprises poly-lactide-co-glycolide. For example, the sustained release formulation comprises poly-lactide-co-glycolide with a molar ratio of 15:85-25:75, lactide:glycolide; poly-lactide-co-glycolide with a molar ratio of 25:75-35:65, lactide:glycolide; poly-lactide-co-glycolide with a molar ratio of 35:65-45:55, lactide:glycolide; poly-lactide-co-glycolide with a molar ratio of 45:55-55:45, lactide:glycolide; poly-lactide-co-glycolide with a molar ratio of 55:45-65:35, lactide:glycolide; poly-lactide-co-glycolide with a molar ratio of 65:35-75:25, lactide:glycolide; poly-lactide-co-glycolide with a molar ratio of 75:25-85:15, lactide:glycolide; poly-lactide-co-glycolide with a molar ratio of about 50:50, lactide:glycolide; poly-lactide-co-glycolide with a molar ratio of about 45:55, lactide:glycolide; poly-lactide-co-glycolide with a molar ratio of about 55:45, lactide:glycolide; poly-lactide-co-glycolide with a molar ratio of about 40:60, lactide:glycolide; or poly-lactide-co-glycolide with a molar ratio of about 60:40, lactide:glycolide.

In some embodiments of any one of the aspects described herein, the sustained release formulation comprises a vinyl polymer. For example, the sustained release formulation comprises a vinyl polymer that is of molecular weight 10,000-200,000 Daltons; 10,000-150,000 Daltons; or 25,000-125,000 Daltons; or 40,00-100,000 Daltons; 10,000-30,000 Daltons; 30,000-50,000 Daltons; 50,000-70,000 Daltons; 70,000-90,000 Daltons; 90,000-120,000 Daltons; or 120,000-150,000 Daltons.

The amount of the vinyl polymer in the formulation can be about 0.01-0.1% of total mass; 0.1-0.3% of total mass; 0.2-0.9% of total mass; 0.3-0.7% of total mass; 0.4-0.6% of total mass; 0.3-0.6% of total mass; 0.6-1.0% of total mass; 1.0-5.0% of total mass; 5.0-10.0% of total mass; 10.0-30.0% of total mass; 0.1% of total mass; 0.2% of total mass; 0.3% of total mass; 0.4% of total mass; 0.5% of total mass; 0.6% of total mass; 0.7% of total mass; 0.8% of total mass; 0.9% of total mass; 10% of total mass; 15% of total mass; 20% of total mass; 25% of total mass; 30% of total mass; or 33% of total mass.

In some embodiments of any one of the aspects, the vinyl polymer is poly(vinyl alcohol). In some other embodiments of any one of the aspects, the vinyl polymer is poly(pyrrolidone).

In some embodiments of any of the aspects described herein, the recombinant relaxin or a variant or analogue thereof is administered to a subject by controlled- or delayed-release means. Ideally, the use of an optimally designed controlled-release preparation in medical treatment is characterized by a minimum of drug substance being employed to cure or control the condition in a minimum amount of time. Advantages of controlled-release formulations include: 1) extended activity of the drug; 2) reduced dosage frequency; 3) increased patient compliance; 4) usage of less total drug; 5) reduction in local or systemic side effects; 6) minimization of drug accumulation; 7) reduction in blood level fluctuations; 8) improvement in efficacy of treatment; 9) reduction of potentiation or loss of drug activity; and 10) improvement in speed of control of diseases or conditions. (Kim, Cherng-ju, Controlled Release Dosage Form Design, 2 (Technomic Publishing, Lancaster, Pa.: 2000)). Controlled-release formulations can be used to control a compound of formula (I)'s onset of action, duration of action, plasma levels within the therapeutic window, and peak blood levels. In particular, controlled- or extended-release dosage forms or formulations can be used to ensure that the maximum effectiveness of an agent is achieved while minimizing potential adverse effects and safety concerns, which can occur both from under-dosing a drug (i.e., going below the minimum therapeutic levels) as well as exceeding the toxicity level for the drug.

A variety of known controlled- or extended-release dosage forms, formulations, and devices can be adapted for use with any agent described herein. Examples include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; 5,733,566; and 6,365,185, each of which is incorporated herein by reference in their entireties. These dosage forms can be used to provide slow or controlled-release of one or more active ingredients using, for example, hydroxypropylmethyl cellulose, other polymer matrices, gels, permeable membranes, osmotic systems (such as OROS® (Alza Corporation, Mountain View, Calif. USA)), multilayer coatings, microparticles, liposomes, or microspheres or a combination thereof to provide the desired release profile in varying proportions. Additionally, ion exchange materials can be used to prepare immobilized, adsorbed salt forms of the disclosed compounds and thus effect controlled delivery of the drug. Examples of specific anion exchangers include, but are not limited to, DUOLITE® A568 and DUOLITE® AP143 (Rohm&Haas, Spring House, Pa. USA).

In some embodiments, any aforementioned polymers, prior to or after loading of the recombinant relaxin or a variant or analogue thereof, may be characterized (e.g. size, molecular weight, charge, secondary structure, and purity) by techniques including, but not limited to, gel permeation chromatography, high performance liquid chromatography, ultra-performance liquid chromatography, MALDI-TOF mass spectroscopy, viscometry, and light scattering (e.g. multi-angle, low angle laser).

In some embodiments, the rate of release of recombinant relaxin or a variant or analogue thereof may be characterized by techniques including, but not limited to, high performance liquid chromatography, ultra-performance liquid chromatography, fast protein liquid chromatography, enzyme linked immunosorbent assay, and ligand binding assay. In some embodiments, the release rate of the recombinant relaxin or a variant or analogue thereof is measured as the concentration of the recombinant relaxin or a variant or analogue thereof in any biologically relevant liquid solution or suspension or medium (e.g. saline, mammalian cell culture media, synthetic synovial fluid, synovial fluid, serum, synthetic serum, plasma, synthetic plasma and deionized water) that the formulation is also in. In specific embodiments, the formulation and biologically relevant liquid solution or suspension is maintained at a specific temperature. In specific embodiments, the formulation and biologically relevant liquid solution or suspension is agitated or mixed at a set or varying rate of motion. In specific embodiments, the concentration of relaxin released into the biologically relevant liquid solution or suspension is measured using a direct enzyme linked immunosorbent assay. In specific embodiments, the concentration of the recombinant relaxin or a variant or analogue thereof released into the biologically relevant liquid solution or suspension is measured using an indirect enzyme linked immunosorbent assay. In specific embodiments, the concentration of the recombinant relaxin or a variant or analogue thereof released into the biologically relevant liquid solution or suspension is measured using a sandwich enzyme linked immunosorbent assay. In a preferred embodiment, the concentration of the recombinant relaxin or a variant or analogue thereof released into the biologically relevant liquid solution or suspension is measured using the Human Relaxin-2 Quantikine ELISA Kit from Bio-techne corporation.

In some embodiments, the size and morphology (e.g. diameter, sphericity, and porosity) of relaxin microparticles may be characterized by techniques including, but not limited to, dynamic light scattering, coulter counter, microscopy, sieve analysis, dynamic image analysis, static image analysis, and laser diffraction.

In some embodiments, the total loaded content of recombinant relaxin in relaxin microparticles (e.g. percent of recombinant relaxin as weight/volume, percent of recombinant relaxin as weight/weight) may be characterized by techniques including, but not limited to, mass balance, limited to, high performance liquid chromatography, ultra-performance liquid chromatography, fast protein liquid chromatography, enzyme linked immunosorbent assay, and ligand binding assay. In some embodiments, the formulation may be purified and dissolved to assess total loaded content of relaxin.

In some embodiments, the total loaded content (i.e. mass) of recombinant relaxin in relaxin microparticles is measured as the concentration of relaxin in any liquid solution, suspension or medium (e.g. saline, mammalian cell culture media, synthetic synovial fluid, synovial fluid, serum, synthetic serum, plasma, synthetic plasma, methylene chloride, acetonitrile, ethyl acetate, and deionized water) that the total formulation may be dissolved in. In specific embodiments, the concentration of relaxin after formulation dissolution in the liquid solution, suspension, or medium is measured using a direct enzyme linked immunosorbent assay. In specific embodiments, the concentration of relaxin after formulation dissolution in the liquid solution, suspension, of medium is measured using an indirect enzyme linked immunosorbent assay. In specific embodiments, the concentration of relaxin after formulation dissolution in the liquid solution, suspension, of medium is measured using a sandwich enzyme linked immunosorbent assay. In a preferred embodiment, the concentration of relaxin after formulation dissolution in the liquid solution, suspension, of medium is measured using the Human Relaxin-2 Quantikine ELISA Kit from Bio-techne corporation.

In certain embodiments, the sustained release formulation comprises PEG, e.g., a linear PEG or a branched PEG. In certain embodiments, the molecular weight of the PEG is more than 0.2 kDa, more than 0.5 kDa, more than 1 kDa, more than 5 kDa, more than 10 kDa, or more than 20 kDa

In some embodiments, the hydrogel comprises PEG-based crosslinkers with an internal thioester that will be reacted with dendrons to prepare hydrogels. These hydrogels may be prepared in varying weight percent to modulate mechanical properties. In specific embodiments the internal thioester allows for controlled dissolution through the use of a cysteine methyl ester solution. In specific embodiments, the gels material properties including, but not limited to, release profile, young's modulus, sheer modulus, hydrophobicity, and, elasticity can be varied through modification of the thioester moiety to modulate material properties of hydrogel.

In some embodiments of any one of the aspects, the aliphatic polyester is terminated by an ester functional group.

In some embodiments of any one of the aspects, the aliphatic polyester is terminated by an alkyl-ester functional group.

In some embodiments of any one of the aspects, the aliphatic polyester is terminated by a carboxylic acid functional group.

In some embodiments of any one of the aspects, the aliphatic polyester is terminated by a that allows for bioconjugation between the aliphatic polymer and a biomolecule, e.g., recombinant relaxin or a variant or analogue thereof. For example, the aliphatic polyester is terminated by an amine functional group, an isocyanate functional group, an isothiocyanate functional group, a benzoyl fluoride functional group, a maleimide functional group, an iodoacetamide functional group, a 2-thiopyridine functional groups, a 3-arylpropiolonitrile functional group, a diazonium salt, an aldehyde, a ketone, an azide, an alkyne, a cyclooctyne, or a phosphine.

In some embodiments of any one of the aspects described herein, the sustained release formulation is in form of microparticles. The diameter of the microparticles can be 1-100 μm. In some embodiments of any one of the aspects described herein, the diameter of the microparticles is 1-75 μm; or 1-50 μm; or 5-50 μm; or 25-50 μm; or 30-50 μm; or 40-50 μm; or 5-10 μm; 5-8 μm; 8-12 μm; 12-18 μm; 18-25 μm; 25-35 μm; 35-45 μm; 45-50 μm; 1 μm; 2 μm; 3 μm; 4 μm; 5 μm; 6 μm; 7 μm; 8 μm; 9 μm; 10 μm; 15 μm; 20 μm; 25 μm; 30 μm; 35 μm; 40 μm; 45 μm; 50 μm; 75 μm; 100 μm; 150 μm; or 200 μm.

In some embodiments of any one of the aspects described herein, the recombinant relaxin or a variant or analogue thereof is 0.005-5% of the total formulation mass. In some embodiments of any one of the aspects described herein, the recombinant relaxin or a variant or analogue thereof is 0.01-10%, 0.01-33%, or 0.1-5% of the total formulation mass; or 0.2-4% of the total formulation mass; or 0.3-3% of the total formulation mass; or 0.5-2% of the total formulation mass; or 0.5-1.5% of the total formulation mass; or 0.5-3% of the total formulation mass; or 1-2% of the total formulation mass; or 1-5% of the total formulation mass; or 3-7% of the total formulation mass; or 5-10% of the total formulation mass.

In some embodiments of any one of the aspects described herein, the recombinant relaxin or a variant or analogue thereof is about 0.005-0.01% of the total formulation mass; 0.01-0.05% of the total formulation mass; 0.05-0.1% of the total formulation mass; 0.1-0.5% of the total formulation mass; 0.5-1.0% of the total formulation mass; 1.0-2.5% of the total formulation mass; 2.5-5.0% of the total formulation mass; 0.25% of the total formulation mass; 0.5% of the total formulation mass; 0.75% of the total formulation mass; 1% of the total formulation mass; 1.25% of the total formulation mass; 1.5% of the total formulation mass; 1.75% of the total formulation mass; 2% of the total formulation mass; 2.5% of the total formulation mass; 3% of the total formulation mass; or 5% of the total formulation mass.

In some embodiments of any one of the aspects, the formulation comprises microparticles comprising recombinant relaxin or a variant or analogue thereof in an amount of about 1% by weight of the microparticlee and PLGA having a lactide:glycolide of about 50:50.

In some embodiments of any one of the aspects, the formulation comprises microparticles comprising recombinant relaxin or a variant or analogue thereof in an amount of about 1% by weight of the microparticles, PLGA having a lactide:glycolide of about 50:50, and PVA at a concentration of about 0.5% by weight.

In some embodiments of any one of the aspects, the formulation comprises microparticles comprising recombinant relaxin or a variant or analogue thereof in an amount of about 1% by weight of the microparticles, PLGA having a lactide:glycolide of about 60:40, and PVA at a concentration of about 0.5% by weight.

In some embodiments of any one of the aspects, the formulation comprises microparticles comprising recombinant relaxin or a variant or analogue thereof in an amount of about 1% by weight of the microparticles, PLGA having a lactide:glycolide of about 40:60, and PVA at a concentration of about 0.5% by weight.

It is noted that the microparticles can be in dried form, e.g., a powder, or the microparticles can be suspended in a liquid solution. For example, the microparticles can be suspended in a sodium chloride liquid solution or a sodium carboxymethylcellulose solution.

Accordingly, inn some embodiments of any one of the aspects, the formulation comprises microparticles suspended in a sodium chloride liquid solution. The amount of the sodium chloride is 0.5-1.5 w/w %; or between 0.75-1.25 w/w %; or about 0.5 w/w %; or about 0.6 w/w %; or about 0.7 w/w %; or about 0.8 w/w %; or about 0.9 w/w %; or about 1.0 w/w %; or about 1.1 w/w %; or about 1.2 w/w %; or about 1.3 w/w %; or about 1.4 w/w %; or about 1.5 w/w % of the liquid solution.

In some embodiments of any one of the aspects, the formulation comprises microparticles suspended in a sodium carboxymethylcellulose solution. The amount of the sodium carboxymethylcellulose solution can be 0.1-1.0 w/w %; or between 0.25-.75 w/w %; or about 0.1 w/w %; or about 0.2 w/w %; or about 0.3 w/w %; or about 0.4 w/w %; or about 0.5 w/w %; or about 0.6 w/w %; or about 0.7 w/w %; or about 0.8 w/w %; or about 0.9 w/w %; or about 1.0 w/w % of the liquid solution.

For the recombinant relaxin or a variant or analogue thereof to have a sustained clinical antifibrotic effect, it is physiologically desirable for the temporal concentration of recombinant relaxin or a variant or analogue thereof to be above the minimum effective concentration for a sustained duration. A bolus dose of relaxin is reported to not be effective in animals. A sustained dose of relaxin is reported to be effective in animals. A constant sustained dose of recombinant relaxin or a variant or analogue thereof may be achieved by the release of recombinant relaxin or a variant or analogue thereof from a microparticle with a linear rate of release (i.e. one having no bolus effect or burst-release effect).

Accordingly, in some embodiments of any one of the aspects, the formulation is a sustained release formulation. In some embodiments of any one of the aspects, the recombinant relaxin or a variant or analogue thereof is released from the sustained release formulation over an extended period of time. For example, the recombinant relaxin or a variant or analogue thereof is released from the sustained release formulation over a period of least 1 day; or at least 2 days; or at least 3 days; or at least 4 days; or at least 5 days; or at least 6 days; or at least 1 week; or at least 2 weeks; or at least 3 weeks; or at least 4 weeks; or at least 5 weeks, or at least 6 weeks; or at least 8 weeks; or at least 9 weeks; at least 10 weeks; or at least 12 weeks; or at least 15 weeks; or between 1-5 days; or between 2-5 days; or between 1-2 days; or between 2-3 days; or between 3-4 days; or between 4-5 days; or between 3-10 days; or between 1-15 weeks; or between 2-10 weeks; or between 4-8 weeks; or between 8-15 weeks; or about 1 day; or about 2 days; or about 3 days; or about 4 days; or about 5 days; or about 6 days; or about 1 week; or about 2 weeks; or about 3 weeks; or about 4 weeks; or about 5 weeks; or about 6 weeks; or about 7 weeks; or about 8 weeks; or about 9 weeks; or about 10 weeks, or more.

Some exemplary formulations that are useful for formulating the recombinant relaxin or variant or analogue thereof of the invention are described in U.S. patent application Ser. No. 16/339,659 and Ser. No. 17/327,011, contents of which are incorporated herein by reference in their entirety.

### Dosage

“Unit dosage form” as the term is used herein refers to a dosage for suitable one administration. By way of example a unit dosage form can be an amount of therapeutic disposed in a delivery device, e.g., a syringe or intravenous drip bag. In one embodiment of any of the aspects, a unit dosage form is administered in a single administration. In another, embodiment more than one-unit dosage form can be administered simultaneously.

The dosage of the agent as described herein can be determined by a physician and adjusted, as necessary, to suit observed effects of the treatment. With respect to duration and frequency of treatment, it is typical for skilled clinicians to monitor subjects in order to determine when the treatment is providing therapeutic benefit, and to determine whether to administer further cells, discontinue treatment, resume treatment, or make other alterations to the treatment regimen. The dosage should not be so large as to cause adverse side effects, such as cytokine release syndrome. Generally, the dosage will vary with the age, condition, and sex of the patient and can be determined by one of skill in the art. The dosage can also be adjusted by the individual physician in the event of any complication.

The effective dose can be estimated initially from cell culture assays. A dose can be formulated in animals. Generally, the compositions are administered so that the recombinant relaxin or a variant or analogue thereof is used or given at a dose from 1 mg/kg to 1000 mg/kg; 1 mg/kg to 500 mg/kg; 1 mg/kg to 150 mg/kg, 1 mg/kg to 100 mg/kg, 1 mg/kg to 50 mg/kg, 1 mg/kg to 20 mg/kg, 1 mg/kg to 10 mg/kg, 1 μg/kg to 1 mg/kg, 100 mg/kg to 100 mg/kg, 100 mg/kg to 50 mg/kg, 100 mg/kg to 20 mg/kg, 100 mg/kg to 10 mg/kg, 100 μg/kg to 1 mg/kg, 1 mg/kg to 100 mg/kg, 1 mg/kg to 50 mg/kg, 1 mg/kg to 20 mg/kg, 1 mg/kg to 10 mg/kg, 10 mg/kg to 100 mg/kg, 10 mg/kg to 50 mg/kg, or 10 mg/kg to 20 mg/kg. It is to be understood that ranges given here include all intermediate ranges, for example, the range 1 mg/kg to 10 mg/kg includes 1 mg/kg to 2 mg/kg, 1 mg/kg to 3 mg/kg, 1 mg/kg to 4 mg/kg, 1 mg/kg to 5 mg/kg, 1 mg/kg to 6 mg/kg, 1 mg/kg to 7 mg/kg, 1 mg/kg to 8 mg/kg, 1 mg/kg to 9 mg/kg, 2 mg/kg to 10 mg/kg, 3 mg/kg to 10 mg/kg, 4 mg/kg to 10 mg/kg, 5 mg/kg to 10 mg/kg, 6 mg/kg to 10 mg/kg, 7 mg/kg to 10 mg/kg, 8 mg/kg to 10 mg/kg, 9 mg/kg to 10 mg/kg, and the like. Further contemplated is a dose (either as a bolus or continuous infusion) of about 0.1 mg/kg to about 10 mg/kg, about 0.3 mg/kg to about 5 mg/kg, or 0.5 mg/kg to about 3 mg/kg. It is to be further understood that the ranges intermediate to those given above are also within the scope of this invention, for example, in the range 1 mg/kg to 10 mg/kg, for example use or dose ranges such as 2 mg/kg to 8 mg/kg, 3 mg/kg to 7 mg/kg, 4 mg/kg to 6 mg/kg, and the like.

### Parenteral Dosage Forms

Parenteral dosage forms of a recombinant relaxin or a variant or analogue thereof described herein can be administered to a subject by various routes, including, but not limited to, subcutaneous, intravenous (including bolus injection), intramuscular, and intraarterial. Since administration of parenteral dosage forms typically bypasses the patient's natural defenses against contaminants, parenteral dosage forms are preferably sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, controlled-release parenteral dosage forms, and emulsions.

The phrases “parenteral administration” and “administered parenterally” as used herein means modes of administration other than enteral and topical administration, usually by injection, and includes, without limitation, intravenous, intramuscular, perimuscular, intraarterial, intrathecal, intraventricular, intracapsular, pericapsular, intraorbital, intracardiac, intradermal, peridermal, intraperitoneal, transtracheal, subcutaneous, subcuticular, intraarticular, periarticular, sub capsular, subarachnoid, intraspinal, intracerebro spinal, and intrasternal injection, infusion and other injection or infusion techniques, without limitation. Without limitations, oral administration can be in the form of solutions, suspensions, tablets, pills, capsules, sustained-release formulations, oral rinses, powders and the like. Suitable vehicle solutions that can be used to provide parenteral dosage forms of the invention are well known to those skilled in the art. As used herein, the phrase “vehicle solutions” include, without limitation: sterile water; water for injection USP; saline solution; sodium carboxymethylcellulose; glucose solution; aqueous vehicles such as but not limited to, sodium chloride injection, Ringer's injection, dextrose injection, dextrose and sodium chloride injection, and lactated Ringer's injection; water-miscible vehicles such as, but not limited to, ethyl alcohol, polyethylene glycol, and propylene glycol; and non-aqueous vehicles such as, but not limited to, corn oil, cottonseed oil, peanut oil, sesame oil, ethyl oleate, isopropyl myristate, and benzyl benzoate.

### Efficacy

The efficacy of recombinant relaxin or a variant or analogue thereof described herein, e.g., for the treatment of a disease or disorder associated with fibrosis, can be determined by the skilled practitioner. However, a treatment is considered “effective treatment,” as the term is used herein, if one or more of the signs or symptoms of the fibrotic disease are altered in a beneficial manner, other clinically accepted symptoms are improved, or even ameliorated, or a desired response is induced e.g., by at least 10% following treatment according to the methods described herein. Efficacy can be assessed, for example, by measuring a marker, indicator, symptom, and/or the incidence of a condition treated according to the methods described herein or any other measurable parameter appropriate. Efficacy can also be measured by a failure of an individual to worsen as assessed by hospitalization, or need for medical interventions (i.e., progression of the disease or disorder, as measured by symptoms of the disease or disorder). Methods of measuring these indicators are known to those of skill in the art and/or are described herein.

Efficacy can be assessed in animal models of a condition described herein, for example, a mouse model or an appropriate animal model of a fibrotic disease or disorder, as the case may be. When using an experimental animal model, efficacy of treatment is evidenced when a statistically significant change in a marker is observed.

In some embodiments, efficacy of treatment includes the minimization of foreign-body-response or immune reaction after administration. For example, the administration of a vehicle control formulation (e.g. a PLGA microparticle containing no therapeutic agent) may elicit macrophage and immune activation as well as inflammation, whereas the administration of a formulation described by the present invention may elicit a lower immune response or entirely abrogate the elicited immune response at any point throughout the treatment and assessment after administration.

In some embodiments, foreign body response resulting from administration of a formulation described by the present invention may be reduced or abrogated compared to foreign body response resulting from administration of a PLGA microparticle containing a steroid as the therapeutic agent.

### Routes of Administration

It is noted that the terms “administered” and “subjected” are used interchangeably in the context of treatment of a disease or disorder. In jurisdictions that forbid the patenting of methods that are practiced on the human body, the meaning of “administering” of a composition to a human subject shall be restricted to prescribing a controlled substance that a human subject will be administer to the subject by any technique (e.g., orally, inhalation, topical application, injection, insertion, etc.). The broadest reasonable interpretation that is consistent with laws or regulations defining patentable subject matter is intended. In jurisdictions that do not forbid the patenting of methods that are practiced on the human body, the “administering” of compositions includes both methods practiced on the human body and also the foregoing activities.

As used herein, the term “administer” refers to the placement of a composition into a subject by a method or route which results in at least partial localization of the composition at a desired site such that desired effect is produced. Recombinant relaxin or a formulation comprising the recombinant relaxin, e.g., a recombinant relaxin loaded depot can be administered by any appropriate route known in the art including, but not limited to, oral or parenteral routes, including intravenous, intramuscular, subcutaneous, transdermal, airway (aerosol), pulmonary, nasal, rectal, and topical (including buccal and sublingual) administration.

Exemplary modes of administration include, but are not limited to, injection, infusion, instillation, inhalation, or ingestion. “Injection” includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection and infusion. It is noted that administration can be local or systemic.

In some embodiments of any one of the aspect described herein, said administering is via inhalation as an aerosol, via intra-articular injection, via peri-articular injection, via intramuscular injection, via perimuscular injection, via intradermal injection, via subcutaneous injection, via intracapsular injection, via pericapsular injection, via intraligamentous injection, via periligamentous injection, via intratendinous injection, via peritendinous injection, via intramusculotendionous injection, via perimusculotendinous injection, via intraosteotendinous injection, via periosteotendinous injection. Methods of treatment described herein comprise administering a recombinant relaxin or a variant or analogue thereof to a subject using a depot.

Administering the recombinant relaxin or a variant or analogue thereof loaded depot can be performed by a number of people working in concert and can include, for example, prescribing relaxin or an analog or a variant thereof to be administered to a subject via a depot and/or providing instructions, directly or through another, to take the relaxin or an analog or a variant thereof, either by self-delivery via a depot, e.g., as by oral delivery, subcutaneous delivery, intravenous delivery through a central line, etc., or for delivery by a trained professional, e.g., intra-articular delivery, intravenous delivery, intramuscular delivery, intratumoral delivery, etc.

In a preferred embodiment, the recombinant relaxin or a variant or analogue thereof is administered locally, e.g., directly to or into a joint of a subject using a depot. Local administration of the agent (e.g., relaxin) loaded depot by an intraarticular injection or by topical application to the joint, or in the tissue surrounding the joint is advantageous because it allows delivery of a smaller dose of the agent to the subject and avoids the side-effects associated with systemic delivery, such as back pain and joint pain.

In some embodiments, the recombinant relaxin or a variant or analogue thereof loaded depot is administered to the subject by an intraarticular injection. In one embodiment, the relaxin or its analog loaded depot is administered to the subject via a single intraarticular injections. In some embodiments, the recombinant relaxin or a variant or analogue thereof loaded depot is administered to the subject via multiple intraarticular injections. The multiple intraarticular injections of the recombinant relaxin or a variant or analogue thereof loaded depot may be administered to a subject at regularly spaced time intervals, e.g., every day, every 2 days, every 3 days, every 4 days, every 5 days, every 6 days, every 7 days, every 8 days, every 9 days, every 10 days, every 11 days, every 12 days every 13 days or every 14 days. A course of treatment consisting of multiple intraarticular injections of recombinant relaxin or a variant or analogue thereof loaded depot may be repeated.

The intraarticular injection of the recombinant relaxin or a variant or analogue thereof loaded depot may be accomplished by using a syringe with a needle suited for an intraarticular injection. A needle suitable for an intraarticular injection may be selected from the group consisting of a 30G needle, a 29G needle, a 28G needle, a 27G needle, a 26sG needle, a 26G needle, a 25.5G needle, a 25sG needle, a 25G needle, a 24.5G needle, a 24G needle, a 23.5G needle, a 23sG needle, a 23G needle, a 22.5G needle, a 22sG needle, a 22G needle, a 21.5G needle, a 21G needle, a 20.5G needle, a 20G needle, a 19.5G needle, a 19G needle, a 18.5G needle and an 18G needle. In some preferred embodiments, the recombinant relaxin or a variant or analogue thereof loaded depot is administered via a 21G needle.

In some other preferred embodiments, the recombinant relaxin or a variant or analogue thereof loaded depot may be administered to a subject topically, e.g., transcutaneously. For example, the recombinant relaxin or a variant or analogue thereof loaded depot may be administered as a gel, a cream, an ointment, a lotion, a drop, a suppository, a spray, a liquid or a powder composition that is applied topically to a joint, e.g., a finger joint.

In some embodiments, the recombinant relaxin or a variant or analogue thereof loaded depot may be administered to a subject during a medical procedure, e.g., a surgery, to treat or prevent a stiffened joint. Because stiffened joint may result from a surgery, administering relaxin during surgery may prevent formation of a stiffened joint in a subject. In some embodiments, recombinant relaxin or a variant or analogue thereof loaded depot may be administered through a cannula or an incision.

In some embodiments, the recombinant relaxin or a variant or analogue thereof loaded depot may be administered during an outpatient fluoroscopic or ultrasound guided procedure.

In some preferred embodiments, the recombinant relaxin or a variant or analogue thereof loaded depot is administered to the subject locally in a sustained release formulation. Administering the recombinant relaxin or a variant or analogue thereof as a sustained release formulation is advantageous because it avoids repeated injections and can deliver a therapeutic dose of the relaxin in a consistent and reliable manner, and over a desired period of time. Exemplary sustained release formulations that may be used to delivery polypeptides, are described in Vaishya et al., Expert. Opin. Drug Deliv. 2015, 12(3):415-40, the entire contents of which are incorporated herein by reference.

### Combinational Therapy

In some embodiments of any one of the aspects, the recombinant relaxin or a variant or analogue thereof described herein is used as a monotherapy. In some embodiments of any one of the aspects, the recombinant relaxin or a variant or analogue thereof described herein can be used in combination with other known agents and therapies (i.e. cotherapies) for a disease, condition, or disorder, such as a disease, condition, or disorder associated with fibrosis. Administered “in combination,” as used herein, means that two (or more) different treatments are delivered to the subject during the course of the subject's affliction with the disorder, e.g., the two or more treatments are delivered after the subject has been diagnosed with the disorder (a fibrotic disease or disorder) and before the disorder has been cured or eliminated or treatment has ceased for other reasons.

In some embodiments, the delivery of one treatment is still occurring when the delivery of the second begins, so that there is overlap in terms of administration. This is sometimes referred to herein as “simultaneous” or “concurrent delivery.” In other embodiments, the delivery of one treatment ends before the delivery of the other treatment begins. In some embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In some embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered. The recombinant relaxin or a variant or analogue thereof described herein and the at least one additional therapy can be administered simultaneously, in the same or in separate compositions, or sequentially. For sequential administration, the recombinant relaxin or a variant or analogue thereof described herein can be administered first, and the additional agent can be administered second, or the order of administration can be reversed. The recombinant relaxin or a variant or analogue thereof and/or other therapeutic agents, procedures or modalities can be administered during periods of active disorder, or during a period of remission or less active disease. The recombinant relaxin or a variant or analogue thereof described herein can be administered before another treatment, concurrently with the treatment, post-treatment, or during remission of the disorder.

When administered in combination, the recombinant relaxin or a variant or analogue thereof and the additional agent (e.g., second or third agent), or all, can be administered in an amount or dose that is higher, lower or the same as the amount or dosage of each agent used individually, e.g., as a monotherapy. In certain embodiments, the administered amount or dosage of the agent, the additional agent (e.g., second or third agent), or all, is lower (e.g., at least 20%, at least 30%, at least 40%, or at least 50%) than the amount or dosage of each agent used individually. In other embodiments, the amount or dosage of agent, the additional agent (e.g., second or third agent), or all, that results in a desired effect (e.g., treatment of a fibrotic disease or disorder) is lower (e.g., at least 20%, at least 30%, at least 40%, or at least 50% lower) than the amount or dosage of each agent individually required to achieve the same therapeutic effect.

In some embodiments, the cotherapy is a drug, such as aspirin, acetaminophen, non-steroidal anti-inflammatory drugs, steroids, nerve blockers, and analgesic drugs common in the art.

In some embodiments, the cotherapy is a drug for muscular dystrophies, including but not limited to deflazacourt, eteplirsen, casimersen, golodirsen, ataluren, givinostat, viltolarsen, pamrevlumab, SRP-9001, SRP-5051, DS-5141B, SCAAV9.U7.ACCA, PF-06939926, SGT-001, or AT702.

In some embodiments, the cotherapy is a drug for spinal muscular atrophy, including but not limited to Spinraza, Zolgensma, Evrysdi, SRK-015, CK-2127107, LMI070, AVXS-101, BIIB110, or p38aMAPK inhibitors.

In some embodiments, the cotherapy is a drug for cerebral palsy, stroke, traumatic brain injury, or peripheral nerve injury, including but not limited to anticholinergics such as Benztropine mesylate, Carbidopa-levodopa (Sinemet), Glycopyrrolate (Robinul), Procyclidine hydrochloride (Kemadrin), and Trihexyphenidyl hydrochloride; anticonvulsants such as Gabapentin (Neurontin), Lamotrigine (Lamictal), Oxcarbazepine (Trileptal), Topiramate (Topamax), and Zonisamide (Zonegran); or antispastics i.e. muscle relaxants such as Baclofen, Botulinum toxin, Diazepam (Valium®), Dantrolene, Flexeril (Cyclobenzadrine), Dantrium (Dantrolene), or Tizanidine.

In some embodiments, the cotherapy is physical therapy.

In some embodiments, the cotherapy is a surgical intervention, including but not limited to surgical release, capsular release, or surgical repair.

In some embodiments, the cotherapy is an energy-based technique, including but not limited to radiofrequency energy application e.g. radiofrequency ablation, thermal energy application or removal e.g. cryoablation, sonic energy application e.g. ultrasound-based therapeutic techniques, electrical energy application e.g. transcutaneous electrical nerve stimulation (TENs), or other electromagnetic energy application or removal methods such as light exposure.

In some embodiments, the cotherapy is an exoskeleton designed to assist ambulation or other motion in patients with ambulatory or other motion-based dysfunction.

### Fusion Protein

In another aspect, provided herein is a fusion protein. Generally, the fusion protein comprises a relaxin domain and at least one of a solubility domain or a self-cleaving domain. The solubility domain is linked to the relaxin domain via a protease cleavable linker.

In some embodiments of any one of the aspects described herein, the fusion protein comprises a relaxin domain and a self-cleaving domain. The self-cleaving domain can be linked to the N-terminal of the relaxin domain or the C-terminal of the relaxin domain. In some preferred embodiments, the self-cleaving domain is linked to the C-terminal of the relaxin domain. It is noted that the relaxin domain and the self-cleaving domain can be linked directly to each other, e.g., via a direct bond, or via a linker described herein.

In some embodiments of any one of the aspects described herein, the fusion protein comprises a relaxin domain and a solubility domain. The solubility domain can be linked to the N-terminal of the relaxin domain or the C-terminal of the relaxin domain. In some preferred embodiments, the solubility domain is linked to the N-terminal of the relaxin domain. It is noted that the relaxin domain and the solubility domain can be linked directly to each other, e.g., via a direct bond, or via a linker described herein. In some embodiments, the relaxin domain and the solubility domain are linked to each other via the protease cleavable domain.

In some embodiments of any one of the aspects described herein, the fusion protein comprises a relaxin domain and a protease cleavable domain. The protease cleavable domain can be linked to the N-terminal of the relaxin domain or the C-terminal of the relaxin domain. In some preferred embodiments, the protease cleavable domain is linked to the N-terminal of the relaxin domain. It is noted that the relaxin domain and the protease cleavable domain can be linked directly to each other, e.g., via a direct bond, or via a linker described herein. In some embodiments, the protease cleavable domain connects the relaxin domain and the solubility domain.

In some embodiments of any one of the aspects described herein, the fusion protein further comprises an epitope or affinity tag. For example, the fusion protein comprises two different affinity tags. One of the affinity tags can be at one end of the fusion protein and the other affinity tag can be at an opposite end of the fusion protein. For example, the fusion protein comprises a first affinity tag at N-terminal of the fusion protein and a second affinity tag at C-terminal of the fusion protein. In some embodiments, a first epitope or affinity tag is at a position N-terminal of the solubility domain and a second epitope or affinity tag is at a position C-terminal of the self-cleaving domain.

In some embodiments of any one of the aspects, the fusion protein comprises, in an N to C direction, a first affinity tag, a solubility domain, a protease cleavable domain, a self-cleaving domain, and a second affinity tag. In some embodiments of any one of the aspects, the fusion protein comprises, in an N to C direction, a first affinity tag, a self-cleaving domain, a relaxin domain, a protease cleavable domain, a solubility domain, and a second affinity tag.

In some embodiments of any one of the aspects described herein, the fusion protein comprises the A chain and B chain of the relaxin domain linked together by a linker described herein.

It is noted that the A chain and B chain can be linked in any desired orientation. For example, C-terminal of the A chain can be linked to the N-terminal of the B chain. Alternatively, the C-terminal of the B chain can be linked to the N-terminal of the A chain. The A chain and B chain can be linked by a linker, e.g., a linker described herein. In some embodiments, the C-terminal of the B chain and the N-terminal of the A chain are linked together by a polypeptide linker. In some preferred embodiments, the C-terminal of the B chain and the N-terminal of the A chain are linked together by a linker comprising the amino acid sequence selected from SEQ ID NOS: 44-50.

### Polynucleotide Encoding the Fusion Protein

The invention also provides a polynucleotide encoding a fusion protein described herein. The skilled person will understand that, due to the degeneracy of the genetic code, a given polypeptide can be encoded by different polynucleotides. These “variants” are encompassed herein.

In some embodiments, a polynucleotide encoding a fusion protein described herein is comprised in a vector. In some embodiments, a nucleic acid sequence encoding a fusion protein described herein is operably linked to a vector. The term “vector”, as used herein, refers to a nucleic acid construct designed for delivery to a host cell or for transfer between different host cells. As used herein, a vector can be viral or non-viral. The term “vector” encompasses any genetic element that is capable of replication when associated with the proper control elements and that can transfer gene sequences to cells. A vector can include, but is not limited to, a cloning vector, an expression vector, a plasmid, phage, transposon, cosmid, chromosome, virus, virion, etc.

In some embodiments, the vector is recombinant, e.g., it comprises sequences originating from at least two different sources. In some embodiments of any of the aspects, the vector comprises sequences originating from at least two different species. In some embodiments of any of the aspects, the vector comprises sequences originating from at least two different genes, e.g., it comprises a fusion protein or a nucleic acid encoding an expression product which is operably linked to at least one non-native (e.g., heterologous) genetic control element (e.g., a promoter, suppressor, activator, enhancer, response element, or the like).

In some embodiments, the vector or polynucleotide described herein is codon-optimized, e.g., the native or wild-type sequence of the nucleic acid sequence has been altered or engineered to include alternative codons such that altered or engineered nucleic acid encodes the same polypeptide expression product as the native/wild-type sequence, but will be transcribed and/or translated at an improved efficiency in a desired expression system. In some embodiments, the expression system is an organism other than the source of the native/wild-type sequence (or a cell obtained from such organism). In some embodiments, the vector and/or nucleic acid sequence described herein is codon-optimized for expression in a mammal or mammalian cell, e.g., a mouse, a murine cell, or a human cell. In some embodiments, the vector and/or nucleic acid sequence described herein is codon-optimized for expression in a human cell. In some embodiments, the vector and/or nucleic acid sequence described herein is codon-optimized for expression in a yeast or yeast cell. In some embodiments, the vector and/or nucleic acid sequence described herein is codon-optimized for expression in a bacterial cell. In some embodiments, the vector and/or nucleic acid sequence described herein is codon-optimized for expression in an E. coli cell.

As used herein, the term “expression vector” refers to a vector that directs expression of an RNA or polypeptide from sequences linked to transcriptional regulatory sequences on the vector. The sequences expressed will often, but not necessarily, be heterologous to the cell. An expression vector may comprise additional elements, for example, the expression vector may have two replication systems, thus allowing it to be maintained in two organisms, for example in human cells for expression and in a prokaryotic host for cloning and amplification.

As used herein, the term “viral vector” refers to a nucleic acid vector construct that includes at least one element of viral origin and has the capacity to be packaged into a viral vector particle. The viral vector can contain the nucleic acid encoding an antibody or antigen-binding fragment thereof as described herein in place of non-essential viral genes. The vector and/or particle may be utilized for the purpose of transferring any nucleic acids into cells either in vitro or in vivo. Numerous forms of viral vectors are known in the art.

As used herein, the term “expression host” includes any living cell containing the genetic material for the recombinant expression of the relaxin or an analog, a fragment or a variant thereof

### Kits

A fusion protein, polynucleotide described herein can be provided in a kit, e.g., as a component of a kit. For example, the kit includes (a) a fusion protein or polynucleotide described herein, and, optionally (b) informational material. The informational material can be descriptive, instructional, marketing or other material that relates to the methods described herein and/or the use of a fusion protein or polynucleotide described herein for the methods described herein. The informational material of the kits is not limited in its form. In some embodiments, the informational material can include information about production of the fusion protein or the polynucleotide encoding the fusion protein, their molecular weight, concentration, date of expiration, batch or production site information, and so forth.

The informational material of the kits is not limited in its form. In many cases, the informational material, e.g., instructions, is provided in print but can also be in other formats, such as computer readable material.

Components of the kit, e.g., the fusion protein and/or the polynucleotide can be provided in any form, e.g., liquid, dried or lyophilized form. When the fusion protein or the polynucleotide is provided in a liquid solution, the liquid solution preferably is an aqueous solution. When the fusion protein or the polynucleotide is provided as a dried form, reconstitution generally is by the addition of a suitable solvent. The solvent, e.g., sterile water or buffer, can optionally be provided in the kit.

The kit can include one or more containers for the components of the kit. In some embodiments, the kit contains separate containers, dividers or compartments for the different components of the kit.

In some embodiments, the kit can further comprise additional components and/or reagents for practicing the methods described herein using the fusion protein and/or the polynucleotide described herein.

Embodiments of the various aspects described herein can be described by the following numbered embodiments:

Embodiment 1: A method of producing a soluble recombinant relaxin or variants or analogues thereof, the method comprising: recombinantly expressing a fusion protein in a host cell, wherein the fusion protein comprises: (i) a first affinity tag; (ii) a solubility domain; (iii) a protease cleavable domain; (iv) a relaxin domain; (v) a self-cleaving domain; and (vi) a second affinity tag, wherein the first and second affinity tags are different; (b) releasing the fusion protein from the host cell; (c) cleaving the protease cleavable domain of the isolated fusion protein to release the solubility domain from the fusion protein; (d) cleaving the self-cleaving domain of the cleaved fusion protein to release the relaxin domain; (e) optionally, cleaving the released relaxin domain to produce a cleaved relaxin domain; and (f) optionally, incubating the cleaved relxain domain under oxidation-reduction conditions to produce soluble relaxin.

Embodiment 2: The method of Embodiment 1, wherein said releasing the fusion protein from the host cell comprises lysing the host cell.

Embodiment 3: The method of Embodiment 2 or 3, further comprising a step of isolating the released fusion protein prior to cleaving the protease cleavable domain.

Embodiment 4: The method of Embodiment 3, wherein said isolating the released fusion protein comprises affinity chromatography using the first affinity tag.

Embodiment 5: The method of Embodiment 4, wherein said affinity chromatography using the first affinity tag comprises metal ion affinity chromatograph (IMAC).

Embodiment 6: The method of any one of Embodiments 1-5, wherein the first affinity tag comprises a His-tag.

Embodiment 7: The method of any one of Embodiments 1-6, wherein the first affinity comprises the amino acid sequence of SEQ ID NO: 51

Embodiment 7: The method of any one of Embodiments 1-7, wherein the protease cleavable domain comprises a cleavage site of protease selected from the group consisting of potyvirus Ma proteases, potyvirus HC proteases, potyvirus P1 (P35) proteases, byovirus Ma proteases, byovirus RNA-2-encoded proteases, aphthovirus L proteases, enterovirus 2A proteases, rhinovirus 2A proteases, picorna 3C proteases, comovirus 24K proteases, nepovirus 24K proteases, rice tungro spherical virus (RTSV) 3C-like protease, parsnip yellow fleck virus (PYVF) 3C-like protease, heparin, thrombin, factor Xa, PreScission protease, MMP, and enterokinase, or an analogue or a variant thereof.

Embodiment 8: The method of any one of Embodiments 1-8, wherein protease cleavable domain comprises a cleavage site of TEV, or an analogue or a variant thereof.

Embodiment 9: The method of any one of Embodiments 1-9, wherein protease cleavable domain comprises an amino acid sequence selected from the group consisting of SEQ ID NOS: 1-25 and 44-50, or an analogue or a variant thereof.

Embodiment 10: The method of any one of Embodiments 1-10, wherein said cleaving the self-cleaving domain is in presence of an affinity matrix capable of binding with the second affinity tag.

Embodiment 11: The method of any one of Embodiments 1-11, wherein the second affinity tag comprises a chitin binding domain, or an analogue or a variant thereof.

Embodiment 13: The method of any one of Embodiment 1-12, wherein the second affinity tag comprises the amino acid sequence of SEQ ID NO: 56, or an analogue or a variant thereof.

Embodiment 14: The method of any one of Embodiments 1-13, wherein the self-cleaving domain comprises an amino acid sequence of an auto-catalytic domain of an intein.

Embodiment 15: The method of any one of Embodiments 1-14, wherein the self-cleaving domain comprises an amino acid sequence of an auto-catalytic domain of an intein selected from the group consisting of Mxe GyrA intein, Ssp DnaB mini-intein, Mth RIR1 intein and Sce VMA1 intein, or an analogue or a variant thereof.

Embodiment 16: The method of any one of Embodiments 1-15, the self-cleaving domain comprises an amino acid sequence of Mxe GyrA intein (SEQ ID NO: 27), or an analogue or a variant thereof.

Embodiment 17: The method of any one of Embodiments 1-16, wherein the self-cleaving domain cleavage is in presence of a conjugate-ligand.

Embodiment 18: The method of Embodiment 17, wherein the cleavage of the self-cleaving domain results in linking of the conjugate-ligand to the relaxin domain.

Embodiment 19: The method of any one of Embodiments 1-18, wherein the step of cleaving the released relaxin domain comprises incubating the released relaxin domain with a type ii transmembrane serine protease (TTSP)

Embodiment 20: The method of Embodiment 19, wherein the TTSP is an enteropeptidase (enterokinase).

Embodiment 21: The method of any one of Embodiments 1-20, wherein the relaxin domain comprises a relaxin-like (RLN) peptide, an insulin-like (INSL), an analogue or a variant thereof.

Embodiment 22: The method of any one of Embodiments 1-21, wherein the relaxin domain comprises relaxin-1 (RLN1), relaxin-2 (RLN2), relaxin-3 (RLN3), INSL3, INSL4, INSL5 or INSL6, or an analogue or a variant thereof.

Embodiment 23: The method of any one of Embodiments 1-22, wherein the relaxin domain comprises an amino acid sequence selected from the group consisting of SEQ ID NOS: 28-43, or an analogue or a variant thereof.

Embodiment 24: The method of any one of Embodiments 1-22, wherein the relaxin domain comprises human relaxin-2 (hRLX-2) or an analogue or a variant thereof.

Embodiment 25: The method of any one of Embodiments 1-24, wherein the fusion protein further comprises a conjugate-ligand for targeting of the relaxin domain.

Embodiment 26: A recombinant relaxin produced by a method of any of Embodiments 1-25.

Embodiment 27: The relaxin of Embodiment 26, wherein the relaxin is formulated for intravenous, intramuscular, subcutaneous, intradermal, intranasal, oral, transcutaneous, mucosal or intraarticular administration to a subject.

Embodiment 28: The relaxin of Embodiment 27 or 28, wherein the relaxin is formulated as a gel, a cream, an ointment, a lotion, a drop, a suppository, a spray, a liquid or a powder composition.

Embodiment 29: The relaxin of any one of Embodiments 26-28, wherein the relaxin is formulated in a sustained release composition.

Embodiment 30: A method comprising administering to a subject a relaxin of Embodiment 26.

Embodiment 31: The method of Embodiment 30, wherein the subject is need of treatment for a fibrotic disease.

Embodiment 32: The method of Embodiment 31, wherein the fibrotic disease is selected from the group consisting of stiffened fibrotic joint capsules, lung fibrosis, liver fibrosis, kidney fibrosis, heart disease, intestinal disease, skin conditions, urogenital and gynecological conditions and ocular diseases.

Embodiment 33: The method of Embodiment 31 or 32, wherein the fibrotic disease is selected from the group consisting of idiopathic pulmonary fibrosis, cystic fibrosis, hypertension, hepatitis B or C, non-alcoholic steatohepatitis, non-alcoholic fatty liver disease, Cholestasis, autoimmune hepatitis cirrhosis, chronic kidney disease, end-stage renal disease, renal interstitial fibrosis, heart failure, myocardial infarction, aortic stenosis, hypertrophic cardiomyopathy, Crohn's disease, inflammatory bowel disease, enteropathies, other intestinal fibrosis, scleroderma, keloids, hypertrophic scars, cellulite, Peyronie's disease, uterine fibroids, Congenital Fibrosis of the Extraocular Muscles, subretinal fibrosis, epiretinal fibrosis, and corneal fibrosis.

Embodiment 34: The method of any one of Embodiments 31-33, wherein the fibrotic disease is arthrofibrosis or a stiffened fibrotic joint.

Embodiment 35: A fusion protein comprising: (a) a first affinity tag; (b) a solubility domain; (c) a protease cleavable domain; (d) a relaxin domain; (e) a self-cleaving domain; and (f) a second affinity domain.

Embodiment 36: The fusion protein of Embodiment 35, wherein the solubility domain and the self-cleaving domain are linked to the opposite ends of the relaxin domain.

Embodiment 37: Embodiment 37: The fusion protein of Embodiment 35 or 36, wherein the solubility domain is linked to the N-terminal of the relaxin domain.

Embodiment 38: The fusion protein of any one of Embodiments 35-37, wherein the solubility domain is linked to the relaxin domain via the protease cleavable domain.

Embodiment 39: The fusion protein of any one of Embodiments 35-38, wherein the solubility domain is linked to the C-terminal of the relaxin domain.

Embodiment 40: The fusion protein of any one of Embodiments 35-39, wherein the first tag is linked to the solubility domain.

Embodiment 41: The fusion protein of any one of Embodiments 35-40, wherein the second tag is lined to the self-cleaving domain.

Embodiment 42: A composition comprising a fusion protein of any one of Embodiments 35-41.

Embodiment 43: A kit comprising a fusion protein of any one of Embodiments 35-41

Embodiment 44: A cell comprising a fusion protein of any one of Embodiments.

Embodiment 45: A polynucleotide encoding a fusion protein of any one of Embodiments 35-41.

Embodiment 46: A composition comprising a polynucleotide of Embodiment 45.

Embodiment 47: A kit comprising a polynucleotide of Embodiment 45.

### Some Selected Definitions

Unless otherwise defined herein, scientific and technical terms used in connection with the present application shall have the meanings that are commonly understood by those of ordinary skill in the art to which this invention belongs. It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims. Definitions of common terms in immunology and molecular biology can be found in The Merck Manual of Diagnosis and Therapy, 20th Edition, published by Merck Sharp & Dohme Corp., 2018 (ISBN 0911910190, 978-0911910421); Robert S. Porter et al. (eds.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), W. W. Norton & Company, 2016 (ISBN 0815345054, 978-0815345053); Lewin's Genes XI, published by Jones & Bartlett Publishers, 2014 (ISBN-1449659055); Michael Richard Green and Joseph Sambrook, Molecular Cloning: A Laboratory Manual, 4th ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN 047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs. If there is an apparent discrepancy between the usage of a term in the art and its definition provided herein, the definition provided within the specification shall prevail.

The terms “decrease”, “reduced”, “reduction”, or “inhibit” are all used herein to mean a decrease by a statistically significant amount. In some embodiments, “reduce,” “reduction” or “decrease” or “inhibit” typically means a decrease by at least 10% as compared to a reference level (e.g. the absence of a given treatment or agent) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level. A decrease can be preferably down to a level accepted as within the range of normal for an individual without a given disorder.

The terms “increased”, “increase”, “enhance”, or “activate” are all used herein to mean an increase by a statically significant amount. In some embodiments, the terms “increased”, “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. In the context of a marker or symptom, a “increase” is a statistically significant increase in such level.

As used herein, a “subject” means a human or animal. Usually the animal is a vertebrate such as a primate, non-primate, rodent, birds, domestic animal or game animal. Primates include humans and non-human primates such as chimpanzees, cynomolgus monkeys, spider monkeys, and macaques, e.g., Rhesus. Rodents include mice, rats, woodchucks, guinea pigs, ferrets, rabbits and hamsters. Domestic and game animals include cows, horses, llamas, camels, pigs, goats, sheeps, deer, bison, buffalo, feline species, e.g., domestic cat, canine species, e.g., dog, fox, wolf, avian species, e.g., chicken, emu, ostrich, and fish, e.g., trout, catfish, whale and salmon. Birds include, ducks and geese. In some embodiments, the subject is a mammal, e.g., a primate, e.g., a human. The terms, “individual,” “patient” and “subject” are used interchangeably herein.

Preferably, the subject is a mammal. The mammal can be a human, non-human primate, mouse, rat, dog, cat, horse, or cow, but is not limited to these examples. Mammals other than humans can be advantageously used as subjects that represent animal models of viral infection. A subject can be male or female.

A subject can be one who has been previously diagnosed with or identified as suffering from or having a disease or disorder in need of treatment (e.g., a disease or disorder associated with fibrosis) or one or more complications related to such a disease or disorder, and optionally, have already undergone treatment for the disease or disorder or the one or more complications related to the disease or disorder. Alternatively, a subject can also be one who has not been previously diagnosed as having such disease or disorder (e.g., a disease or disorder associated with fibrosis) or related complications. For example, a subject can be one who exhibits one or more risk factors for the disease or disorder or one or more complications related to the disease or disorder or a subject who does not exhibit risk factors.

In an embodiment, the subject is a human, such as a human being assessed for a stiffened joint, a human at risk for developing a stiffened joint, a human having a stiffened joint, and/or a human being treated for a stiffened joint.

As used herein, the terms “treat,” “treatment,” “treating,” or “amelioration” refer to therapeutic treatments, wherein the object is to reverse, alleviate, ameliorate, inhibit, slow down or stop the progression or severity of a condition, such as a condition associated with fibrosis, e.g. a fibrotic disease and disorder. The term “treating” includes reducing or alleviating at least one adverse effect or symptom of a condition, such as a condition associate with fibrotic disease and disorder (e.g., inflammation, stiffening of a joint, contracture of a joint, contracture of a joint not caused by muscle contracture, contracture of a joint associated with muscle contracture, pain, loss of mobility, difficulty breathing, muscle stiffness, muscle dysfunction, skin dimpling, keloid scarring, burn-associated scarring). Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation of, or at least slowing of, progress or worsening of symptoms compared to what would be expected in the absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, remission (whether partial or total), and/or decreased mortality, whether detectable or undetectable. The term “treatment” of a disease also includes providing relief from the symptoms or side-effects of the disease (including palliative treatment).

As used herein, an “effective amount,” is intended to include the amount of the agent e.g., relaxin or an analog, a fragment or a variant thereof, that, when administered to a subject via a depot having a stiffened joint, is sufficient to affect treatment of the stiffened joint (e.g., by diminishing, ameliorating or maintaining the stiffened joint or one or more symptoms of the stiffened joint). The “effective amount” may vary depending on the sequence of the agent, how the agent is administered, the severity of the joint stiffness and the history, age, weight, family history, genetic makeup, the types of preceding or concomitant treatments, if any, and other individual characteristics of the subject to be treated.

The term “effective amount,” as used herein, is also intended to include the amount of agent e.g., relaxin or an analog, a fragment or a variant thereof, that, when administered to a subject in a depot with a stiffened joint, and either currently or not yet experiencing or displaying symptoms of the stiffened joint, such as pain on movement or restriction of the movement or range of movement of the joint affected by the joint stiffness, and/or a subject at risk of developing a stiffened joint, is sufficient to prevent or ameliorate the stiffened joint or one or more of its symptoms. Ameliorating the stiffened joint includes slowing the course of the progression of the joint stiffness or reducing the severity of later-developing joint stiffness.

As used herein, the terms “treating”, “treat” or “treatment”, when used in reference to a stiffened joint, refers a beneficial or desired result including, but not limited to, alleviation or amelioration of one or more symptoms associated with a stiffened joint (e.g., pain on movement of the joint, loss of motion of the joint or loss of the range of motion of the joint); diminishing the restriction of movement resulting from a stiffened joint; stabilization (i.e., not worsening) of the joint stiffness; amelioration or palliation of the restriction of movement resulting from a stiffened joint (e.g., pain on movement of the joint, loss of motion of the joint or loss of the range of motion of the joint) whether detectable or undetectable.

As used herein, “prevention” or “preventing,” when used in reference to a stiffened joint, refers to a reduction in the likelihood that a subject, e.g., a human subject, will develop a symptom associated with such a stiffened joint, or a reduction in the frequency and/or duration of a symptom associated with a stiffened joint. The likelihood of developing a stiffened joint is reduced, for example, when a subject having one or more risk factors for a stiffened joint either fails to develop a stiffened joint or develops a stiffened joint with less severity relative to a population having the same risk factors and not receiving treatment as described herein. The failure to develop a stiffened joint, or the reduction in the development of a symptom associated with stiffened joint (e.g., by at least about 10%), or the exhibition of delayed symptoms (e.g., delayed by days, weeks, months or years) is considered effective prevention.

As used herein, the term “administering,” refers to the placement of a therapeutic (e.g., a relaxin or a variant or analogue thereof described herein) or composition as disclosed herein into a subject by a method or route which results in at least partial delivery of the therapeutic to the subject. Compositions, e.g., pharmaceutical composition comprising agents as disclosed herein can be administered by any appropriate route which results in an effective treatment in the subject.

As used herein, the terms “protein” and “polypeptide” are used interchangeably to designate a series of amino acid residues, connected to each other by peptide bonds between the alpha-amino and carboxy groups of adjacent residues. The terms “protein”, and “polypeptide” refer to a polymer of amino acids, including modified amino acids (e.g., phosphorylated, glycated, glycosylated, etc.) and amino acid analogs, regardless of its size or function. “Protein” and “polypeptide” are often used in reference to relatively large polypeptides, whereas the term “peptide” is often used in reference to small polypeptides, but usage of these terms in the art overlaps. The terms “protein” and “polypeptide” are used interchangeably herein when referring to a gene product and fragments thereof. Thus, exemplary polypeptides or proteins include gene products, naturally occurring proteins, homologs, orthologs, paralogs, fragments and other equivalents, variants, fragments, and analogs of the foregoing.

As used herein, the term “conjugate-ligand” refers to, without limitation, a single-domain camelid antibody fragment, a peptide sequence, an amino acid, a polynucleotide, a synthetic polymer, a small molecule, or a combination of any of the previous. In some embodiments, the conjugate-ligand possesses an amino-terminal cysteine amino acid or other thiol-functionalized group. In a preferred embodiment, the conjugate-ligand is L-cysteine. In some specific embodiments, the conjugate-ligand is a cysteine-PEG co-polymer. In some specific embodiments the conjugate-ligand is a PEG polymer functionalized to allow for interaction with the intein domain. In some specific embodiments, the conjugate-ligand is a peptide encoding a targeting domain for extracellular matrix proteins. In some embodiments the conjugate-ligand is comprised of a carbohydrate (e.g. hyaluronic acid, dextran, chitosan, alginate). In some embodiments, the conjugate-ligand is comprised of a glycoprotein (e.g. collagen, chondroitin sulfate). In some embodiments, the conjugate-ligand is comprised of a lipid or glycolipid.

The terms “wild-type” or “wt” or “native” as used herein is meant an amino acid sequence or a nucleotide sequence that is found in nature, including allelic variations. A wild-type protein, polypeptide, antibody, immunoglobulin, IgG, polynucleotide, DNA, RNA, and the like has an amino acid sequence or a nucleotide sequence that has not been intentionally modified.

In the various embodiments described herein, it is further contemplated that variants (naturally occurring or otherwise), alleles, homologs, conservatively modified variants, and/or conservative substitution variants of any of the particular polypeptides described are encompassed. As to amino acid sequences, one of skill will recognize that individual substitutions, deletions or additions to a nucleic acid, peptide, polypeptide, or protein sequence which alters a single amino acid or a small percentage of amino acids in the encoded sequence is a “conservatively modified variant” where the alteration results in the substitution of an amino acid with a chemically similar amino acid and retains the desired activity of the polypeptide. Such conservatively modified variants are in addition to and do not exclude polymorphic variants, interspecies homologs, and alleles consistent with the invention.

The term “amino acid substitution” refers to the replacement of at least one existing amino acid residue in a predetermined or native amino acid sequence with a different “replacement” amino acid. A given amino acid can be replaced by a residue having similar physiochemical characteristics, e.g., substituting one aliphatic residue for another (such as Ile, Val, Leu, or Ala for one another), or substitution of one polar residue for another (such as between Lys and Arg; Glu and Asp; or Gln and Asn). Other such conservative substitutions, e.g., substitutions of entire regions having similar hydrophobicity characteristics, are well known. Polypeptides comprising conservative amino acid substitutions can be tested confirm that a desired activity and specificity of a native or reference polypeptide is retained.

Amino acids can be grouped according to similarities in the properties of their side chains (in A. L. Lehninger, in Biochemistry, second ed., pp. 73-75, Worth Publishers, New York (1975)): (1) non-polar: Ala (A), Val (V), Leu (L), Ile (I), Pro (P), Phe (F), Trp (W), Met (M); (2) uncharged polar: Gly (G), Ser (S), Thr (T), Cys (C), Tyr (Y), Asn (N), Gln (Q); (3) acidic: Asp (D), Glu (E); (4) basic: Lys (K), Arg (R), His (H). Alternatively, naturally occurring residues can be divided into groups based on common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; (6) aromatic: Trp, Tyr, Phe. Non-conservative substitutions will entail exchanging a member of one of these classes for another class. Particular conservative substitutions include, for example; Ala into Gly or into Ser; Arg into Lys; Asn into Gln or into His; Asp into Glu; Cys into Ser; Gln into Asn; Glu into Asp; Gly into Ala or into Pro; His into Asn or into Gln; Ile into Leu or into Val; Leu into Ile or into Val; Lys into Arg, into Gln or into Glu; Met into Leu, into Tyr or into Ile; Phe into Met, into Leu or into Tyr; Ser into Thr; Thr into Ser; Trp into Tyr; Tyr into Trp; and/or Phe into Val, into Ile or into Leu.

The term “amino acid insertion” refers to the insertion of one or more additional amino acids into a predetermined or native amino acid sequence. The insertion can be one, two, three, four, five, or up to twenty amino acid residues.

The term “amino acid deletion” refers to removal of at least one amino acid from a predetermined or native amino acid sequence. The deletion can be one, two, three, four, five, or up to twenty amino acid residues.

In some embodiments, the polypeptide described herein (or a nucleic acid encoding such a polypeptide) can be a functional fragment of one of the amino acid sequences described herein. As used herein, a “functional fragment” is a fragment or segment of a polypeptide which retains at least 50% of the wild-type reference polypeptide's activity according to the assays described herein. A functional fragment can comprise conservative substitutions of the sequences disclosed herein.

In some embodiments, the polypeptide described herein can be a variant of a sequence described herein. In some embodiments, the variant is a conservatively modified variant. Conservative substitution variants can be obtained by mutations of native nucleotide sequences, for example. A “variant,” as referred to herein, is a polypeptide substantially homologous to a native or reference polypeptide, but which has an amino acid sequence different from that of the native or reference polypeptide because of one or a plurality of deletions, insertions or substitutions. Variant polypeptide-encoding DNA sequences encompass sequences that comprise one or more additions, deletions, or substitutions of nucleotides when compared to a native or reference DNA sequence, but that encode a variant protein or fragment thereof that retains activity. A wide variety of PCR-based site-specific mutagenesis approaches are known in the art and can be applied by the ordinarily skilled artisan to generate and test artificial variants.

The term “nucleic acid” refers to a deoxyribonucleotide or ribonucleotide and polymers thereof in either single strand or double strand form. The term “nucleic acid” is used interchangeably with gene, nucleotide, polynucleotide, cDNA, DNA, and mRNA. The polynucleotides can be in the form of RNA or DNA. Polynucleotides in the form of DNA, cDNA, genomic DNA, nucleic acid analogs, and synthetic DNA are within the scope of the present invention. Unless specifically limited the term encompasses nucleic acids containing known analogues of natural nucleotides that have similar binding propertied as the natural nucleic acid. Unless specifically limited, a particular nucleotide sequence also encompasses conservatively modified variants thereof (for example, those containing degenerate codon substitutions) and complementary sequences as well as the as well as the sequences specifically described.

The polynucleotides can be composed of any polyribonucleotide or polydeoxyribonucleotide, which can be unmodified RNA or DNA or modified RNA or DNA. For example, polynucleotides can be composed of single or double stranded regions, mixed single or double stranded regions. In addition, the polynucleotides can be triple stranded regions containing RNA or DNA or both RNA and DNA. Modified polynucleotides include modified bases, such as tritylated bases or unusual bases such as inosine. A variety of modification can be made to RNA and DNA, thus polynucleotide includes chemically, enzymatically, or metabolically modified forms.

The DNA may be double-stranded or single-stranded, and if single stranded, may be the coding (sense) strand or non-coding (anti-sense) strand. The coding sequence that encodes the polypeptide may be identical to the coding sequence provided herein or may be a different coding sequence, which sequence, as a result of the redundancy or degeneracy of the genetic code, encodes the same polypeptides as the DNA provided herein.

A variant DNA or amino acid sequence can be at least 90%, at least 91%, at least 92%, at least 93%, at least 94%, at least 95%, at least 96%, at least 97%, at least 98%, at least 99%, or more, identical to a native or reference sequence. The degree of homology (percent identity) between a native and a mutant sequence can be determined, for example, by comparing the two sequences using freely available computer programs commonly employed for this purpose on the world wide web (e.g. BLASTp or BLASTn with default settings).

In some embodiments of the various aspects described herein, a polypeptide, nucleic acid, or cell as described herein can be engineered. As used herein, “engineered” refers to the aspect of having been manipulated by the hand of man. For example, a polynucleotide is considered to be “engineered” when at least one aspect of the polynucleotide, e.g., its sequence, has been manipulated by the hand of man to differ from the aspect as it exists in nature.

The term “statistically significant” or “significantly” refers to statistical significance and generally means a two standard deviations (2SD) or greater difference.

Other than in the operating examples, or where otherwise indicated, all numbers expressing quantities of ingredients or reaction conditions used herein should be understood as modified in all instances by the term “about.” The term “about” when used in connection with percentages can mean±1%. In some embodiments of the various aspects described herein, the term “about” when used in connection with percentages can mean±5%.

As used herein, the term “comprising” means that other elements can also be present in addition to the defined elements presented. The use of “comprising” indicates inclusion rather than limitation.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of additional elements that do not materially affect the basic and novel or functional characteristic(s) of that embodiment of the invention.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this invention, suitable methods and materials are described below. The abbreviation, “e.g.” is derived from the Latin exempli gratia, and is used herein to indicate a non-limiting example. Thus, the abbreviation “e.g.” is synonymous with the term “for example.”

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

As used herein, a “reference level” refers to a normal, otherwise unaffected cell population or tissue (e.g., a biological sample obtained from a healthy subject, or a biological sample obtained from the subject at a prior time point, e.g., a biological sample obtained from a patient prior to being diagnosed with a fibrotic disease or disorder, or a biological sample that has not been contacted with an agent disclosed herein).

As used herein, an “appropriate control” refers to an untreated, otherwise identical cell or population (e.g., a patient who was not administered an agent described herein, or was administered by only a subset of agents described herein, as compared to a non-control cell).

The description of embodiments of the invention is not intended to be exhaustive or to limit the invention to the precise form disclosed. While specific embodiments of, and examples for, the invention are described herein for illustrative purposes, various equivalent modifications are possible within the scope of the invention, as those skilled in the relevant art will recognize. For example, while method steps or functions are presented in a given order, alternative embodiments may perform functions in a different order, or functions may be performed substantially concurrently. The teachings of the invention provided herein can be applied to other procedures or methods as appropriate. The various embodiments described herein can be combined to provide further embodiments. Aspects of the invention can be modified, if necessary, to employ the compositions, functions and concepts of the above references and application to provide yet further embodiments of the invention. Moreover, due to biological functional equivalency considerations, some changes can be made in protein structure without affecting the biological or chemical action in kind or amount. These and other changes can be made to the invention in light of the detailed description. All such modifications are intended to be included within the scope of the appended claims.

Specific elements of any of the foregoing embodiments can be combined or substituted for elements in other embodiments. Furthermore, while advantages associated with certain embodiments of the invention have been described in the context of these embodiments, other embodiments may also exhibit such advantages, and not all embodiments need necessarily exhibit such advantages to fall within the scope of the invention.

The technology described herein is further illustrated by the following examples which in no way should be construed as being further limiting.

### EXAMPLES

### Example 1. An Exemplary Purification Schema for the Production of Human Relaxin-2

In this example, the recombinant relaxin was expressed as a fusion protein in E. coli. The E. coil containing fusion protein construct was streaked onto a Luria broth (miller's formulation) with 1% agarose and 50 mg/ml kanamycin and grown at 37° C. overnight. A singled colony was inoculated into LB media containing 50 mg/ml kanamycin and grown at 37° C. and shaking at 200 RPM for 16 hours. That saturated culture was subcultured 1:100 into LB media containing 50 mg/ml kanamycin and grown at 37° C. and shaking at 200 RPM for ˜2 hours. Exact timing was determined by sampling the optical density (600 nm) of the culture until OD600=0.5 was achieved. The culture was then induced with 1 mM IPTG for 4 hours at 37° C. and shaking at 200 RPM.

Induced cells were harvest via centrifugation at 5000×g for 15 minutes at 4° C. The wet cell pellet was frozen at −80° C. The frozen pellet was thawed on ice in lysis buffer (50 mM MES, 150 mM NaCl, 2 mM MgCl2, 1 mM PMSF, 100 mg/ml hen egg white lysozyme, 1:10000 Pierce universal nuclease, pH 6). Cell suspensions was brought to room temperature for 15 minutes to allow for lysozyme activity. Cells were lysed by two passages through a microfluidizer at 15 k PSI. Cell lysate was fractioned via centrifugation at 45,000×g for 45 min at 4° C. The insoluble fraction of the cell lysate was discarded.

The soluble fraction of the cell lysate was incubated with cobalt bound 6% cross-linked agarose resin for 45 min at 4° C. mixing at 5 RPM. Prior to incubation the resin was washed with 20× bed volume of H₂O and then equilibrated with 20× bed volume of cobalt resin wash buffer (50 mM MOPS, 500 mM NaCl, 5 mM Imidazole, 0.1% Triton X-100, pH 7). Equal volume of wash buffer was added to the soluble fraction and then incubation was performed as previously described. After incubation, the resin was washed with 30× bed volume of cobalt resin wash buffer. 5× bed volume of cobalt resin elution buffer (50 mM MOPS, 500 mM NaCl, 300 mM Imidazole, pH 7) was added to the resin, the resin was resuspended to a slurry, and then fractioned.

Pooled fractions were dialyzed against Tobacco Etch Virus protease buffer (50 mM MOPS, 150 mM NaCl, 5 mM TCEP, pH 7) with a 1:25 dilution of 1 mg/ml TEV protease added to the pooled fractions. Dialysis was performed with pre-wetted 5 k MWCO dialysis tubbing, for 72 hours, with a 1:1000 exchange volume ratio and buffer changed at 4 hours, 24 hours, and 48 hours.

Digested construct was incubated with chitin resin for 30 mins at 4° C. mixing at 5 RPM. Prior to incubation, the resin was washed with 20× bed volume of H₂O and then equilibrated with 20× bed volume of chitin resin wash buffer (20 mM Na-HEPES, 500 mM NaCl, 0.1% Triton X-100, pH 8.5). Equal volume of wash buffer was added to the digested construct and then incubation was performed as previously described. After incubation, the resin was washed with 30× bed volume of chitin resin wash buffer. Resin was incubated with 3× bed volumes of intein cleavage buffer (20 mM Na-HEPES, 500 mM NaCl, 30 mM MESNA, 5 mM TCEP, 10 mM L-Cysteine, pH 8.5) for 48 hours at 4° C. without mixing. Collected flow through contains the linearized relaxin.

Linearized relaxin was dialyzed against enterokinase cleavage buffer (20 mM Na-HEPES, 100 mM NaCl, 2 mM CaCl2, pH 8) for 24 hours. Buffer was changed after 4 hours and 12 hours and a 1:1000 exchange ratio was used. Dialyzed linearized relaxin was incubated with 1 unit enterokinase (light chain)/2 mg relaxin for 16 hours at room temperature at 5 RPM. After trypsin protease cleanup, the digested relaxin product was incubated with 10 mM oxidized glutathione and 2 mM reduced glutathione for 24 hours at 4° C. mixing at 5 RPM.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their invention prior to the filing date of the present application. Nothing in this regard should be construed as an admission that the inventors are not entitled to antedate such invention by virtue of prior invention or for any other reason. All statements as to the date or representation as to the contents of these documents is based on the information available to the applicants and does not constitute any admission as to the correctness of the dates or contents of these documents.

### LISTING OF SEQUENCES

SEQ ID NO: 1
QXXYXES
SEQ ID NO: 2
QXXYFXG
SEQ ID NO: 3
DDDDK
SEQ ID NO: 4
RGYZ
SEQ ID NO: 5
EXXYXQ-(G/S)
SEQ ID NO: 6
GTVRFQ-(G/S)
SEQ ID NO: 7
RXR/K-R
SEQ ID NO: 8
S/TXA-S/AG
SEQ ID NO: 9
LEVLFQ-GP
SEQ ID NO: 10
DDDDK-X
SEQ ID NO: 11
(D/E)R-M
SEQ ID NO: 12
LVPR-GS
SEQ ID NO: 13
PLGLAG
SEQ ID NO: 14
IE/DGR-X
SEQ ID NO: 15
PGAAH-Y
SEQ ID NO: 16
MYKR-EAD
SEQ ID NO: 17
IEPD-X
SEQ ID NO: 18
DEVD-X
SEQ ID NO: 19
GPLGMLSQ
SEQ ID NO: 20
GPLGLWAQ
SEQ ID NO: 21
GPLGLAG
SEQ ID NO: 22
KKNPAELIGPVD
SEQ ID NO: 23
KKQPAANLVAPED
SEQ ID NO: 24
ENLYFQG
SEQ ID NO: 25
ENLYFQS
(maltose binding protein [escherichia coli])
SEQ ID NO: 26
MKIEEGKLVIWINGDKGYNGLAEVGKKFEKDTGIKVTVEHPDKLEEKFPQVAATGDGPDIIFWAHDR
FGGYAQSGLLAEITPDKAFQDKLYPFTWDAVRYNGKLIAYPIAVEALSLIYNKDLLPNPPKTWEEIP
ALDKELKAKGKSALMFNLQEPYFTWPLIAADGGYAFKYENGKYDIKDVGVDNAGAKAGLTFLVDLIK
NKHMNADTDYSIAEAAFNKGETAMTINGPWAWSNIDTSKVNYGVTVLPTFKGQPSKPFVGVLSAGIN
AASPNKELAKEFLENYLLTDEGLEAVNKDKPLGAVALKSYEEELVKDPRIAATMENAQKGEIMPNIP
QMSAFWYAVRTAVINAASGRQTVDEALKDAQTNSSSNNNNN
(DNA gyrase A subunit [mycobacterium xenopi)
SEQ ID NO: 27
CITGDALVALPEGESVRIADIVPGARPNSDNAIDLKVLDRHGNPVLADRLFHSGEHPVYTVRTVEGL
RVTGTANHPLLCLVDVAGVPTLLWKLIDEIKPGDYAVIQRSAFSVDCAGFARGKPEFAPTTYTVGVP
GLVRFLEAHHRDPDAQAIADELTDGRFYYAKVASVTDAGVQPVYSLRVDTADHAFITNGFVSHAT
>gill13543609IgbIAAH05956.1I Relaxin 1 [Homo sapiens]
SEQ ID NO: 28
MPRLFLFHLLEFCLLLNQFSRAVAAKWKDDVIKLCGRELVRAQIAICGMSTWSKRSLSQEDAP
QTPRPVAEIVPSFINKDIETIIIMLEFIANLPPELKAALSERQPSLPELQQYVPALKDSNLSFEE
FKKLIRNRQSEAADSNPSELKYLGLDTHSQKKRRPYVALFEKCCLIGCTKRSLAKYC
>gill19579171IgblEAW58767.1I relaxin 1, isoform CRA_a [Homo
sapiens]
SEQ ID NO: 29
MPRLFLFHLLEFCLLLNQFSRAVAAKWKDDVIKLCGRELVRAQIAICGMSTWSKRSLSQEDAPQTPR
PEIVPSFINKDIETIIIMLEFIANLPPELKAALSERQPSLPELQQYVPALKDSNLSFEEFKKLIRN
RQSEAADSNPSELKYLGLDTHSQKKRRPYVALFEKCCLIGCTKRSLAKYC
>gi119579172gbEAW58768.1 relaxin 1, isoform CRA b [Homo sapiens]
SEQ ID NO: 30
MPRLFLFHLLEFCLLLNQFSRAVAANWKDDVIKLCGRELVRAQIAICGMSTWSKRSLSQEDAPQTPRPVA
GISSSLLRRRLFEDHDGPSFLV
>gill19579173Hg-bEAW58769.1 relaxin 1, isoform CRA c [Homo
sapiens]
SEQ ID NO: 31
MLEFIANLPPELKAALSERQPSLPELQQYVPALKDSNLSFEEFKKLIRNRQSEAADSNPSELKYL
GLDTHSQKKRRPYVALFEKCCLIGCTKRSLAKYC
>gi116497221HgbAA126416.1 Relaxin 2 [Homo sapiens]
SEQ ID NO: 32
MPRLFFFHLLGVCLLLNQFSRAVADSWMEEVIKLCGRELVRAQIAICGMSTWSKRSLSQEDAPQTPRPVAE
IVPSFINKDTETINMMSEFVANLPQELKLTLSEMQPALPQLQQHVPVLKDSSLLFEEFKKLIRNRQSEAAD
SSPSELKYLGLDTHSRKKRQLYSAIANKCCHVGCTKRSLARFC
>gill16496899IgbIAAI26420.1I Relaxin 2 [Homo sapiens]
SEQ ID NO: 33
MPRLFFFHLLGVCLLLNQFSRAVADSWMEEVIKLCGRELVRAQIAICGMSTWSKRSLSQEDAPQT
PRPVAEIVPSFINKDIETINMMSEFVANLPQELKLILSEMQPALPQLQQHVPVLKDSSLLFEEFK
KLIRNRQSEAADSSPSELKYLGLDTHSRKKRQLYSALANKCCHVGCTKRSLARFC
>gil313884020IgbIADR83496.1I relaxin 2, partial [synthetic
construct]
SEQ ID NO: 34
MPRLFFFHLLGVCLLLNQFSRAVADSWMEEVIKLCGRELVRAQIAICGMSTWSKRSLSQEDAPQTP
RPVAEIVPSFINKDIETINMMSEFVANLPQELKLILSEMQPALPQLQQHVPVLKDSSLLFEEFKKL
IRNRQSEAADSSPSELKYLGLDTHSRKKRQLYSALANKCCHVGCTKRSLARFC
>gi119604794gbEAW84388.1 relaxin 3 [Homo sapiens]
SEQ ID NO: 35
MARYMLLLLLAVWVLTGELWPGAEARAAPYGVRLCGREFIRAVIFTCGGSRWRRSDILAHEAMGDT
FPDADADEDSLAGELDEAMGSSEWLALTKSPQAFYRGRPSWQGTPGVLRGSRDVLAGLSSSCCKWG
CSKSEISSLC
>gi187954661gbAAI40936.1 Relaxin 3 [Homo sapiens]
SEQ ID NO: 36
MARYMLLLLLAVWVLTGELWPGAEARAAPYGVRLCGREFIRAVIFTCGGSRWRRSDILAHEAMGDT
FPDADADEDSLAGELDEAMGSSEWLALTKSPQAFYRGRPSWQGTPVVLRGSRDVLAGLSSSCCKWG
CSKSEISSLC
>gi17484096HgbAAL40345.1AF447451 1 relaxin 3 [Homo sapiens]
SEQ ID NO: 37
MARYMLLLLLAVWVLTGELWPGAEARAAPYGVRLCGREFIRAVIFTCGGSRWRRSDILAHEAMGD
TFPDADADEDSLAGELDEAMGSSEWLALTKSPQAFYRGRPSWQGTPGVLRGSRDVLAGLSSSCCK
WGCSKSEISSLC
>gi317373369spJ [symbol] 51460.2INSL3 HUMAN RecName: Full = Insulin-like
3;
SEQ ID NO: 38
MDPRLPAWALVLLGPALVFALGPAPTPEMREKLCGHHEVRALVRVCGGPRWSTEARRPATGGDR
ELLQWLERRHLLHGLVADSNLTLGPGLQPLPQTSHHHRHHRAAATNPARYCCLSGCTQQDLLTLC
PY
>gi119579176gbEAW58772.1 insulin-like 4 (placenta) [Homo
sapiens]
SEQ ID NO: 39
MASLFRSYLPAIWLLLSQLLRESLAAELRGCGPRFGKHLLSYCPMPEKTFTTTPGGWLLESGRPKEM
VSTSNNKDGQALGTTSEFIPNLSPELKKPLSEGQPSLKKIILSRKKRSGRHRFDPFCCEVICDDGTS
VKLCT
>gi20070773HgbAAH26254.1 Insulin-like 4 (placenta) [Homo
sapiens]
SEQ ID NO: 40
MASLFRSYLPAIWLLLSQLLRESLAAELRGCGPRFGKHLLSYCPMPEKTFTTTPGGWLLESGRPKEM
VSTSNNKDGQALGTTSEFIPNLSPELKKPLSEGQPSLKKIILSRKKRSGRHRFDPFCCEVICDDGTS
VKLCT
>gi37183171AQ89389.1 INSL5 [Homo sapiens]
SEQ ID NO: 41
MKGSIFTLFLFSVLFAISEVRSKESVRLCGLEYIRTVIYICASSRWRRHLEGIPQAQQAETGNSF
QLPHKREFSEENPAQNLPKVDASGEDRLWGGQMPTEELWKSKKHSVMSRQDLQTLCCTDGCSMTD
LSALC
>giH4768935gbAAD29686.1AF133816 1 insulin-like peptide INSL5
[Homo sapiens]
SEQ ID NO: 42
MKGSIFTLFLFSVLFAISEVRSKESVRLCGLEYIRTVIYICASSRWRRHLEGIPQAQQAETGNSFQL
PHKREFSEENPAQNLPKVDASGEDRLWGGQMPTEELWKSKKHSVMSRQDLQTLCCIDGCSMTDLSAL
C
>gik5059419gbAAD39003.1AF156094 1 insulin-like protein 6 [Homo
sapiens]
SEQ ID NO: 43
MPRLLRLSLLWLGLLLVRFSRELSDISSARKLCGRYLVKEIEKLCGHANWSQFRFEEETPFSRLIAQ
ASEKVEAYSPYQFESPQTASPARGRGTNPVSTSWEEAVNSWEMQSLPEYKDKKGYSPLGKTREFSSS
HNINVYIHENAFFQKKRRNKIKTLSNLFWGHHPQRKRRGYSEKCCLTGCTKEELSIACLPYIDFKRL
KEKRSSLVTKIY
>Flexible chain linker variant 1 [synthetic construct]
SEQ ID NO: 44
GGGGSGGGG
>Flexible chain linker variant 1 [synthetic construct]
SEQ ID NO: 45
GGGGSGGGGSG
>Flexible chain linker variant 1 [synthetic construct]
SEQ ID NO: 46
GGGSGGG
>Flexible chain linker variant 1 [synthetic construct]
SEQ ID NO: 47
GGSGGSGGSG
>Flexible chain linker variant 1 [synthetic construct]
SEQ ID NO: 48
GGSGGSGG
>Flexible chain linker variant 1 [synthetic construct]
SEQ ID NO: 49
GGGGSGGGGSGGGGS
>Flexible chain linker variant 1 [synthetic construct]
SEQ ID NO: 50
GSGGGSGGGGSGGGSG
SEQ ID NO: 51
HHHHHH
SEQ ID NO: 52
YPYDVPDYA
SEQ ID NO: 53
EQKLISEEDL
SEQ ID NO: 54
DTYRYI
SEQ ID NO: 55
DYKDDDDK
>chitin binding subunit [synthetic construct]
SEQ ID NO: 56
GLTGLNSGLTTNPGVSAWQVNTAYTAGQLVTYNGKTYKCLQPHTSLAGWEPSNVPALWQLQ
SEQ ID NO: 57
(GlyₓSer)ₙ, where x is 2, 3, 4, 5 or 6, and n is 1, 2, 3, 4, 5, 6,
7, 8, 9 or 10

What is claimed is:

1. A method of producing a soluble recombinant relaxin or variants or analogues thereof, the method comprising:
a. recombinantly expressing a fusion protein in a host cell, wherein the fusion protein comprises:
i. a first affinity tag;
ii. a solubility domain;
iii. a protease cleavable domain;
iv. a relaxin domain;
v. a self-cleaving domain; and
vi. a second affinity tag, wherein the first and second affinity tags are different;
b. releasing the fusion protein from the host cell;
c. cleaving the protease cleavable domain of the isolated fusion protein to release the solubility domain from the fusion protein;
d. cleaving the self-cleaving domain of the cleaved fusion protein to release the relaxin domain;
e. cleaving the released relaxin domain to produce a cleaved relaxin domain;
f. incubating the cleaved relxain domain under oxidation-reduction conditions to produce soluble relaxin.

2. The method of claim 1, wherein said releasing the fusion protein from the host cell comprises lysing the host cell.

3. The method of claim 2, further comprising a step of isolating the released fusion protein prior to cleaving the protease cleavable domain.

4. The method of claim 3, wherein said isolating the released fusion protein comprises affinity chromatography using the first affinity tag.

5. The method of claim 4, wherein said affinity chromatography using the first affinity tag comprises metal ion affinity chromatograph (IMAC).

6. The method of claim 1, wherein the protease cleavable domain comprises a cleavage site of protease selected from the group consisting of potyvirus Ma proteases, potyvirus HC proteases, potyvirus P1 (P35) proteases, byovirus Ma proteases, byovirus RNA-2-encoded proteases, aphthovirus L proteases, enterovirus 2A proteases, rhinovirus 2A proteases, picorna 3C proteases, comovirus 24K proteases, nepovirus 24K proteases, rice tungro spherical virus (RTSV) 3C-like protease, parsnip yellow fleck virus (PYVF) 3C-like protease, heparin, thrombin, factor Xa, PreScission protease, MMP, and enterokinase.

7. The method of claim 6, wherein protease cleavable domain comprises a cleavage site of TEV.

8. The method of claim 1, wherein said cleaving the self-cleaving domain is in presence of an affinity matrix capable of binding with the second affinity tag.

9. The method of claim 1, wherein the self-cleaving domain comprises an amino acid sequence of an auto-catalytic domain an intein.

10. The method of claim 1, wherein the self-cleaving domain cleavage is in presence of a conjugate-ligand.

11. The method of claim 10, wherein the cleavage of the self-cleaving domain results in linking of the conjugate-ligand to the relaxin domain.

12. The method of claim 11, wherein the intein is selected from the group consisting of Mxe GyrA intein, Ssp DnaB mini-intein, Mth RIR1 intein and Sce VMA1 intein.

13. The method of claim 1, wherein the step of cleaving the released relaxin domain comprises incubating the released relaxin domain with a type II transmembrane serine protease (TTSP)

14. The method of claim 13, wherein the TTSP is an enteropeptidase (enterokinase).

15. The method of claim 1, wherein the relaxin domain comprises a relaxin-like (RLN) peptide, an insulin-like (INSL), an analogue or a variant thereof.

16. The method of claim 15, wherein the relaxin domain comprises relaxin-1 (RLN1), relaxin-2 (RLN2), relaxin-3 (RLN3), INSL3, INSL4, INSL5 or INSL6, or an analogue or a variant thereof, or wherein the relaxin domain comprises human relaxin-2 (hRLX-2) or an analogue or a variant thereof.

17. The method of claim 1, wherein the fusion protein further comprises a conjugate-ligand for targeting of the relaxin domain.

18. A recombinant relaxin produced by a method of claim 1.

19. The relaxin of claim 18, wherein the relaxin is formulated for intravenous, intramuscular, subcutaneous, intradermal, intranasal, oral, transcutaneous, mucosal or intraarticular administration to a subject.

20. The relaxin of claim 18, wherein the relaxin is formulated as a gel, a cream, an ointment, a lotion, a drop, a suppository, a spray, a liquid or a powder composition.

21. The relaxin of claim 18, wherein the relaxin is formulated in a sustained release composition.

22. A method comprising administering to a subject a relaxin of claim 21.

23. The method of claim 22, wherein the subject is need of treatment for a fibrotic disease.

24. The method of claim 23, wherein the fibrotic disease is selected from the group consisting of stiffened fibrotic joint capsules, lung fibrosis, liver fibrosis, kidney fibrosis, heart disease, intestinal disease, skin conditions, urogenital and gynecological conditions and ocular diseases; or
wherein the fibrotic disease is selected from the group consisting of idiopathic pulmonary fibrosis, cystic fibrosis, hypertension, hepatitis B or C, non-alcoholic steatohepatitis, non-alcoholic fatty liver disease, Cholestasis, autoimmune hepatitis cirrhosis, chronic kidney disease, end-stage renal disease, renal interstitial fibrosis, heart failure, myocardial infarction, aortic stenosis, hypertrophic cardiomyopathy, Crohn's disease, inflammatory bowel disease, enteropathies, other intestinal fibrosis, scleroderma, keloids, hypertrophic scars, cellulite, Peyronie's disease, uterine fibroids, Congenital Fibrosis of the Extraocular Muscles, subretinal fibrosis, epiretinal fibrosis, and corneal fibrosis; or
wherein the fibrotic disease is arthrofibrosis or a stiffened fibrotic joint.

25. A fusion protein comprising:
a. a first affinity tag;
b. a solubility domain;
c. a protease cleavable domain;
d. a relaxin domain;
e. a self-cleaving domain; and
f. a second affinity domain.


SEQUENCE LISTING (59 sequences)

SEQ ID NO: 1 - protein, 7 aa, Unknown
  Description of Unknown protease cleavable site sequence
  MOD_RES (2)..(3): Any amino acid
  MOD_RES (5)..(5): Any amino acid
QXXYXES

SEQ ID NO: 2 - protein, 7 aa, Unknown
  Description of Unknown protease cleavable site sequence
  MOD_RES (2)..(3): Any amino acid
  MOD_RES (6)..(6): Any amino acid
QXXYFXG

SEQ ID NO: 3 - protein, 5 aa, Unknown
  Description of Unknown protease cleavable site sequence
DDDDK

SEQ ID NO: 4 - protein, 4 aa, Unknown
  Description of Unknown protease cleavable site sequence
  MOD_RES (3)..(3): Any negatively charged amino acid
  MOD_RES (4)..(4): Any cationic or neutral amino acid
RGXX

SEQ ID NO: 5 - protein, 7 aa, Tobacco etch virus
  MOD_RES (2)..(3): Any amino acid
  MOD_RES (5)..(5): Any amino acid
  MOD_RES (7)..(7): G or S
EXXYXQX

SEQ ID NO: 6 - protein, 7 aa, Tobacco vein mottling virus
  MOD_RES (7)..(7): G or S
GTVRFQX

SEQ ID NO: 7 - protein, 4 aa, Unknown
  Description of Unknown Furin sequence
  MOD_RES (2)..(2): Any amino acid
  MOD_RES (3)..(3): R or K
RXXR

SEQ ID NO: 8 - protein, 5 aa, Infectious pancreatic necrosis virus
  MOD_RES (1)..(1): S or T
  MOD_RES (2)..(2): Any amino acid
  MOD_RES (4)..(4): S or A
XXAXG

SEQ ID NO: 9 - protein, 8 aa, Enterovirus sp.
LEVLFQGP

SEQ ID NO: 10 - protein, 6 aa, Unknown
  Description of Unknown Enterokinase sequence
  MOD_RES (6)..(6): Any amino acid
DDDDKX

SEQ ID NO: 11 - protein, 3 aa, Unknown
  Description of Unknown Enterokinase sequence
  MOD_RES (1)..(1): D or E
XRM

SEQ ID NO: 12 - protein, 6 aa, Unknown
  Description of Unknown Thrombin sequence
LVPRGS

SEQ ID NO: 13 - protein, 6 aa, Unknown
  Description of Unknown MMP sequence
PLGLAG

SEQ ID NO: 14 - protein, 5 aa, Unknown
  Description of Unknown Factor Xa protease sequence
  MOD_RES (2)..(2): E or D
  MOD_RES (5)..(5): Any amino acid
IXGRX

SEQ ID NO: 15 - protein, 6 aa, Unknown
  Description of Unknown Genenase I sequence
PGAAHY

SEQ ID NO: 16 - protein, 7 aa, Unknown
  Description of Unknown KEX2 protease sequence
MYKREAD

SEQ ID NO: 17 - protein, 5 aa, Unknown
  Description of Unknown Granzyme B sequence
  MOD_RES (5)..(5): Any amino acid
IEPDX

SEQ ID NO: 18 - protein, 5 aa, Unknown
  Description of Unknown Caspase-3 sequence
  MOD_RES (5)..(5): Any amino acid
DEVDX

SEQ ID NO: 19 - protein, 8 aa, Unknown
  Description of Unknown protease cleavable domain sequence
GPLGMLSQ

SEQ ID NO: 20 - protein, 8 aa, Unknown
  Description of Unknown protease cleavable domain sequence
GPLGLWAQ

SEQ ID NO: 21 - protein, 7 aa, Unknown
  Description of Unknown protease cleavable domain sequence
GPLGLAG

SEQ ID NO: 22 - protein, 12 aa, Unknown
  Description of Unknown protease cleavable domain sequence
KKNPAELIGPVD

SEQ ID NO: 23 - protein, 13 aa, Unknown
  Description of Unknown protease cleavable domain sequence
KKQPAANLVAPED

SEQ ID NO: 24 - protein, 7 aa, Unknown
  Description of Unknown protease cleavable domain sequence
ENLYFQG

SEQ ID NO: 25 - protein, 7 aa, Unknown
  Description of Unknown protease cleavable domain sequence
ENLYFQS

SEQ ID NO: 26 - protein, 376 aa, Escherichia coli
MKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELVKDPRIAATMENAQKGEIMPNIPQMSAFWYAVRTAVINAASGRQTVDEALKDAQTNSSSNNNNN

SEQ ID NO: 27 - protein, 199 aa, Mycobacterium xenopi
CITGDALVALPEGESVRIADIVPGARPNSDNAIDLKVLDRHGNPVLADRLFHSGEHPVYTVRTVEGLRVTGTANHPLLCLVDVAGVPTLLWKLIDEIKPGDYAVIQRSAFSVDCAGFARGKPEFAPTTYTVGVPGLVRFLEAHHRDPDAQAIADELTDGRFYYAKVASVTDAGVQPVYSLRVDTADHAFITNGFVSHAT

SEQ ID NO: 28 - protein, 185 aa, Homo sapiens
MPRLFLFHLLEFCLLLNQFSRAVAAKWKDDVIKLCGRELVRAQIAICGMSTWSKRSLSQEDAPQTPRPVAEIVPSFINKDIETIIIMLEFIANLPPELKAALSERQPSLPELQQYVPALKDSNLSFEEFKKLIRNRQSEAADSNPSELKYLGLDTHSQKKRRPYVALFEKCCLIGCTKRSLAKYC

SEQ ID NO: 29 - protein, 183 aa, Homo sapiens
MPRLFLFHLLEFCLLLNQFSRAVAAKWKDDVIKLCGRELVRAQIAICGMSTWSKRSLSQEDAPQTPRPEIVPSFINKDIETIIIMLEFIANLPPELKAALSERQPSLPELQQYVPALKDSNLSFEEFKKLIRNRQSEAADSNPSELKYLGLDTHSQKKRRPYVALFEKCCLIGCTKRSLAKYC

SEQ ID NO: 30 - protein, 92 aa, Homo sapiens
MPRLFLFHLLEFCLLLNQFSRAVAAKWKDDVIKLCGRELVRAQIAICGMSTWSKRSLSQEDAPQTPRPVAGISSSLLRRRLFEDHDGPSFLV

SEQ ID NO: 31 - protein, 99 aa, Homo sapiens
MLEFIANLPPELKAALSERQPSLPELQQYVPALKDSNLSFEEFKKLIRNRQSEAADSNPSELKYLGLDTHSQKKRRPYVALFEKCCLIGCTKRSLAKYC

SEQ ID NO: 32 - protein, 185 aa, Homo sapiens
MPRLFFFHLLGVCLLLNQFSRAVADSWMEEVIKLCGRELVRAQIAICGMSTWSKRSLSQEDAPQTPRPVAEIVPSFINKDTETINMMSEFVANLPQELKLTLSEMQPALPQLQQHVPVLKDSSLLFEEFKKLIRNRQSEAADSSPSELKYLGLDTHSRKKRQLYSALANKCCHVGCTKRSLARFC

SEQ ID NO: 33 - protein, 185 aa, Homo sapiens
MPRLFFFHLLGVCLLLNQFSRAVADSWMEEVIKLCGRELVRAQIAICGMSTWSKRSLSQEDAPQTPRPVAEIVPSFINKDIETINMMSEFVANLPQELKLILSEMQPALPQLQQHVPVLKDSSLLFEEFKKLIRNRQSEAADSSPSELKYLGLDTHSRKKRQLYSALANKCCHVGCTKRSLARFC

SEQ ID NO: 34 - protein, 185 aa, Artificial Sequence; identical to SEQ ID NO: 33
  Description of Artificial Sequence Synthetic polypeptide

SEQ ID NO: 35 - protein, 142 aa, Homo sapiens
MARYMLLLLLAVWVLTGELWPGAEARAAPYGVRLCGREFIRAVIFTCGGSRWRRSDILAHEAMGDTFPDADADEDSLAGELDEAMGSSEWLALTKSPQAFYRGRPSWQGTPGVLRGSRDVLAGLSSSCCKWGCSKSEISSLC

SEQ ID NO: 36 - protein, 142 aa, Homo sapiens [sequence omitted]

SEQ ID NO: 37 - protein, 142 aa, Homo sapiens; identical to SEQ ID NO: 35

SEQ ID NO: 38 - protein, 131 aa, Homo sapiens
MDPRLPAWALVLLGPALVFALGPAPTPEMREKLCGHHEVRALVRVCGGPRWSTEARRPATGGDRELLQWLERRHLLHGLVADSNLTLGPGLQPLPQTSHHHRHHRAAATNPARYCCLSGCTQQDLLTLCPY

SEQ ID NO: 39 - protein, 139 aa, Homo sapiens
MASLFRSYLPAIWLLLSQLLRESLAAELRGCGPRFGKHLLSYCPMPEKTFTTTPGGWLLESGRPKEMVSTSNNKDGQALGTTSEFIPNLSPELKKPLSEGQPSLKKIILSRKKRSGRHRFDPFCCEVICDDGTSVKLCT

SEQ ID NO: 40 - protein, 139 aa, Homo sapiens; identical to SEQ ID NO: 39

SEQ ID NO: 41 - protein, 135 aa, Homo sapiens
MKGSIFTLFLFSVLFAISEVRSKESVRLCGLEYIRTVIYICASSRWRRHLEGIPQAQQAETGNSFQLPHKREFSEENPAQNLPKVDASGEDRLWGGQMPTEELWKSKKHSVMSRQDLQTLCCTDGCSMTDLSALC

SEQ ID NO: 42 - protein, 135 aa, Homo sapiens; identical to SEQ ID NO: 41

SEQ ID NO: 43 - protein, 213 aa, Homo sapiens
MPRLLRLSLLWLGLLLVRFSRELSDISSARKLCGRYLVKEIEKLCGHANWSQFRFEEETPFSRLIAQASEKVEAYSPYQFESPQTASPARGRGTNPVSTSWEEAVNSWEMQSLPEYKDKKGYSPLGKTREFSSSHNINVYIHENAFFQKKRRNKIKTLSNLFWGHHPQRKRRGYSEKCCLTGCTKEELSIACLPYIDFKRLKEKRSSLVTKIY

SEQ ID NO: 44 - protein, 9 aa, Artificial Sequence
  Description of Artificial Sequence Synthetic peptide
GGGGSGGGG

SEQ ID NO: 45 - protein, 11 aa, Artificial Sequence
  Description of Artificial Sequence Synthetic peptide
GGGGSGGGGSG

SEQ ID NO: 46 - protein, 7 aa, Artificial Sequence
  Description of Artificial Sequence Synthetic peptide
GGGSGGG

SEQ ID NO: 47 - protein, 10 aa, Artificial Sequence
  Description of Artificial Sequence Synthetic peptide
GGSGGSGGSG

SEQ ID NO: 48 - protein, 8 aa, Artificial Sequence
  Description of Artificial Sequence Synthetic peptide
GGSGGSGG

SEQ ID NO: 49 - protein, 15 aa, Artificial Sequence
  Description of Artificial Sequence Synthetic peptide
GGGGSGGGGSGGGGS

SEQ ID NO: 50 - protein, 16 aa, Artificial Sequence
  Description of Artificial Sequence Synthetic peptide
GSGGGSGGGGSGGGSG

SEQ ID NO: 51 - protein, 6 aa, Artificial Sequence
  Description of Artificial Sequence Synthetic 6xHis tag
HHHHHH

SEQ ID NO: 52 - protein, 9 aa, Artificial Sequence
  Description of Artificial Sequence Synthetic peptide
YPYDVPDYA

SEQ ID NO: 53 - protein, 10 aa, Artificial Sequence
  Description of Artificial Sequence Synthetic peptide
EQKLISEEDL

SEQ ID NO: 54 - protein, 6 aa, Artificial Sequence
  Description of Artificial Sequence Synthetic peptide
DTYRYI

SEQ ID NO: 55 - protein, 8 aa, Artificial Sequence
  Description of Artificial Sequence Synthetic peptide
DYKDDDDK

SEQ ID NO: 56 - protein, 61 aa, Artificial Sequence
  Description of Artificial Sequence Synthetic polypeptide
GLTGLNSGLTTNPGVSAWQVNTAYTAGQLVTYNGKTYKCLQPHTSLAGWEPSNVPALWQLQ

SEQ ID NO: 57 - protein, 70 aa, Artificial Sequence
  Description of Artificial Sequence Synthetic polypeptide
  SITE (1)..(70): This sequence may encompass 1-10 “(Gly)x-Ser” repeating units where x=2-6
  See specification as filed for detailed description of substitutions and preferred embodiments
GGGGGGSGGGGGGSGGGGGGSGGGGGGSGGGGGGSGGGGGGSGGGGGGSGGGGGGSGGGGGGSGGGGGGS

SEQ ID NO: 58 - protein, 7 aa, Unknown
  Description of Unknown protease cleavable site sequence
  MOD_RES (2)..(3): Any amino acid
  MOD_RES (5)..(5): Any amino acid
EXXYXQS

SEQ ID NO: 59 - protein, 7 aa, Unknown
  Description of Unknown protease cleavable site sequence
  MOD_RES (2)..(3): Any amino acid
  MOD_RES (6)..(6): Any amino acid
EXXYFXG